SEQUENCE LISTING (2 sequences)

SEQ ID NO: 1 - DNA, 10976 bp, Homo sapiens
gagctcggatcccatcgcagctaccgcgatgagaggcgctcgcggcgcctgggattttctctgcgttctgctcctactgcttcgcgtccagacaggtgggacaccgcggctggctccccgaccgtgcgactactcgcgaagcctgtgccctgggagggtggtaccgccatggcatccggagagaggactgcgggccctcagtgggcctgcgttccagcctccggggagactccaggtggccctcggactctccggcgccctgcctcgctcacctgcgcgaggagaccccagctgctggtggtgggggacgcgaatccggggttcttcgggaatggggacagcaagagggggttaggcgtgagcgaggctgcaggctccgtgcgagtttggggtggcttttgtgccgacgttgcgcgggggcggaggcgggggctcagggtttgcaccgagcgccttctctctcggtgcgaggccggccgcagcttccttttgttaaaagttgcgtgtgtgtgacggcgcccgggctgcagcctcaacctcctgggcttaagtgatctcccacctcagcctcccgcctcagcctcccatgtagctgggaaaacaggttcccactaccatgcccagctaatgttttttctagtttttgtagatgtgttgggcggtgggggcgtctcactgcgttgcccaggctggtctcgaactcctaggctcaagcaatcttcccacctcagcctcccagagtgctgggattacaggcgtgacgacggcacctggccagcagtttgttctttaaaccctgaaatgtatgtgaggaccatgtgtcacactagcatggacgttttcccagcatctagcatggtgctttgtagatgataattaatgaataggtatttgaatacatggaggcatgcatggctgaatgaagtggctgttgtaaaatttctagggttcaggtttcatattcagagcctaaagtttgcatcttataaactaaatagtttcctatctaggaaacctatttaggcattagggtgttaaaacaggtgtatcattttctgccttagtgtttagagatttgtgaatagttctccttttgatgaacattgccatgtaaagagagttatacagaaataactgaattcacacagttttaaggaaaagctattctatgtcatggtcatgtatattctgccaaagagagtgacaggccagtagtttcttttttctttttccccatagtgtgagattttgtttctgttgtttctaagctgggtgtctgcatgtccacactgcgaagatggcccatatcagaatgaaaacttgacccaattgtatgtttagcccagagaaggctggggcattcagcacatcctggcttggggaccaaatgtgaccctcaggattaattgagggttggagaaaataattttatagatgaacttaagacattttagacagaactctcttttcagccataaatagcagggcagctttgtcctatttttattgtagagtacacagaagatggaactcagtattggaagaagtgctttatttcgccaaggaagaagatcatactcaacacgattctgtttttcttggcaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagaggtaaatgcttggctttctgcagcaggtcatgtcactttaggagggttgcttttatgacaccgcagtttcatctatgaaatggcaataatgatagtactgatcatgggaggggcaatttgaagattaaatgagattaagtgtaatggtccaagcttagtgcgtgatacatggaaagcgtttaataaatgttaattctcaatagtactagatggataaattttgcttttgtttacacagaaaaaagcagccatttgggccactagtcatgaaaggcaacatattagatctttaaaagtgtttcagtgtctgtgaccagccattccaactactgatttggatatgcttcttatagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccaggtaccttgctttcttatctgcctctggagttgagaactcacttatctaaagagacttctcttctcgttgatcgtaagctgtacacatttgaggagaaatggtaaatcaaaatttcatgctataatacaaattatttgaggggccacatttcttttcattctagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcaggtgagtgaatcgcttcattcttctcatgttctgtctctgtgggagatgataagttttctctttcagaagagtctgtcctgaaactgcctcgactagtgcgtctgtcagaggagaagttaattgctgctatttttaatttatctaggaaagattctgaatataaattatatggtaatcttcatttttttttctccttttctgaaaccagcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtaggtaaatacctctatgggaatgtttaaattactggcagtagtgaaagaagaaattattagacagtttcttttttatgtaaatggaatgttgaacagattcttagaattttgttatcactgaatgaatgaaaattatccttgtagcctcttgcaatgaaagcacaattctgttttttttgtccagtagttgtagataatgtttctttctgtcttatttcattctaattagataaaggattcattaatatcttccccatgataaacactacagtatttgtaaacgatggagaaaatgtagatttgattgttgaatatgaagcattccccaaacctgaacaccagcagtggatctctctgaacagaaccttcactgataaatgggaagattatcccaagtctgagaatgaaagtaatatcaggtaagaaatggaccttgccctggggattacacattacccccttttccagtgggcttatcagatcttatttctgtaacccgtaaatccacgagaagatacctggtaaagaagaaagtctattttgctaatactttactgaattaaatgagttatatttttcctcaaacaggcatagatttccaggtagaaactgaaaaagacatgccttccaaggcatgctatccacaggtgattgactagttgtcttttctttgtagatacgtaagtgaacttcatctaacgagattaaaaggcaccgaaggaggcacttacacattcctagtgtccaattctgacgtcaatgctgccatagcatttaatgtttatgtgaatagtaagtaacatgaagggctcttttaattttttattcttttaagttgtggctcgtgtttgtaacagctgcaaggactcaacttgctgtactaaaggttgtagggatttagagagggagtgaagtgaatgttgctgaggttttccagcactctgacatatggccatttctgttttcctgtagcaaaaccagaaatcctgacttacgacaggctcgtgaatggcatgctccaatgtgtggcaccaggattcccagagcccacaatagattggtatttttgtccaggaactgagcagaggtgagatgattatttttggcactgcttataatgcagaggggaaggactgcaattcacttgaatttcaaatatgttttctgattttttttaaaaaagctttaactttgttttaaaagtatgccacatcccaagtgttttatgtatttatttattttcctagagtaagccagggcttttgttttcttccctttagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaaggtatatttctttttaatccaatttaaggggatgtttaggctctgtctaccatatcagtcatgattttgagctcaattaaccctcactaaagggagtcgactcgatcccatcctgccaaagtttgtgattccacatttctcttccattgtagagcaaatccatccccacaccctgttcactcctttgctgattggtttcgtaatcgtagctggcatgatgtgcattattgtgatgattctgacctacaaatatttacaggtaaccatttatttgttctctctccagagtgctctaatgactgagacaataattattaaaaggtgatctatttttccctttctccccacagaaacccatgatgaagtacagtggaaggttgttgaggagataaatggaaacaattatgtttacatagacccaacacaacttccttatgatcacaaatgggagtttcccagaaacaggctgagttttggtcagtatgaaacaggggctttccatgtcacctttttgggtacacataacagtgactttaaggaactccagtggcttcctttgttttgttccacctgaaacaatgagttttctgtgaaattgcgccccttttgataggtttgccatagagaacatcgtaggaaaatgtctctggacaacattgtttttaattcctttattgattttgaaactgcacaaatggtccttcaattccaccaccagcaccatcaccacttaccttgttgtcttccttcctacagggaaaaccctgggtgctggagctttcgggaaggttgttgaggcaactgcttatggcttaattaagtcagatgcggccatgactgtcgctgtaaagatgctcaagcgtaagttcctgtatggtactgcatgcgcttgacatcagtttgccagttgtgctttttgctaaaatgcatgtttccaattttagcgagtgcccatttgacagaacgggaagccctcatgtctgaactcaaagtcctgagttaccttggtaatcacatgaatattgtgaatctacttggagcctgcaccattggaggtaaagccgtgtccaagctgccttttattgtctgtcaggttatcaaaacatgacattttaatatgattttggcaatgctagattataaactgcttggaagatttttttacccagactgttgttctctcttgctagattttgttttcctcattgttcttaagaatatatgggattgtattgggactaagtagtctgatccactgaagctgaatattaatggccatgaccacccttgggtatttttatgggaggcagaattaatctatatatctcaccttctttctaaccttttcttatgtgcttttagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctggtaagactgatttacataaatagttagctgttgacaggcagttcatggggaactctttattcaaactttacatgactttcctcaaattggtccagtctattatgtagcaaaggggatgaggaggtagagcatgacccatgagtgcccttctacatgtcccacttgattcagtcatgacttgtttcatctctcccagcagcgatagtactaatgagtacatggacatgaaacctggagtttcttatgttgtcccaaccaaggccgacaaaaggagatctgtgagaataggtgagtacctacctatcaagcaaccaagagtaactttacagagagtatgtatatcatgctaatgtggaatataacatcattccagtagcaatgatgcagaccagttctgctttatggtagcagtgccaatggtcaatggcagttaggggcaagttcacattagttcattcattaccagcctttggtatgtcattgccactgtcttttcctttcctgacctttatggttgtaattgctaagaaaaatcctctcttcctcacaggctcatacatagaaagagatgtgactcccgccatcatggaggatgacgagttggccctagacttagaagacttgctgagcttttcttaccaggtggcaaagggcatggctttcctcgcctccaagaatgtaagtgggagtgattctctaaagagttttgtgttttgtttttttgatttttttttttttttttttttttttgagaacagagcattttagagccatagttaaaagcagaatgtcatttaaaacaaaagtattggattttttataatataagcaacactatagtattaaaaagttagttttcactctttacaagttaaaatgaatttaaatggttttcttttctcctccaacctaatagtgtattcacagagacttggcagccagaaatatcctccttactcatggtcggatcacaaagatttgtgattttggtctagccagagacatcaagaatgattctaattatgtggttaaaggaaacgtgagtacccattctctgcttgacagtcctgcaaaggatttttagtttcaactttcgataaaaattgtttccgtgactttcataatgtaaatcctgtctagggatatcacacattttagcagtcaaatgtatttcagaggtgattgggatcatctgagttcatataggtaaaaggtttttgtgagatggtactcaagttatcactccacatttcagcaacagcagcatctataagaatatcttctgttcaattttgttgagcttctgaattaacattattgactctgttgtgcttctattacaggctcgactacctgtgaagtggatggcacctgaaagcattttcaacgtgtatacacgtttgaaagtgacgtctggtcctatcggatttttctttgggagctgttctctttaggtaaaatgatccttgccaaaagacaacttcattagactcagagcatcttgaagtttcattggtgtcctgcttccttgtgattaacactgctttgcaaactgtgtctcaggaagcagcccctatcctggaatgccggtcgattctaagttctacaagatgatcaaggaaggcttccggatgctcagcctgaacacgcacctgctgaaatgtaagagccaaaaaatttttcctttaggtcacgttttcccttttatttttctttttagagacagaaacccagatgttgagggttttcataacacagtttgaaatgtcacttggattctttatgacacactggtcaaatgtcatttctgtagtttattttcataatctcttgtcaccaaaaatacagaaagtttcagtaatatttcatacatgcagtgttttatgttatctatatgtcagtccatatgtccagttgcatagccctggaattattactgaagttgctggatgcccatacatttgaaaacaagctgagggcattgaggagggatagtaaatggcccttgtcttgcaggtatgacataatgaagacttgctgggatgcagatcccctaaaaagaccaacattcaagcaaattgttcagctaattgagaagcagatttcagagagcaccaatcatgtgagtataccctggccaggcatagaatcccccttctcccagttccaggtgtgtcctcctcctcaggctttcagggtgaggactaacctcccaaccccttctctcctaatcttaggttgcaaattgggcttcaggtaggggaagtaaagcaatggaaactagttcttttaagagttccatcagttagttgtgatcttgacactgtaagtatgccttttgttgctatgttcgttgtagggactgctgtattgactatgggcttgttttctccagatttactccaacttagcaaactgcagccccaaccgacagaagcccgtggtagaccattctgtgcggatcaattctgtcggcagcaccgcttcctcctcccagcctctgcttgtgcacgacgatgtctgagcagaatcagtgtttgggtcacccctccaggaatgatctcttcttttggcttccatgatggttattttcttttctttcaacttgcatccaactccaggatagtgggcaccccactgcaatcctgtctttctgagcacactttagtggccgatgatttttgtcatcagccaccatcctattgcaaaggttccaactgtatatattcccaatagcaacgtagcttctaccatgaacagaaaacattctgatttggaaaaagagagggaggtatggactgggggccagagtcctttccaaggcttctccaattctgcccaaaaatatggttgatagtttacctgaataaatggtagtaatcacagttggccttcagaaccatccatagtagtatgatgatacaagattagaagctgaaaacctaagtcctttatgtggaaaacagaacatcattagaacaaaggacagagtatgaacacctgggcttaagaaatctagtatttcatgctgggaatgagacataggccatgaaaaaaatgatccccaagtgtgaacaaaagatgctcttctgtggaccactgcatgagcttttatactaccgacctggtttttaaatagagtttgctattagagcattgaattggagagaaggcctccctagccagcacttgtatatacgcatctataaattgtccgtgttcatacatttgaggggaaaacaccataaggtttcgtttctgtatacaaccctggcattatgtccactgtgtatagaagtagattaagagccatataagtttgaaggaaacagttaataccattttttaaggaaacaatataaccacaaagcacagtttgaacaaaatctcctcttttagctgatgaacttattctgtagattctgtggaacaagcctatcagcttcagaatggcattgtactcaatggatttgatgctgtttgacaaagttactgattcactgcatggctcccacaggagtgggaaaacactgccatcttagtttggattcttatgtagcaggaaataaagtataggtttagcctccttcgcaggcatgtcctggacaccgggccagtatctatatatgtgtatgtacgtttgtatgtgtgtagacaaatatttggaggggtatttttgccctgagtccaagagggtcctttagtacctgaaaagtaacttggctttcattattagtactgctcttgtttcttttcacatagctgtctagagtagcttaccagaagcttccatagtggtgcagaggaagtggaaggcatcagtccctatgtatttgcagttcacctgcacttaaggcactctgttatttagactcatcttactgtacctgttccttagaccttccataatgctactgtctcactgaaacatttaaattttaccctttagactgtagcctggatattattcttgtagtttacctctttaaaaacaaaacaaaacaaaacaaaaaactccccttcctcactgcccaatataaaaggcaaatgtgtacatggcagagtttgtgtgttgtcttgaaagattcaggtatgttgcctttatggtttcccccttctacatttcttagactacatttagagaactgtggccgttatctggaagtaaccatttgcactggagttctatgctctcgcacctttccaaagttaacagattttggggttgtgttgtcacccaagagattgttgtttgccatactttgtctgaaaaattcctttgtgtttctattgacttcaatgatagtaagaaaagtggttgttagttatagatgtctaggtacttcaggggcacttcattgagagttttgtcttgccatactttgtctgaaaaattcctttgtgtttctattgacttcaatgatagtaagaaaagtggttgttagttatagatgtctaggtacttcaggggcacttcattgagagttttgtcttggatattcttgaaagtttatatttttataattttttcttacatcagatgtttctttgcagtggcttaatgtttgaaattattttgtggctttttttgtaaatattgaaatgtagcaataatgtcttttgaatattcccaagcccatgagtccttgaaaatattttttatatatacagtaactttatgtgtaaatacataagcggcgtaagtttaaaggatgttggtgttccacgtgttttattcctgtatgttgtccaattgttgacagttctgaagaattctaataaaatgtacatatataaatcaa

SEQ ID NO: 2 - protein, 976 aa, Homo sapiens [sequence omitted]

(19) United States
(12) Patent Application Publication
(10) Pub. No.: US 2015/0297593 A1
(43) Pub. Date: Oct. 22, 2015

(54) INHIBITION OF VIRAL INFECTION-TRIGGERED ASTHMA WITH C-KIT INHIBITOR

(71) Applicants: CHILDREN'S MEDICAL CENTER CORPORATION, Boston, MA (US); The Regents of the University of California, Oakland, CA (US)

(72) Inventors: Dale T. Umetsu, Newton, MA (US); Ya-Jen Chang, Brookline, MA (US); Rosemarie H. De Kruyff, Newton, MA (US); Nicole Baumgarth, Davis, CA (US)

(21) Appl. No.: 14/428,272

(22) PCT Filed: Sep. 13, 2013
(86) PCT No.: PCT/US13/59609

§ 371 Date: Mar. 13, 2015

Related U.S. Application Data
(60) Provisional application No. 61/701,296, filed on Sep. 14, 2012.

(51) Int. Cl. A61K 31/506; A61K 39/395; A61K 45/06; A61K 31/496; C07K 16/28

(52) U.S. Cl. CPC A61K 31/506; A61K 31/496; C07K 16/2866; A61K 45/06; A61K 39/3955; C07K 2317/76

(57) ABSTRACT

The invention provides methods, compositions, and kits featuring a c-Kit kinase inhibitor for use in preventing or treating virus-induced respiratory pathology.

### RELATED APPLICATIONS

This application claims priority to U.S. Provisional Application No. 61/701,296, filed Sep. 14, 2012, incoporated by reference in its entirety herein.

### STATEMENT OF RIGHTS TO INVENTIONS MADE UNDER FEDERALLY SPONSORED RESEARCH

This work was supported by grant nos. R01 AI068085, RO1 HL62348, and RO1 051354 from the National Institutes of Health. The Government has certain rights in this invention.

### BACKGROUND OF THE INVENTION

Viral infection poses a major public health threat to individuals worldwide. For example, in the US alone, tens of millions of people develop the flu each year, resulting on average 40,000 deaths and more than 200,000 hospitalizations annually, with a total cost of over $10 billion/year. In 2009, during the swine flu II1N1 pandemic, the CDC estimated that between 43 million and 89 million cases of H1N1 infection occurred between April 2009 and April 2010 (infecting 14% to 30% of the population). Despite effective vaccines and anti-viral medications, hospitalization and serious illness due to influenza remain common, particularly in young children, the elderly, pregnant women, and in those with an underlying medical condition, including neurological conditions, heart disease, as well as respiratory disease and asthma. The lack of effective therapies for these patients is due in part to the lack of understanding of the immunological pathways that lead to virus-induced respiratory pathology.

The consequences of viral infection are particularly significant in individuals suffering from asthma, as demonstrated by the fact that wheezing was present in nearly half of all hospitalized patients with respiratory disorders during the 2009-2010 influenza A (H1N1) pandemic. Asthma itself is a major public health problem that affects nearly 10% of the general population. Asthma is characterized by airway inflammation, airway hyperreactivity (AHR), reversible airway obstruction and symptoms of wheezing and shortness of breath, and is thought to be mediated by allergen-specific Th2 cells, adaptive immunity and allergic inflammation. Current treatments for asthma are based on these ideas, and focus on minimizing Th2-driven eosinophilic airway inflammation, generally using anti-inflammatory therapies such as corticosteorids. However, this approach, targeting Th2 cell immunity, has not reduced the high rates of hospitalizations for acute asthma, most often caused by viral infection, including influenza. Because of this, asthma remains a very common medical diagnosis and is a primary reason for hospitalization, particularly in patients with virus infection.

Accordingly, there is a need for improving current methods for preventing and treating virus-induced respiratory pathology.

### SUMMARY OF THE INVENTION

As described below, the present invention features methods for treating virus-induced respiratory disease (e.g., virus-induced asthma). In embodiments, the invention features administering a c-Kit inhibitor to a subject at risk of viral infection or having a virus-induced respiratory pathology (e.g., influenza or respiratory syncytial virus-induced respiratory pathology) in an amount effective to treat a respiratory pathology.

In aspects, the invention provides methods for preventing or treating respiratory pathology in a subject. In embodiments, the methods involve administering an effective amount of a c-Kit kinase inhibitor to the subject.

In aspects, the invention provides methods for preventing or treating airway inflammation in a subject. In embodiments, the airway inflammation is associated with asthma. In embodiments, the methods involve administering an effective amount of a c-Kit kinase inhibitor to the subject.

In aspects, the invention provides methods for preventing or treating airway hyperreactivity (AHR) in a subject. In embodiments, the methods involve administering an effective amount of a c-Kit kinase inhibitor to the subject.

In aspects, the invention provides methods for preventing or treating acute asthma in a subject. In embodiments, the methods involve administering an effective amount of a c-Kit kinase inhibitor to the subject.

In any of the above aspects and embodiments, the subject has or is at risk of developing a viral infection.

In any of the above aspects and embodiments, administration of the c-Kit kinase inhibitor prevents or treats virus-induced respiratory pathology, virus-induced airway inflammation, or virus-induced AHR in the subject.

In aspects, the invention provides methods for preventing or treating virus-induced respiratory pathology in a subject. In embodiments, the methods involve administering an effective amount of a c-Kit kinase inhibitor to the subject.

In aspects, the invention provides methods for preventing or treating virus-induced airway inflammation in a subject. In embodiments, the methods involve administering an effective amount of a c-Kit kinase inhibitor to the subject.

In aspects, the invention provides methods for preventing or treating virus-induced airway hyperreactivity (AHR) in a subject. In embodiments, the methods involve administering an effective amount of a c-Kit kinase inhibitor to the subject.

In aspects, the invention provides methods for preventing or treating acute asthma in a subject. In embodiments, the methods involve administering an effective amount of a c-Kit kinase inhibitor to the subject.

In aspects, the invention provides methods for preventing or treating respiratory pathology in a subject. In embodiments, the methods involve administering to a subject having or at risk of developing a viral infection a c-Kit kinase inhibitor, thereby preventing or treating respiratory pathology in the subject.

In aspects, the invention provides methods for preventing or treating airway inflammation in a subject. In embodiments, the methods involve administering to a subject having or at risk of developing a viral infection a c-Kit kinase inhibitor, thereby preventing or treating airway inflammation in the subject.

In aspects, the invention provides methods for preventing or treating AHR in a subject. In embodiments, the methods involve administering to a subject having or at risk of developing a viral infection a c-Kit kinase inhibitor, thereby preventing or treating AHR in the subject.

In aspects, the invention provides methods for inhibiting nuocyte activation in a subject. In embodiments, the methods involve administering to a subject having or at risk of developing a viral infection a c-Kit kinase inhibitor, thereby inhibiting nuocyte activation in the subject.

In aspects, the invention provides methods for inhibiting lymphokine production in a subject. In embodiments, the methods involve administering to a subject having or at risk of developing a viral infection a c-Kit kinase inhibitor, thereby inhibiting lymphokine production in the subject. In related embodiments, the lymphokine can be IL-13 or IL-5.

In any of the above aspects, the virus can be any virus that causes asthma symptoms (e.g., respiratory syncytial virus (RSV), influenza, rhinovirus, parainfluenza, adenovirus, coronavirus, metapneumovirus, bocavirus, and the like).

In any of the above aspects, the viral infection can be infection by RSV, influenza, parainfluenza, adenovirus, coronavirus, metapneumovirus, or bocavirus.

In any of the above aspects, the subject can be a mammal In embodiments, the subject can be a human. In embodiments, the subject can be susceptible to viral infection. In some embodiments, the subject can be a pregnant female. In other embodiments, the subject can be a young subject (e.g., less than 10 years of age) or an infant subject. In yet other embodiments, the subject can be an elderly subject (e.g., at least 65 years old). In embodiments, the subject can have an underlying medical condition (e.g., a neurological condition, a heart condition, or a respiratory condition). In related embodiments, the underlying condition is asthma.

In any of the above aspects and embodiments, the subject does not respond to corticosteroid therapy.

In any of the above aspects and embodiments, the method reduces wheezing, shortness of breath, chest tightness, and coughing in the subject.

In any of the above aspects and embodiments, the viral infection can be an acute viral infection.

The c-Kit kinase inhibitor may also affect other cell types, including mast cells, eosinophils, but the major cell type that is required for airway hyperreactivity during viral infection is the nuocyte, also known as natural helper cells, innate type 2 cells, innate lymphoid cell 2, or multipotent progenitor cell.

In any of the above aspects and embodiments, the c-Kit kinase inhibitor can be administered to the subject for 1-7 days, 1-5 days, or 1-3 days.

In aspects, the invention provides methods for inhibiting nuocyte activation. In embodiments, the methods involve contacting a nuocyte with a c-Kit kinase inhibitor, thereby inhibiting nuocyte activation.

In aspects, the invention provides methods for inhibiting lymphokine production. In embodiments, the methods involve contacting a cell (e.g., a nuocyte) with a c-Kit kinase inhibitor, thereby inhibiting lymphokine production by the cell (e.g., a nuocyte).

In the above aspects, the cell/nuocyte can be contacted with the c-Kit kinase inhibitor for 1-7 days, 1-5 days, or 1-3 days.

In any of the above aspects and embodiments, the c-Kit kinase inhibitor can be a compound of Formula I, a compound of Formula II, dasatinib; imatinib; sunitinib; axitinib; pazopanib; cabozantinib; dovitinib; telatinib; Ki8751; OSI-930; AMN107; midostaurin; amuvatinib; tivozanib; regorafenib; vatalanib; masitinib; motesanib; or a salt, analog, or derivative thereof. In embodiments, the c-Kit kinase inhibitor is imatinib; masitinib; or a salt, analog, or derivative thereof.

In any of the above aspects and embodiments, the c-Kit kinase inhibitor can be an antibody or antibody fragment that selectively binds c-Kit. In embodiments, binding of the antibody or antibody fragment to c-Kit inhibits c-Kit kinase activity. In some embodiments, the antibody is a polyclonal antibody. In other embodiments, the antibody is a monoclonal antibody. In embodiments, the antibody or antibody fragment is humanized. In related embodiments, the antibody is a humanized monoclonal antibody.

In any of the above aspects and embodiments, the c-Kit kinase inhibitor can be an inhibitory nucleic acid molecule. In embodiments, the inhibitory nucleic acid molecule is an siRNA, shRNA or antisense nucleic acid molecule that reduces expression of c-Kit.

In any of the above aspects and embodiments, the method involves further administering at least one additional anti-asthma medication. In embodiments, the additional anti-asthma medication can be a corticosteroid, a beta-agonist, a leukotriene modifier, a mast cell stabilizer, theophylline, an immunomodulator, an anti-IgE therapy (e.g., omalizumab), or an anti-cholinergic. In related embodiments, the additional anti-asthma medication is a corticosteroid.

In aspects, the invention provides methods for preventing or treating influenza-induced AHR or airway inflammation in a subject. In embodiments, the methods involve administering an effective amount of a c-Kit kinase inhibitor to the subject.

In aspects, the invention provides methods for preventing or treating RSV-induced AIIR or airway inflammation in a subject. In embodiments, the methods involve administering an effective amount of a c-Kit kinase inhibitor to the subject.

In aspects, the invention provides methods for treating acute influenza infection in a subject. In embodiments, the methods involve administering an effective amount of a c-Kit kinase inhibitor to the subject to reduce AHR or airway inflammation in the subject.

In aspects, the invention provides methods for treating acute RSV infection in a subject. In embodiments, the methods involve administering an effective amount of a c-Kit kinase inhibitor to the subject to reduce AHR or airway inflammation in the subject.

In aspects, the invention provides methods for treating acute asthma in a subject. In embodiments, the methods involve administering an effective amount of a c-Kit kinase inhibitor to the subject to reduce AHR or airway inflammation in the subject. In embodiments, the acute asthma is caused by influenza or respiratory syncytial virus (RSV) infection.

In the above aspects, the c-Kit kinase inhibitor can be imatinib; masitinib; or a salt, analog, or derivative thereof.

In the above aspects, the c-Kit kinase inhibitor can be an antibody or antibody fragment that selectively binds c-Kit, wherein binding of the antibody or antibody fragment to c-Kit inhibits c-Kit kinase activity.

In the above aspects, the c-Kit kinase inhibitor can be administered to the subject for 1-3, 1-5, or 1-7 days.

In the above aspects, the method can involve further administering a corticosteroid.

In any of the above aspects and embodiments, the subject has acute asthma caused by viral infection.

In any of the above aspects and embodiments, the methods reduce wheezing, shortness of breath, chest tightness, and coughing in the subject.

In aspects, the invention provides a pharmaceutical composition containing a c-Kit kinase inhibitor for use in any of the methods contemplated herein. In embodiments, the pharmaceutical composition also contains a pharmaceutically acceptable carrier, diluent, or excipient.

In aspects, the invention provides kits for use in any of the methods contemplated herein. In embodiments, the kits are used for preventing or treating virus-induced respiratory pathology. In other embodiments, the kits are used for preventing or treating virus-induced airway inflammation or AHR. In yet other embodiments, the kits are used for inhibiting nuocyte activation or inhibiting lymphokine production.

In related embodiments, the kits contain a c-Kit kinase inhibitor.

In related embodiments, the kits contain directions for using the c-Kit kinase inhibitor in any of the methods contemplated herein.

In the above aspects and embodiments, the c-Kit kinase inhibitor can be a compound of Formula I, a compound of Formula II, dasatinib; imatinib; sunitinib; axitinib; pazopanib; cabozantinib; dovitinib; telatinib; Ki8751; OSI-930; AMN107; midostaurin; amuvatinib; tivozanib; regorafenib; vatalanib; masitinib; motesanib; or a salt, analog, or derivative thereof. In embodiments, the c-Kit kinase inhibitor is imatinib; masitinib; or a salt, analog, or derivative thereof.

In the above aspects and embodiments, the c-Kit kinase inhibitor can be an antibody or antibody fragment that selectively binds c-Kit. In embodiments, binding of the antibody or antibody fragment to c-Kit inhibits c-Kit kinase activity. In some embodiments, the antibody is a polyclonal antibody. In other embodiments, the antibody is a monoclonal antibody. In embodiments, the antibody or antibody fragment is humanized.

In any of the above aspects and embodiments, the c-Kit kinase inhibitor can be an inhibitory nucleic acid molecule. In embodiments, the inhibitory nucleic acid molecule is an siRNA, shRNA or antisense nucleic acid molecule that reduces expression of c-Kit.

In any of the above aspects and embodiments, the method involves further administering at least one additional anti-asthma medication. In embodiments, the additional anti-asthma medication can be a corticosteroid, a beta-agonist, a leukotriene modifier, a mast cell stabilizer, theophylline, an immunomodulator, an anti-IgE therapy (e.g., omalizumab), or an anti-cholinergic. In related embodiments, the additional anti-asthma medication is a corticosteroid.

In any of the above aspects or embodiments, an effective amount of a c-Kit kinase inhibitor is an amount sufficient to treat or prevent a virus-induced respiratory pathology.

Additional objects and advantages of the invention will be set forth in part in the description which follows, and in part will be obvious from the description, or may be learned by practice of the invention. The objects and advantages of the invention will be realized and attained by means of the elements and combinations disclosed herein, including those pointed out in the appended claims. It is to be understood that both the foregoing general description and the following detailed description are exemplary and explanatory only and are not restrictive of the invention as claimed. The accompanying drawings, which are incorporated herein and constitute a part of this specification, illustrate several embodiments of the invention and, together with the description, serve to explain the principles of the invention.

### Definitions

To facilitate an understanding of the present invention, a number of terms and phrases are defined below.

As used herein, the singular forms “a”, “an”, and “the” include plural forms unless the context clearly dictates otherwise. Thus, for example, reference to “a c-Kit kinase inhibitor” includes reference to more than one c-Kit kinase inhibitor.

Unless specifically stated or obvious from context, as used herein, the term “or” is understood to be inclusive.

As used herein, the terms “comprises,” “comprising,” “containing,” “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Administering” is defined herein as a means of providing an agent or a composition containing the agent to a subject in a manner that results in the agent being inside the subject's body. Such an administration can be by any route including, without limitation, oral, transdermal (e.g., vagina, rectum, oral mucosa), by injection (e.g., subcutaneous, intravenous, parenterally, intraperitoneally, intrathecal), or by inhalation (e.g., oral or nasal). Pharmaceutical preparations are, of course, given by forms suitable for each administration route.

By “agent” is meant any small molecule chemical compound, antibody, nucleic acid molecule, or polypeptide, or fragments thereof.

By “analog” is meant a molecule that is not identical, but has analogous functional or structural features. For example, an amide, ester, carbamate, carbonate, ureide, or phosphate analog of a c-Kit kinase inhibitor is a molecule that either: 1) does not destroy the biological activity of the c-Kit kinase inhibitor and confers upon that c-Kit kinase inhibitor advantageous properties in vivo, such as uptake, duration of action, or onset of action; or 2) is itself biologically inactive but is converted in vivo to a biologically active compound. Analogs include prodrug forms of a c-Kit kinase inhibitor. Such a prodrug is any compound that when administered to a biological system generates the c-Kit kinase inhibitor as a result of spontaneous chemical reaction(s), enzyme catalyzed chemical reaction(s), and/or metabolic chemical reaction(s).

The term “c-Kit” as used herein refers to a tyrosine kinase receptor activated by a stem cell factor ligand. Full nucleotide sequences encoding human c-Kit, and variants related thereto, are well-known in the art (see, e.g., NCBI Accession No. L04143, which is hereby incorporated by reference).

By “c-Kit nucleic acid molecule” and the like is meant a polynucleotide encoding a c-Kit polypeptide or fragment thereof.

By “c-Kit polypeptide” and the like is meant a protein or fragment thereof having at least 85%, 90%, 95%, 99%, or more identity to the amino acid sequence corresponding to NP_{—}000213, which is hereby incorporated by reference.

By “c-Kit kinase inhibitor” or “c-Kit inhibitor” is meant an agent that reduces the kinase activity of c-Kit. Reduction in kinase activity can be achieved by reducing the expression and/or the biological activity of c-Kit.

By “control” is meant a standard or reference condition.

The term “derivative” means a pharmaceutically active compound with equivalent or near equivalent physiological functionality to a given agent (e.g., a c-Kit kinase inhibitor). As used herein, the term “derivative” includes any pharmaceutically acceptable salt, ether, ester, prodrug, solvate, stereoisomer including enantiomer, diastereomer or stereoisomerically enriched or racemic mixture, and any other compound which upon administration to the recipient, is capable of providing (directly or indirectly) such a compound or an antivirally active metabolite or residue thereof.

By “disease” is meant any condition or disorder that damages or interferes with the normal function of a cell, tissue, or organ.

By “enhances” or “increases” is meant a positive alteration of at least about 10%, 25%, 50%, 75%, or 100% relative to a reference.

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen, or reversed Hoogsteen hydrogen bonding between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, or at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, HPLC analysis, and the like.

“Pharmaceutically acceptable” refers to approved or approvable by a regulatory agency of the Federal or a state government or listed in the U.S. Pharmacopeia or other generally recognized pharmacopeia for use in animals, including humans.

“Pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” of a c-Kit kinase inhibitor recited herein is an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4, and the like. Similarly, pharmaceutically acceptable cations include, but are not limited to sodium, potassium, calcium, aluminum, lithium and ammonium. Those of ordinary skill in the art will recognize further pharmaceutically acceptable salts for the c-Kit kinase inhibitors provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

The term “patient” or “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

As used herein, the terms “prevent,” “preventing,” “prevention,” “prophylactic treatment,” and the like, refer to reducing the probability of developing a disease or condition in a subject, who does not have, but is at risk of or susceptible to developing a disease or condition.

By “reduces” is meant a negative alteration of at least about 10%, 25%, 50%, 75%, or 100% relative to a reference.

By “reference” is meant a standard or control condition.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a polypeptide of the invention, but which does not substantially recognize and bind other molecules in a sample, for example, a biological sample, which naturally includes a polypeptide of the invention.

As used herein, the terms “treat,” treating,” “treatment,” and the like refer to reducing or ameliorating a disorder and/or symptoms associated therewith. By “ameliorate” is meant decrease, suppress, attenuate, diminish, arrest, or stabilize the development or progression of a disease. It will be appreciated that, although not precluded, treating a disorder or condition does not require that the disorder, condition or symptoms associated therewith be completely eliminated.

The term “therapeutic effect” refers to some extent of relief of one or more of the symptoms of a disorder or its associated pathology. In reference to the treatment of viral-induced respiratory disease (e.g., acute asthma and its associated pathologies, e.g., airway inflammation and airway hyperreactivity), a therapeutic effect refers to one or more of the following: 1) reducing, ameliorating, stopping, abating, alleviating, or inhibiting the symptoms of asthma, airway inflammation, or airway hyperreactivity (AHR), including wheezing, shortness of breath, chest tightness, and coughing; 2) controlling airway inflammation in order to reduce the reactivity of the airways and also to prevent airway remodeling such as permanent thickening of the bronchial walls as a result of chronic inflammation that does not resolve itself; and 3) prophylactically preventing the onset of symptoms in an individual at risk or diagnosed with virus-induced respiratory disease (e.g., acute asthma).

“Therapeutically effective amount” is intended to qualify the amount required to achieve a therapeutic effect. A physician or veterinarian having ordinary skill in the art can readily determine and prescribe the “therapeutically effective amount” (e.g., ED₅₀) of the pharmaceutical composition required. For example, the physician or veterinarian could start doses of the compounds of the invention employed in a pharmaceutical composition at levels lower than that required in order to achieve the desired therapeutic effect and gradually increase the dosage until the desired effect is achieved.

Typically a therapeutically effective dosage should produce a serum concentration of compound of from about 0.1 ng/ml to about 50-100 μg/ml. The pharmaceutical compositions typically should provide a dosage of from about 0.001 mg to about 2000 mg of compound per kilogram of body weight per day. For example, dosages for systemic administration to a human patient can range from 1-10 μg/kg, 20-80 μg/kg, 5-50 μg/kg, 75-150 μg/kg, 100-500 μg/kg, 250-750 μg/kg, 500-1000 μg/kg, 1-10 mg/kg, 5-50 mg/kg, 25-75 mg/kg, 50-100 mg/kg, 100-250 mg/kg, 50-100 mg/kg, 250-500 mg/kg, 500-750 mg/kg, 750-1000 mg/kg, 1000-1500 mg/kg, 1500-2000 mg/kg, 5 mg/kg, 20 mg/kg, 50 mg/kg, 100 mg/kg, 500 mg/kg, 1000 mg/kg, 1500 mg/kg, or 2000 mg/kg. Pharmaceutical dosage unit forms are prepared to provide from about 1 mg to about 5000 mg, for example from about 100 to about 2500 mg of the compound or a combination of essential ingredients per dosage unit form.

The phrase “combination therapy” embraces the administration of a c-Kit kinase inhibitor and a second therapeutic agent as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days, or weeks depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. For example, one combination of the present invention comprises a c-Kit kinase inhibitor and at least one additional therapeutic agent (e.g., corticosteroid) at the same or different times or they can be formulated as a single, co-formulated pharmaceutical composition comprising the two compounds. As another example, a combination of the present invention (e.g., a c-Kit kinase inhibitor and at least one additional therapeutic agent, such as a corticosteroid) is formulated as separate pharmaceutical compositions that can be administered at the same or different time. Sequential or substantially simultaneous administration of each therapeutic agent can be effected by any appropriate route including, but not limited to, oral routes, intravenous routes, intramuscular routes, and direct absorption through mucous membrane tissues (e.g., nasal, mouth, vaginal, and rectal). The therapeutic agents can be administered by the same route or by different routes. For example, one component of a particular combination may be administered by intravenous injection while the other component(s) of the combination may be administered orally. The components may be administered in any therapeutically effective sequence.

The phrase “combination” embraces groups of compounds or non-drug therapies useful as part of a combination therapy.

The term “virus-induced respiratory pathology” and the like refer to pulmonary pathology resulting from viral infection and virus-induced respiratory disease (e.g., acute asthma). Pulmonary pathology includes pathological conditions affecting organs and tissues that make gas exchange possible in mammals (e.g., humans). For example, pulmonary pathology refers to pathological conditions of the upper respiratory tract, trachea, bronchi, bronchioles, alveoli, pleura and pleural cavity, and the nerves and muscles of breathing.

Exemplary pulmonary pathology or virus-induced respiratory pathology include, but are not limited to, airway inflammation and AHR associated with acute asthma caused by viral infection (e.g., influenza, respiratory syncytial virus, rhinovirus, parainfluenza virus, adenovirus, coronavirus, metapneumovirus, or bocavirus infection).

As used herein, “reducing viral-induced respiratory pathology” and the like are meant to reduce the number and/or severity of pulmonary pathology (e.g., symptoms thereof) resulting from viral infection. In embodiments, the decrease is by at least 2-fold, 2.5-fold, 3-fold, 4-fold, 5-fold 10-fold, 20-fold, 30-fold, 40-fold, 50-fold, or more. In embodiments, reduction in viral-induced respiratory pathology results in the patient's pulmonary functions returning to baseline (e.g., prior to viral infection) or to control levels (e.g., a healthy subject's levels). In embodiments, reducing viral-induced respiratory pathology includes reducing wheezing, shortness of breath, chest tightness, and coughing in a subject.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless otherwise clear from the context, the term “alkyl” refers to a saturated aliphatic hydrocarbon radical including straight chain and branched chain groups of 1 to 20 carbon atoms (whenever a numerical range; e.g. “1-20”, is stated herein, it means that the group, in this case the alkyl group, may contain 1 carbon atom, 2 carbon atoms, 3 carbon atoms, etc. up to and including 20 carbon atoms). Alkyl groups containing from 1 to 4 carbon atoms are refered to as lower alkyl groups. When said lower alkyl groups lack substituents, they are referred to as unsubstituted lower alkyl groups. More preferably, an alkyl group is a medium size alkyl having 1 to 10 carbon atoms e.g., methyl, ethyl, propyl, 2-propyl, n-butyl, iso-butyl, tert-butyl, pentyl, and the like. Most preferably, it is a lower alkyl having 1 to 4 carbon atoms e.g., methyl, ethyl, propyl, 2-propyl, n-butyl, iso-butyl, or tert-butyl, and the like. The alkyl group may be substituted or unsubstituted. When substituted, the substituent group(s) is preferably one or more, more preferably one to three, even more preferably one or two substituent(s) independently selected from the group consisting of halo, hydroxy, unsubstituted lower alkoxy, aryl optionally substituted with one or more groups, preferably one, two or three groups which are independently of each other halo, hydroxy, unsubstituted lower alkyl or unsubstituted lower alkoxy groups, aryloxy optionally substituted with one or more groups, preferably one, two or three groups which are independently of each other halo, hydroxy, unsubstituted lower alkyl or unsubstituted lower alkoxy groups, 6-member heteroaryl having from 1 to 3 nitrogen atoms in the ring, the carbons in the ring being optionally substituted with one or more groups, preferably one, two or three groups which are independently of each other halo, hydroxy, unsubstituted lower alkyl or unsubstituted lower alkoxy groups, 5-member heteroaryl having from 1 to 3 heteroatoms selected from the group consisting of nitrogen, oxygen and sulfur, the carbon and the nitrogen atoms in the group being optionally substituted with one or more groups, preferably one, two or three groups which are independently of each other halo, hydroxy, unsubstituted lower alkyl or unsubstituted lower alkoxy groups, 5-or 6-member heteroalicyclic group having from 1 to 3 heteroatoms selected from the group consisting of nitrogen, oxygen and sulfur, the carbon and nitrogen (if present) atoms in the group being optionally substituted with one or more groups, preferably one, two or three groups which are independently of each other halo, hydroxy, unsubstituted lower alkyl or unsubstituted lower alkoxy groups, mercapto, (unsubstituted lower alkyl)thio, arylthio optionally substituted with one or more groups, preferably one, two or three groups which are independently of each other halo, hydroxy, unsubstituted lower alkyl or unsubstituted lower alkoxy groups, cyano, acyl, thioacyl, O-carbamyl, N-carbamyl, 0-thiocarbamyl, N-thiocarbamyl, C-amido, N-amido, nitro, N-sulfonamido, S-sulfonamido, R₁₈S(O)—, R₁₈S(O)₂—, —C(O)OR₁₈, R₁₈C(O)O—, and —NR₁₈ R₁₉, wherein R₁₈ and R₁₉ are independently selected from the group consisting of hydrogen, unsubstituted lower alkyl, trihalomethyl, unsubstituted (C₃-C₆)cycloalkyl, unsubstituted lower alkenyl, unsubstituted lower alkynyl and aryl optionally substituted with one or more, groups, preferably one, two or three groups which are independently of each other halo, hydroxy, unsubstituted lower alkyl or unsubstituted lower alkoxy groups.

Preferably, the alkyl group is substituted with one or two substituents independently selected from the group consisting of hydroxy, 5-or 6-member heteroalicyclic group having from 1 to 3 heteroatoms selected from the group consisting of nitrogen, oxygen and sulfur, the carbon and nitrogen (if present) atoms in the group being optionally substituted with one or more groups, preferably one, two or three groups which are independently of each other halo, hydroxy, unsubstituted lower alkyl or unsubstituted lower alkoxy groups, 5-member heteroaryl having from 1 to 3 heteroatoms selected from the group consisting of nitrogen, oxygen and sulfur, the carbon and the nitrogen atoms in the group being optionally substituted with one or more groups, preferably one, two or three groups which are independently of each other halo, hydroxy, unsubstituted lower alkyl or unsubstituted lower alkoxy groups, 6-member heteroaryl having from 1 to 3 nitrogen atoms in the ring, the carbons in the ring being optionally substituted with one or more groups, preferably one, two or three groups which are independently of each other halo, hydroxy, unsubstituted lower alkyl or unsubstituted lower alkoxy groups, or —NR₁₈ R₁₉, wherein R₁₈ and R₁₉ are independently selected from the group consisting of hydrogen, unsubstituted lower alkyl. Even more preferably the alkyl group is substituted with one or two substituents which are independently of each other hydroxy, dimethylamino, ethylamino, diethylamino, dipropylamino, pyrrolidino, piperidino, morpholino, piperazino, 4-lower alkylpiperazino, phenyl, imidazolyl, pyridinyl, pyridazinyl, pyrimidinyl, oxazolyl, triazinyl, and the like.

“Cycloalkyl” refers to a 3 to 8 member all-carbon monocyclic ring, an all-carbon 5-member/6-member or 6-member/6-member fused bicyclic ring or a multicyclic fused ring (a “fused” ring system means that each ring in the system shares an adjacent pair of carbon atoms with each other ring in the system) group wherein one or more of the rings may contain one or more double bonds but none of the rings has a completely conjugated pi-electron system.

Examples, without limitation, of cycloalkyl groups are cyclopropane, cyclobutane, cyclopentane, cyclopentene, cyclohexane, cyclohexadiene, adamantane, cycloheptane, cycloheptatriene, and the like. A cycloalkyl group may be substituted or unsubstituted. When substituted, the substituent group(s) is preferably one or more, more preferably one or two substituents, independently selected from the group consisting of unsubstituted lower alkyl, trihaloalkyl, halo, hydroxy, unsubstituted lower alkoxy, aryl optionally substituted with one or more, preferably one or two groups independently of each other halo, hydroxy, unsubstituted lower alkyl or unsubstituted lower alkoxy groups, aryloxy optionally substituted with one or more, preferably one or two groups independently of each other halo, hydroxy, unsubstituted lower alkyl or unsubstituted lower alkoxy groups, 6-member heteroaryl having from 1 to 3 nitrogen atoms in the ring, the carbons in the ring being optionally substituted with one or more, preferably one or two groups independently of each other halo, hydroxy, unsubstituted lower alkyl or unsubstituted lower alkoxy groups, 5-member heteroaryl having from 1 to 3 heteroatoms selected from the group consisting of nitrogen, oxygen and sulfur, the carbon and nitrogen atoms of the group being optionally substituted with one or more, preferably one or two groups independently of each other halo, hydroxy, unsubstituted lower alkyl or unsubstituted lower alkoxy groups, 5-or 6-member heteroalicyclic group having from 1 to 3 heteroatoms selected from the group consisting of nitrogen, oxygen and sulfur, the carbon and nitogen (if present) atoms in the group being optionally substituted with one or more, preferably one or two groups independently of each other halo, hydroxy, unsubstituted lower alkyl or unsubstituted lower alkoxy groups, mercapto,(unsubstituted lower alkyl)thio, arylthio optionally substituted with one or more, preferably one or two groups independently of each other halo, hydroxy, unsubstituted lower alkyl or unsubstituted lower alkoxy groups, cyano, acyl, thioacyl, O-carbamyl, N-carbamyl, O-thiocarbamyl, N-thiocarbamyl, C-amido, N-amido, nitro, N-sulfonamido, S-sulfonamido, R₁₈ S(O)—, R₁₈S(O)₂—, —C(O)OR₁₈, R₁₈ C(O)O—, and —NR₁₈ R₁₉ are as defined above.

“Alkenyl” refers to a lower alkyl group, as defined herein, consisting of at least two carbon atoms and at least one carbon-carbon double bond. Representative examples include, but are not limited to, ethenyl, 1-propenyl, 2-propenyl, 1-, 2-, or 3-butenyl, and the like.

“Alkynyl” refers to a lower alkyl group, as defined herein, consisting of at least two carbon atoms and at least one carbon-carbon triple bond. Representative examples include, but are not limited to, ethynyl, 1-propynyl, 2-propynyl, 1-, 2-, or 3-butynyl, and the like.

“Aryl” refers to an all-carbon monocyclic or fused-ring polycyclic (i.e., rings which share adjacent pairs of carbon atoms) groups of 1 to 12 carbon atoms having a completely conjugated pi-electron system. Examples, without limitation, of aryl groups are phenyl, naphthalenyl and anthracenyl. The aryl group may be substituted or unsubstituted. When substituted, the substituted group(s) is preferably one or more, more preferably one, two or three, even more preferably one or two, independently selected from the group consisting of unsubstituted lower alkyl, trihaloalkyl, halo, hydroxy, unsubstituted lower alkoxy, mercapto,(unsubstituted lower alkyl)thio, cyano, acyl, thioacyl, O-carbamyl, N-carbamyl, O-thiocarbamyl, N-thiocarbamyl, C-amido, N-amido, nitro, N-sulfonamido, S-sulfonamido, R₁₈S(O)—, R₁₈S(O)₂—, —C(O)OR₁₈, R₁₈ C(O)O—, and —NR₁₈R₁₉, wherein R₁₈ and R₁₉ are as defined above. Preferably, the aryl group is optionally substituted with one or two substituents independently selected from halo, unsubstituted lower alkyl, trihaloalkyl, hydroxy, mercapto, cyano, N-amido, mono or dialkylamino, carboxy, or N-sulfonamido.

“Heteroaryl” refers to a monocyclic or fused ring (i.e., rings which share an adjacent pair of atoms) group of 5 to 12 ring atoms containing one, two, or three ring heteroatoms selected from N, O, or S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, isoquinoline, purine and carbazole. The heteroaryl group may be substituted or unsubstituted. When substituted, the substituted group(s) is preferably one or more, more preferably one, two, or three, even more preferably one or two, independently selected from the group consisting of unsubstituted lower alkyl, trihaloalkyl, halo, hydroxy, unsubstituted lower alkoxy, mercapto,(unsubstituted lower alkyl)thio, cyano, acyl, thioacyl, O-carbamyl, N-carbamyl, O-thiocarbamyl, N-thiocarbamyl, C-amido, N-amido, nitro, N-sulfonamido, S-sulfonamido, R₁₈S(O)—, R₁₈O)₂—, —C(O)OR₁₈, R₁₈C(O)O—, and —NR₁₈R₁₉, with R₁₈ and R₁₉ as defined above. Preferably, the heteroaryl group is optionally substituted with one or two substituents independently selected from halo, unsubstituted lower alkyl, trihaloalkyl, hydroxy, mercapto, cyano, N-amido, mono or dialkylamino, carboxy, or N-sulfonamido.

“Heteroalicyclic” refers to a monocyclic or fused ring group having in the ring(s) of 5 to 9 ring atoms in which one or two ring atoms are heteroatoms selected from N, O, or S(O)ₙ (where n is an integer from 0 to 2), the remaining ring atoms being C. The rings may also have one or more double bonds. However, the rings do not have a completely conjugated pi-electron system. Examples, without limitation, of unsubstituted heteroalicyclic groups are pyrrolidino, piperidino, piperazino, morpholino, thiomorpholino, homopiperazino, and the like. The heteroalicyclic ring may be substituted or unsubstituted. When substituted, the substituted group(s) is preferably one or more, more preferably one, two or three, even more preferably one or two, independently selected from the group consisting of unsubstituted lower alkyl, trihaloalkyl, halo, hydroxy, unsubstituted lower alkoxy, mercapto,(unsubstituted lower alkyl)thio, cyano, acyl, thioacyl, O-carbamyl, N-carbamyl, O-thiocarbamyl, N-thiocarbamyl, C-amido, N-amido, nitro, N-sulfonamido, S-sulfonamido, R₁₈S(O)—, R₁₈S(O)₂—, —C(O)OR₁₈, R₁₈C(O)O—, and —NR₁₈R₁₉, with R₁₈ and R₁₉ as defined above. Preferably, the heteroalicyclic group is optionally substituted with one or two substituents independently selected from halo, unsubstituted lower alkyl, trihaloalkyl, hydroxy, mercapto, cyano, N-amido, mono or dialkylamino, carboxy, or N-sulfonamido.

Preferably, the heteroalicyclic group is optionally substituted with one or two substituents independently selected from halo, unsubstituted lower alkyl, trihaloalkyl, hydroxy, mercapto, cyano, N-amido, mono or dialkylamino, carboxy, or N-sulfonamido.

“Heterocycle” means a saturated cyclic radical of 3 to 8 ring atoms in which one or two ring atoms are heteroatoms selected from N, O, or S(O)ₙ (where n is an integer from 0 to 2), the remaining ring atoms being C, where one or two C atoms may optionally be replaced by a carbonyl group. The heterocyclyl ring may be optionally substituted independently with one, two, or three substituents selected from optionally substituted lower alkyl (substituted with 1 or 2 substituents independently selected from carboxy or ester), haloalkyl, cyanoalkyl, halo, nitro, cyano, hydroxy, alkoxy, amino, monoalkylamino, dialkylamino, aralkyl, heteroaralkyl, —COR (where R is alkyl) or —COOR where R is (hydrogen or alkyl). More specifically the term heterocyclyl includes, but is not limited to, tetrahydropyranyl, 2,2-dimethyl-1,3-dioxolane, piperidino, N-methylpiperidin-3-yl, piperazino, N-methylpyrrolidin 3-yl, 3-pyrrolidino, morpholino, thiomorpholino, thiomorpholino-1-oxide, thiomorpholino 1,1-dioxide, 4-ethyloxycarbonylpiperazino, 3-oxopiperazino, 2-imidazolidone, 2-pyrrolidinone, 2-oxohomopiperazino, tetrahydropyrimidin-2-one, and the derivatives thereof. Preferably, the heterocycle group is optionally substituted with one or two substituents independently selected from halo, unsubstituted lower alkyl, lower alkyl substituted with carboxy, ester, hydroxy, mono or dialkylamino.

“Hydroxy” refers to an —OH group.

“Alkoxy” refers to both an —O-(unsubstituted alkyl) and an —O-(unsubstituted cycloalkyl) group. Representative examples include, but are not limited to, e.g., methoxy, ethoxy, propoxy, butoxy, cyclopropyloxy, cyclobutyloxy, cyclopentyloxy, cyclohexyloxy, and the like.

“Aryloxy” refers to both an —O-aryl and an —O-heteroaryl group, as defined herein. Representative examples include, but are not limited to, phenoxy, pyridinyloxy, furanyloxy, thienyloxy, pyrimidinyloxy, pyrazinyloxy, and the like, and derivatives thereof.

“Mercapto” refers to an —SH group.

“Alkylthio” refers to both an —S-(unsubstituted alkyl) and an —S-(unsubstituted cycloalkyl) group. Representative examples include, but are not limited to, e.g., methylthio, ethylthio, propylthio, butylthio, cyclopropylthio, cyclobutylthio, cyclopentylthio, cyclohexylthio, and the like.

“Arylthio” refers to both an —S—aryl and an —S—heteroaryl group, as defined herein. Representative examples include, but are not limited to, phenylthio, pyridinylthio, furanylthio, thientylthio, pyrimidinylthio, and the like and derivatives thereof.

“Acyl” refers to a —C(O)—R” group, where R” is selected from the group consisting of hydrogen, unsubstituted lower alkyl, trihalomethyl, unsubstituted cycloalkyl, aryl optionally substituted with one or more, preferably one, two, or three substituents selected from the group consisting of unsubstituted lower alkyl, trihalomethyl, unsubstituted lower alkoxy, halo and —NR₁₈R₁₉ groups, heteroaryl (bonded through a ring carbon) optionally substituted with one or more, preferably one, two, or three substitutents selected from the group consisting of unsubstituted lower alkyl, trihaloalkyl, unsubstituted lower alkoxy, halo and —NR₁₈R₁₉ groups and heteroalicyclic (bonded through a ring carbon) optionally substituted with one or more, preferably one, two, or three substituents selected from the group consisting of unsubstituted lower alkyl, trihaloalkyl, unsubstituted lower alkoxy, halo and —NR₁₈R₁₉ groups. Representative acy groups include, but are not limited to, acetyl, trifluoroacetyl, benzoyl, and the like.

“Aldehyde” refers to an acyl group in which R″ is hydrogen.

“Thioacyl” refers to a —C(S)—R″ group, with R″ as defined herein.

“Ester” refers to a —C(O)—R″ group with R″ as defined herein except that R″ cannot be hydrogen.

“Acetyl” group refers to a —C(O)CH₃ group.

“Halo” group refers to fluorine, chlorine, bromine or iodine, preferably fluorine or chlorine.

“Trihalomethyl” group refers to a —CX₃ group wherein X is a halo group as defined herein.

“Trihalomethanesulfonyl” group refers to a X₃ CS(═O)₂-groups with X as defined above.

“Cyano” refers to a —CN group.

“Methylenedioxy” refers to a —OCH₂O— group where the two oxygen atoms are bonded to adjacent carbon atoms.

“Ethylenedioxy” group refers to a —OCH₂CH₂O— where the two oxygen atoms are bonded to adjacent carbon atoms.

“S-sulfonamido” refers to a —S(O)₂NR₁₈R₁₉ group, with R₁₈ and R₁₉ as defined herein.

“N-sulfonamido” refers to a —NR₁₈S(O)₂R₁₉ group, with R₁₈ and R₁₉ as defined herein.

“O-carbamyl” group refers to a —OC(O)NR₁₈ R₁₉ group with R₁₈ and R₁₉ as defined herein.

“N-carbamyl” refers to an R₁₈OC(O)NR₁₉-group, with R₁₈ and R₁₉ as defined herein.

“O-thiocarbamyl” refers to a —OC(S)NR₁₈R₁₉ group with R₁₈ and R₁₉ as defined herein.

“N-thiocarbamyl” refers to a R₁₈ OC(S)NR₁₉-group, with R₁₈ and R₁₉ as defined herein.

“Amino” refers to an —NR₁₈R₁₉ group, wherein R₁₈ and R₁₉ are both hydrogen.

“C-amido” refers to a —C(O)NR₁₈ R₁₉ group with R₁₈ and R₁₉ as defined herein.

“N-amido” refers to a R₁₈ C(O)NR₁₉-group, with R₁₈ and R₁₉ as defined herein.

“Nitro” refers to a —NO₂ group.

“Haloalkyl” means an unsubstituted alkyl, preferably unsubstituted lower alkyl as defined above that is substituted with one or more same or different halo atoms, e.g., —CH₂Cl, —CF₃, —CH₂CF₃, —CH₂CCl₃, and the like.

“Aralkyl” means unsubstituted alkyl, preferably unsubstituted lower alkyl as defined above which is substituted with an aryl group as defined above, e.g., —CH₂ phenyl, —(CH₂)₂ phenyl, —(CH₂)₃ phenyl, CH₃CH(CH₃)CH₂ phenyl, and the like and derivatives thereof.

“Heteroaralkyl” group means unsubstituted alkyl, preferably unsubstituted lower alkyl as defined above which is substituted with a heteroaryl group, e.g., —CH₂ pyridinyl, —(CH₂)₂ pyrimidinyl, —(CH₂)₃ imidazolyl, and the like, and derivatives thereof.

“Monoalkylamino” means a radical —NHR where R is an unsubstitued alkyl or unsubstituted cycloalkyl group as defined above, e.g., methylamino, (1-methylethyl)amino, cyclohexylamino, and the like.

“Dialkylamino” means a radical —NRR where each R is independently an unsubstitued alkyl or unsubstituted cycloalkyl group as defined above, e.g., dimethylamino, diethylamino, (1-methylethyl)-ethylamino, cyclohexylmethylamino, cyclopentylmethylamino, and the like.

“Cyanoalkyl” means unsubstituted alkyl, preferably unsubstituted lower alkyl as defined above, which is substituted with 1 or 2 cyano groups.

“Optional” or “optionally” means that the subsequently described event or circumstance may but need not occur, and that the description includes instances where the event or circumstance occurs and instances in which it does not. For example, “heterocycle group optionally substituted with an alkyl group” means that the alkyl may but need not be present, and the description includes situations where the heterocycle group is substituted with an alkyl group and situations where the heterocyclo group is not substituted with the alkyl group.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### DESCRIPTION OF THE DRAWINGS

FIGS. 1A-1G show that imatinib abolishes II3N1 infection-induced airway hyperreactivity (AHR) and inflammation. In FIGS. 1A and 1B, 8 wk old BALB/c mice (a-b, n=10 per group) were treated with imatinib (oral administration; 50 mg/kg on day-1, day 1 and day 4), infected with influenza A virus (Mem71, H3N1) or control allantoic fluid (mock-), and assessed 5 days post-infection for AHR. In FIGS. 1C and 1D, 8 wk old BALB/c mice (a-b, n=10 per group) were treated with imatinib (i.p. administration; 100 mg/kg on day 1 and day 4) or dexamethasone (i.p. administration; 100 ug/mouse on day 1 and day 4), infected with influenza A virus or control allantoic fluid (mock-infection), and assessed 5 days post-infection for AHR. In FIGS. 1E and 1F, 8 wk old BALB/c mice (a-b, n=10 per group) were treated with imatinib (i.p. administration; 100 mg/kg on day 1) or dexamethasone (i.p. administration; 100 ug/mouse on day 1), infected with influenza A virus (Mem71, H3N1) or control allantoic fluid, and assessed 5 days post-infection for AHR. FIGS. 1A, 1C, and 1E are graphs depicting the changes in lung resistance (R_{L}) in anesthetized, tracheotomized, intubated, and mechanically ventilated mice. *p<0.001 compared to H3N1-infected group. FIGS. 1B, 1D, and 1F are graphs showing the number of macrophage (Mac), neutrophil (Neu), eosinophil (Eos) and lymphocyte (Lym) present in bronchoalveolar (BAL) fluid, which was collected and analyzed 5 days after the virus challenge. n.d., not detectable; *p<0.001 compared to H3N1-infected group. n.s., not significant. FIG. 1G includes representative lung sections from mock or H3N1-infected BALB/c, treated with imatinib (100 mg/kg on day 1), stained with hematoxylin and eosin, and assessed 5 days post-infection. Scale bars, 200 μm. Data are representative of 3 independent experiments.

FIGS. 2A-2C are schematic drawings that show the imatinib treatment protocols for evaluating influenza induced AHR and lung inflammation. FIG. 2A shows the protocol used in the experiments for FIGS. 1A and 1B (50 mg/kg; oral gavage administration). FIG. 2B shows the protocol used in the experiments for FIGS. 1C and 1D (100 mg/kg; intraperitoneal administration). FIG. 2C shows the protocol used in the experiments for FIGS. 1E-1G (100 mg/kg; intraperitoneal administration).

FIGS. 3A-3D show that dexamethasone abolishes OVA-induced but not H3N1 infection-caused AHR and inflammation. In FIGS. 3A and 3B, 8 wk old BALB/c mice (a-b, n=5 per group) were administered OVA/Alum on day 0 (i.p. administration) and treated with or without dexamethasone (i.p. administration; 100 ug/mouse on day 7, 8, 9) and with OVA in challenge for 3 consecutive days (days 7, 8, 9). AHR was assessed on the day after last OVA-challenge. Control mice received i.p. injection of PBS and intranasal administrations of normal saline. In FIGS. 3C and 3D, 8 wk old BALB/c mice (a-b, n=10 per group) were treated with dexamethasone (i.p. administration; 100 ug/mouse on day-1, day 1 and day 4), infected with influenza A virus (Mem71, H3N1) or control allantoic fluid (mock-infection), and assessed 5 days post-infection for AHR. FIGS. 3A and 3C are graphs depicting the changes in lung resistance (R_{L}) in anesthetized, tracheotomized, intubated, and mechanically ventilated mice. *p<0.001 compared to H3N1-infected group. FIGS. 3B and 3C are graphs showing the number of macrophage (Mac), neutrophil (Neu), eosinophil (Eos) and lymphocyte (Lym) present in bronchoalveolar (BAL) fluid, which was collected and analyzed 24 hr after last OVA-challenge, or 5 days after the virus challenge. n.d., not detectable; *p<0.001 compared to H3N1-infected group. Data are representative of 3 independent experiments.

FIGS. 4A and 4B show that imatinib inhibits H3N1 infection-induced AHR and inflammation in Rag2−/− mice. 8 wk old Rag2−/− mice (a-b, n=5 per group) were treated with imatinib (i.p. administration; 100 mg/kg on day 1), infected with influenza A virus (Mem71, H3N1) or control allantoic fluid (mock-infection), and assessed 5 days post-infection for AHR. FIG. 4A is a graph depicting the changes in lung resistance (R_{L}) in anesthetized, tracheotomized, intubated, and mechanically ventilated mice. *p<0.001 compared to H3N1-infected group. FIG. 4B is a graph showing the number of macrophage (Mac), neutrophil (Neu), eosinophil (Eos) and lymphocyte (Lym) present in bronchoalveolar (BAL) fluid, which was collected and analyzed 5 days after the virus challenge. n.d., not detectable; *p<0.001 compared to H3N1-infected group. Data are representative of 3 independent experiments.

FIGS. 5A-5D show that imatinib inhibits IL-33-induced AHR and nuocyte proliferation in Rag2^{-/-} mice. 8 wk old Rag2^{-/-} mice (a-b, n=5 per group) received IL-33 (1 μg, i.n., day 0), were treated with imatinib (i.p. administration; 100 mg/kg on day 1), and were assessed on day 5 for AHR. FIG. 5A depicts the change in lung resistance (R_{L}) in anesthetized, tracheotomized, intubated, and mechanically ventilated mice. *p<0.001 compared to IL-33 treated Rag2^{-/-} group. FIG. 5B shows the number of macrophage (Mac), neutrophil (Neu), eosinophil (Eos) and lymphocyte (Lym) present in bronchoalveolar (BAL) fluid, which was collected and analyzed 5 days after received IL-33. *p<0.001 compared to IL-33 treated Rag2^{-/-} group. Data are representative of 3 independent experiments. In FIGS. 5C and 5D, BAL cells (FIG. 5C) or lung cells (FIG. 5D) were taken from 8 wk old Rag2^{-/-} mice (n=5 per group) on day 5, and further stimulated with PMA+ionomycin for 5 hr. The percentage of lung CD45⁺Lin⁻ST2⁺ckit⁺Sca1⁺ cells was assessed by FACS. Third panels show dot plots for Lin⁻ST2⁺ cells in lung leukocytes (CD45⁺). After gating on the Lin⁻ST2⁺cKit⁺ cells, the cells were analyzed for c-Kit and Sca1 expression (fourth panels).

FIGS. 6A-6F show that imatinib inhibits the increase in lung nuocytes induced by influenza virus infection. In FIGS. 6A-6C, 8 wk-old BALB/c mice were infected with H3N1 or mock infected with allantoic fluid, and treated with Imatinib (100mg/kg) on day 1. The BAL fluid cells (FIG. 6A) or lung cells (FIG. 6B) were isolated and assessed day 5 post-infection. The frequency of Lin⁻ST2⁺ cells in CD45⁺ cells was assessed by FACS (first and second panels). After gating on the Lin⁻ST2⁺ subset (third panels), the cells from H3N1 or mock-infected mice were further analyzed for Sca-1 and c-Kit expression (fourth panels). In FIG. 6C, the absolute numbers of CD45⁺Lin⁻ST2⁺c-Kit⁺Sca1⁺ cells in BAL (left panel), and lung (right panel) were assessed by FACS. *p<0.001 compared to H3N1-infected group. Data are representative of 3 independent experiments. *p<0.001 compared to H3N1-infected group. In FIGS. 6D-6F, 8 wk-old BALB/c mice were infected with H3N1 or mock infected with allantoic fluid, and treated with imatinib (100 mg/kg) on day 1. The lung cells (FIG. 6D) were isolated and analyzed for NKT cells by FACS day 5 post-infection. The third row panels show the dot plots and percentage of NKT (CD 1d tetramer⁺TCRβ⁺) and conventional T cells (CD1d tetramer-TCRβ⁺) in CD45⁺ leukocytes (first row panels). After gating on the NKT cells or T cells, the cells were analyzed for CD4 and CD8 (fourth or fifth row panels, respectively). The absolute numbers of lung CD1d tetra⁺TCRβ⁺ NKT cells (FIG. 6E), and lung CD1d tetra⁻TCRβ⁺ T cells, including TCRβ⁺CD4⁺ T cells and TCRβ⁺CD8⁺ T cells (FIG. 6F) were assessed by FACS. *p<0.001 compared to H3N1-infected group. Data are representative of 3 independent experiments.

FIGS. 7A-7D show that imatinib inhibits mouse nuocyte proliferation and cytokine productions. In FIG. 7A, lung nuocytes (Lin⁻ST2⁺) were isolated from the Rag2^{-/-} mice that received IL-33 (1 μg i.n.). FIG. 7A is a plot showing the purity of Lin⁻ST2⁺ cells as assessed by FACS after sorting. In FIGS. 7B-7D, lung nuocytes (Lin⁻ST2⁺) (4×10⁴ cells/well, 96 well plates) were cultured with 50 ng/ml IL-2, or plus 100 ng/ml IL-33 for 24 hrs with or without imatinib (0.1-1 μM) in vitro. FIG. 7B is a graph showing thymidine incorporation of nuocytes assessed on day 6. FIGS. 7C and 7D are graphs showing the results from supernatants collected on day 6 from triplicate wells that were assessed for IL-5 and IL-13 protein by ELISA. *p<0.01 and **p<0.001 compared to IL2/IL33 group.

FIGS. 8A-8C shows that imatinib inhibits human nuocyte IL-13 secretion in vitro. In FIG. 8A, human alveolar macrophages (AM) were infected with H3N1 (M.O.I=5) or with H1N1 (M.O.I=1) for 24 hr. Total RNA from the cells was analyzed by qRT-PCR for IL-33 mRNA expression. *p<0.001, compared to mock-infected group. Data are representative of 3 independent experiments. In FIG. 8B, human BAL cells (left panels) or PBMC (right panels) were isolated and cultured with 50 ng/ml IL-2 plus 100 ng/ml IL-33 for 24 hrs with or without imatinib (1 uM) in vitro. After 24 hrs, the cells were further stimulated with PMA+ionomycin for 5 hr. The percentage of lung CD45⁺Lin⁻cKit⁺ cells was assessed by FACS. The third row panels show dot plots for Lin⁻cKit⁺ cells in human leukocytes (CD45⁺). After gating on the Lin⁻cKit⁺ cells, the cells were analyzed for intracellular IL-13, CD127 (IL-7R) and CD34 (marker of human nuocytes) expression (fourth and fifth row panels, respectively). In FIG. 8C, PBMC from individual donor (n=5) were isolated and cultured with 50 ng/ml IL-2 plus 100 ng/ml IL-33 for 24 hrs with or without imatinib (1 uM) in vitro. Quantitative data are shown.

FIGS. 9A-9D show that masitinib abolishes H3N1 infection-caused AHR and inflammation. In FIGS. 9A and 9B, 8 wk old BALB/c mice (a-b, n=10 per group) were treated with mastinib (500 ug/mouse on day 1), infected with influenza A virus (Mem71, H3N1) or control allantoic fluid (mock-infection), and assessed 5 days post-infection for AHR. In FIGS. 9C and 9D, 8 wk old BALB/c mice (a-b, n=10 per group) were treated with anti-cKit mAb or control IgG (250 ug/mouse on day 1), infected with influenza A virus (Mem71, H3N1) or control allantoic fluid (mock-infection), and assessed 5 days post-infection for AHR. FIGS. 9A and 9C are graphs depicting the changes in lung resistance (R_{L}) in anesthetized, tracheotomized, intubated, and mechanically ventilated mice. *p<0.001 compared to H3N1-infected group. FIGS. 9B and 9D are graphs showing the number of macrophage (Mac), neutrophil (Neu), eosinophil (Eos) and lymphocyte (Lym) present in bronchoalveolar (BAL) fluid, which was collected and analyzed 5 days after the virus challenge. n.d., not detectable; *p<0.001 compared to H3N1-infected group. Data are representative of 3 independent experiments.

FIGS. 10A-10C show that imatinib does not affect viral clearance and does not cause bone marrow suppression. In FIG. 10A, the lungs of H3N1-infected 8 wk old BALB/c mice (n=3-8 per group) with treatment of imatinib (100 mg/kg on day 1) were taken on the indicated days after infection and assessed for influenza virus by qRT-PCR. The data are presented as relative PFU/lung on a log scale.. In FIG. 10B, the sera of H3N1-infected 8 wk old BALB/c mice (n=3 per group) with i.p. administration of imatinib (100 mg/kg on day 1) were taken on day 11 after infection and assessed for level of antibody against H3N1 by ELISA. In FIG. 10C, the blood of 8 wk old BALB/c mice (n=5 per group) with i.p. administration of Imatinib (100 mg/kg on day 1) were taken before treatment (pre-treat) or 5 days after treatment (Imatinib) for the complete blood cell count (CBC) test. The counts of HGB, PLT, white blood cells (WBC), granulocyte, and lymphocyte, were shown. n.s., not significant.

FIGS. 11A-11G show that imatinib treatment abolishes RSV-induced AHR. In FIGS. 11A and 11B8 wk old BALB/c mice (a-b, n=10 per group), infected with RSV virus or control sham fluid (mock-infection), were assessed 6 days post-infection for AHR. FIG. 11A is a graph depicting the changes in lung resistance (R_{L}) measured in anesthetized, tracheotomized, intubated, and mechanically ventilated mice. *p<0.001 compared to RSV-infected group. In FIG. 11B, cells in bronchoalveolar (BAL) fluid were collected and the numbers of macrophage (Mac), neutrophil (Neu), eosinophil (Eos) and lymphocyte (Lym) were analyzed 6 days after the virus challenge. In FIGS. 11C-11E, 8 wk-old BALB/c mice were infected with RSV virus or control sham fluid (mock-infection), and the BAL fluid cells (FIG. 11C) or lung cells (FIG. 11D) were isolated and assessed day 6 post-infection. The frequency of Lin⁻ST2⁺ cells in CD45⁺ cells was assessed by FACS (first and second panels). After gating on the Lin⁻ST2⁺ subset (third panels), the cells from RSV or mock-infected mice were further analyzed for Sca-1 and c-Kit expression (fourth panels). In FIG. 11E, the absolute numbers of CD45⁺lin⁻ST2⁺c-Kit⁺Sca-1⁺ cells in BAL (upper panel), and lung (lower panel) were assessed by FACS. *p<0.001 compared to H3N1-infected group. Data are representative of 3 independent experiments. *p<0.001 compared to H3N1-infected group. In FIGS. 11F and 11G, 8 wk old BALB/c mice (a-b, n=5 per group), treated with imatinib (i.p. administration; 100 mg/kg on day 1) and infected with RSV virus or control sham fluid (mock-infection), were assessed 6 days post-infection for AHR. FIG. 11F is a graph showing the changes in lung resistance (R_{L}) measured in anesthetized, tracheotomized, intubated, and mechanically ventilated mice. *p<0.001 compared to RSV-infected group. In FIG. 11G, cells in bronchoalveolar (BAL) fluid were collected and the numbers of macrophage (Mac), neutrophil (Neu), eosinophil (Eos) and lymphocyte (Lym) were analyzed 5 days after the virus challenge. n.d., not detectable; *p<0.001 compared to H3N1-infected group. n.s., not significant.

FIGS. 12A-12H show that RSV-induced AHR requires an IL-33-ST2 axis and nuocytes. In FIGS. 12A and 12B, 8wk old heterozygous littermate, or homozygous Il1rl1 (St2)^{-/-} KO mice (n=4-6 per group), treated with RSV virus or control sham fluid (mock-infection), were assessed 6 days post-infection for AHR. FIG. 12A is graph showing the changes in lung resistance (R_{L}) in response to methacholine. In FIG. 12B, total cell numbers in BAL fluid were enumerated 6 days after the virus challenge. FIG. 12C is a graph showing that infection with RSV induces IL-33 production in alveolar macrophages (AM), interstitial macrophages (IM), dendritic cells (DC) and airway epithelial cells (CD45− cells), as assessed by intracellular cytokine staining. Lung cells were taken from RSV or mock infected BALB/c mice on day 1. Alveolar macrophages (F4/80⁺CD11c⁺), interstitial macrophages (F4/80⁺CD11c⁻) and dendritic cells (F4/80⁻ CD11c⁺) (CD45⁺) and airway epithelial cells (CD45⁻) were assessed for IL-33 production by flow cytometry. In FIG. 12D, human lung epithelial cells (A549 cell line) were infected with RSV (M.O.I=1 or 5), or sham control for 24 hr in vitro. Total RNA from the cells was analyzed by qRT-PCR for IL-33 mRNA expression. *p<0.001, compared to mock-infected group. Data are representative of three independent experiments. In FIGS. 12E and 12F, 8 wk old WT or Il13^{-/-} mice were infected with RSV or control sham fluid (mock-infection), and analyzed for AHR (FIG. 12E) and cells in BAL fluid (FIG. 12F) (n=5-6 per group). FIGS. 12G and 12H are graphs showing that adoptive transfer of purified nuocytes from Il13^{+/+} (Rag2^{-/-}mice) into Il13^{-/-} recipients reconstitutes H3N1-induced AHR. Donor mice received IL-33 (1 μg, i.n.) prior to harvest of lung nuocytes. 5 days later, the lung nuocytes (Lin⁻ST2⁺ subsets) were sorted and adoptively transferred to Il13^{-/-} recipients (intra-tracheal injection, 10⁵ cells/mouse, n=4 per group) followed by infection with RSV. AIIR was measured (FIG. 12G), and cells in BAL fluid (FIG. 12H) were analyzed 6 days after the virus challenge. Data are representative of three independent experiments.

FIG. 13 shows the structures of certain c-Kit inhibitors.

### DETAILED DESCRIPTION OF THE INVENTION

The invention features methods, compositions, and kits that are useful for treating and preventing virus-induced respiratory pathology.

The invention is based, at least in part, on the discovery c-Kit inhibitors prevent and/or alleviate virus-induced airway hyperreactivity (AHR) and airway inflammation. It was previously shown that the development of virus-induced AHR required the presence of an innate lymphoid cell type called nuocytes (Chang, Y. J. et al., Nature Immunol. 12:631-638 (2011)) (also called natural helper cells, innate type 2 lymphoid cells or multipotent progenitor cells) (Moro, K. et al., Nature 463:540-544 (2010); Neill, D. R. et al., Nature 464:1367-1370 (2010); Saenz, S. A. et al., Nature 464:1371-1376 (2010); and Monticelli, L. A. et al., Nat. Immunol. 12:1045-1054 (2011)). Nuocytes are non-T, non-B cells that produce large quantities of IL-13 and IL-5, and express c-Kit (the receptor for stem cell factor) and receptors for IL-33 and IL-25. It was shown that viral infection induced the development of AHR independently of Th2 cells or adaptive immunity. It has now been surprisingly discovered that treatment of a viral infection (e.g., influenza and respiratory syncytial virus) with one to two doses of c-Kit kinase inhibitors blocks the function of nuocytes and halts the development of AHR and airway inflammation (Spits, H. et al., Ann. Rev. Immunol. 30:647-675 (2012); and Pearson, C. et al., Trends Immunol. 33:289-296 (2012)). In contrast, high doses of corticosteroids, a commonly used therapy for acute asthma, including asthma associated with viral infection, failed to prevent virus-induced AHR. These results indicate that targeting nuocytes with short courses of c-Kit kinase inhibitors may provide an effective therapy for asthma in patients infected with viruses that induce AHR and/or airway inflammation. Accordingly, the invention provides c-Kit kinase inhibitors that prevent and/or treat virus-induced respiratory pathology. The invention also relates to combination therapies including c-Kit kinase inhibitors.

c-Kit

c-Kit, also known as stem cell growth factor receptor or CD117, is a tyrosine-protein kinase that acts as a cell-surface receptor for stem cell factor. c-Kit is known to be involved in regulation of cell survival and proliferation; hematopoiesis; stem cell maintenance; gametogenesis; mast cell development, migration and function; and melanogenesis.

Full c-Kit nucleotide and polypeptide sequences, and variants related thereto, are well-known in the art. An exemplary nucleotide sequence can be found at NCBI Accession No. L04143:

```
        1 gagctcggat cccatcgcag ctaccgcgat gagaggcgct cgcggcgcct gggattttct
      61 ctgcgttctg ctcctactgc ttcgcgtcca gacaggtggg acaccgcggc tggctccccg
    121 accgtgcgac tactcgcgaa gcctgtgccc tgggagggtg gtaccgccat ggcatccgga
    181 gagaggactg cgggccctca gtgggcctgc gttccagcct ccggggagac tccaggtggc
    241 cctcggactc tccggcgccc tgcctcgctc acctgcgcga ggagacccca gctgctggtg
    301 gtgggggacg cgaatccggg gttcttcggg aatggggaca gcaagagggg gttaggcgtg
    361 agcgaggctg caggctccgt gcgagtttgg ggtggctttt gtgccgacgt tgcgcggggg
    421 cggaggcggg ggctcagggt ttgcaccgag cgccttctct ctcggtgcga ggccggccgc
    481 agcttccttt tgttaaaagt tgcgtgtgtg tgacggcgcc cgggctgcag cctcaacctc
    541 ctgggcttaa gtgatctccc acctcagcct cccgcctcag cctcccatgt agctgggaaa
    601 acaggttccc actaccatgc ccagctaatg ttttttctag tttttgtaga tgtgttgggc
    661 ggtgggggcg tctcactgcg ttgcccaggc tggtctcgaa ctcctaggct caagcaatct
    721 tcccacctca gcctcccaga gtgctgggat tacaggcgtg acgacggcac ctggccagca
    781 gtttgttctt taaaccctga aatgtatgtg aggaccatgt gtcacactag catggacgtt
    841 ttcccagcat ctagcatggt gctttgtaga tgataattaa tgaataggta tttgaataca
    901 tggaggcatg catggctgaa tgaagtggct gttgtaaaat ttctagggtt caggtttcat
    961 attcagagcc taaagtttgc atcttataaa ctaaatagtt tcctatctag gaaacctatt
  1021 taggcattag ggtgttaaaa caggtgtatc attttctgcc ttagtgttta gagatttgtg
  1081 aatagttctc cttttgatga acattgccat gtaaagagag ttatacagaa ataactgaat
  1141 tcacacagtt ttaaggaaaa gctattctat gtcatggtca tgtatattct gccaaagaga
  1201 gtgacaggcc agtagtttct tttttctttt tccccatagt gtgagatttt gtttctgttg
  1261 tttctaagct gggtgtctgc atgtccacac tgcgaagatg gcccatatca gaatgaaaac
  1321 ttgacccaat tgtatgttta gcccagagaa ggctggggca ttcagcacat cctggcttgg
  1381 ggaccaaatg tgaccctcag gattaattga gggttggaga aaataatttt atagatgaac
  1441 ttaagacatt ttagacagaa ctctcttttc agccataaat agcagggcag ctttgtccta
  1501 tttttattgt agagtacaca gaagatggaa ctcagtattg gaagaagtgc tttatttcgc
  1561 caaggaagaa gatcatactc aacacgattc tgtttttctt ggcaggctct tctcaaccat
  1621 ctgtgagtcc aggggaaccg tctccaccat ccatccatcc aggaaaatca gacttaatag
  1681 tccgcgtggg cgacgagatt aggctgttat gcactgatcc gggctttgtc aaatggactt
  1741 ttgagatcct ggatgaaacg aatgagaata agcagaatga atggatcacg gaaaaggcag
  1801 aagccaccaa caccggcaaa tacacgtgca ccaacaaaca cggcttaagc aattccattt
  1861 atgtgtttgt tagaggtaaa tgcttggctt tctgcagcag gtcatgtcac tttaggaggg
  1921 ttgcttttat gacaccgcag tttcatctat gaaatggcaa taatgatagt actgatcatg
  1981 ggaggggcaa tttgaagatt aaatgagatt aagtgtaatg gtccaagctt agtgcgtgat
  2041 acatggaaag cgtttaataa atgttaattc tcaatagtac tagatggata aattttgctt
  2101 ttgtttacac agaaaaaagc agccatttgg gccactagtc atgaaaggca acatattaga
  2161 tctttaaaag tgtttcagtg tctgtgacca gccattccaa ctactgattt ggatatgctt
  2221 cttatagatc ctgccaagct tttccttgtt gaccgctcct tgtatgggaa agaagacaac
  2281 gacacgctgg tccgctgtcc tctcacagac ccagaagtga ccaattattc cctcaagggg
  2341 tgccagggga agcctcttcc caaggacttg aggtttattc ctgaccccaa ggcgggcatc
  2401 atgatcaaaa gtgtgaaacg cgcctaccat cggctctgtc tgcattgttc tgtggaccag
  2461 gagggcaagt cagtgctgtc ggaaaaattc atcctgaaag tgaggccagg taccttgctt
  2521 tcttatctgc ctctggagtt gagaactcac ttatctaaag agacttctct tctcgttgat
  2581 cgtaagctgt acacatttga ggagaaatgg taaatcaaaa tttcatgcta taatacaaat
  2641 tatttgaggg gccacatttc ttttcattct agccttcaaa gctgtgcctg ttgtgtctgt
  2701 gtccaaagca agctatcttc ttagggaagg ggaagaattc acagtgacgt gcacaataaa
  2761 agatgtgtct agttctgtgt actcaacgtg gaaaagagaa aacagtcagg tgagtgaatc
  2821 gcttcattct tctcatgttc tgtctctgtg ggagatgata agttttctct ttcagaagag
  2881 tctgtcctga aactgcctcg actagtgcgt ctgtcagagg agaagttaat tgctgctatt
  2941 tttaatttat ctaggaaaga ttctgaatat aaattatatg gtaatcttca tttttttttc
  3001 tccttttctg aaaccagcag actaaactac aggagaaata taatagctgg catcacggtg
  3061 acttcaatta tgaacgtcag gcaacgttga ctatcagttc agcgagagtt aatgattctg
  3121 gagtgttcat gtgttatgcc aataatactt ttggatcagc aaatgtcaca acaaccttgg
  3181 aagtagtagg taaatacctc tatgggaatg tttaaattac tggcagtagt gaaagaagaa
  3241 attattagac agtttctttt ttatgtaaat ggaatgttga acagattctt agaattttgt
  3301 tatcactgaa tgaatgaaaa ttatccttgt agcctcttgc aatgaaagca caattctgtt
  3361 ttttttgtcc agtagttgta gataatgttt ctttctgtct tatttcattc taattagata
  3421 aaggattcat taatatcttc cccatgataa acactacagt atttgtaaac gatggagaaa
  3481 atgtagattt gattgttgaa tatgaagcat tccccaaacc tgaacaccag cagtggatct
  3541 ctctgaacag aaccttcact gataaatggg aagattatcc caagtctgag aatgaaagta
  3601 atatcaggta agaaatggac cttgccctgg ggattacaca ttaccccctt ttccagtggg
  3661 cttatcagat cttatttctg taacccgtaa atccacgaga agatacctgg taaagaagaa
  3721 agtctatttt gctaatactt tactgaatta aatgagttat atttttcctc aaacaggcat
  3781 agatttccag gtagaaactg aaaaagacat gccttccaag gcatgctatc cacaggtgat
  3841 tgactagttg tcttttcttt gtagatacgt aagtgaactt catctaacga gattaaaagg
  3901 caccgaagga ggcacttaca cattcctagt gtccaattct gacgtcaatg ctgccatagc
  3961 atttaatgtt tatgtgaata gtaagtaaca tgaagggctc ttttaatttt ttattctttt
  4021 aagttgtggc tcgtgtttgt aacagctgca aggactcaac ttgctgtact aaaggttgta
  4081 gggatttaga gagggagtga agtgaatgtt gctgaggttt tccagcactc tgacatatgg
  4141 ccatttctgt tttcctgtag caaaaccaga aatcctgact tacgacaggc tcgtgaatgg
  4201 catgctccaa tgtgtggcac caggattccc agagcccaca atagattggt atttttgtcc
  4261 aggaactgag cagaggtgag atgattattt ttggcactgc ttataatgca gaggggaagg
  4321 actgcaattc acttgaattt caaatatgtt ttctgatttt ttttaaaaaa gctttaactt
  4381 tgttttaaaa gtatgccaca tcccaagtgt tttatgtatt tatttatttt cctagagtaa
  4441 gccagggctt ttgttttctt ccctttagat gctctgcttc tgtactgcca gtggatgtgc
  4501 agacactaaa ctcatctggg ccaccgtttg gaaagctagt ggttcagagt tctatagatt
  4561 ctagtgcatt caagcacaat ggcacggttg aatgtaaggc ttacaacgat gtgggcaaga
  4621 cttctgccta ttttaacttt gcatttaaag gtaacaacaa aggtatattt ctttttaatc
  4681 caatttaagg ggatgtttag gctctgtcta ccatatcagt catgattttg agctcaatta
  4741 accctcacta aagggagtcg actcgatccc atcctgccaa agtttgtgat tccacatttc
  4801 tcttccattg tagagcaaat ccatccccac accctgttca ctcctttgct gattggtttc
  4861 gtaatcgtag ctggcatgat gtgcattatt gtgatgattc tgacctacaa atatttacag
  4921 gtaaccattt atttgttctc tctccagagt gctctaatga ctgagacaat aattattaaa
  4981 aggtgatcta tttttccctt tctccccaca gaaacccatg atgaagtaca gtggaaggtt
  5041 gttgaggaga taaatggaaa caattatgtt tacatagacc caacacaact tccttatgat
  5101 cacaaatggg agtttcccag aaacaggctg agttttggtc agtatgaaac aggggctttc
  5161 catgtcacct ttttgggtac acataacagt gactttaagg aactccagtg gcttcctttg
  5221 ttttgttcca cctgaaacaa tgagttttct gtgaaattgc gccccttttg ataggtttgc
  5281 catagagaac atcgtaggaa aatgtctctg gacaacattg tttttaattc ctttattgat
  5341 tttgaaactg cacaaatggt ccttcaattc caccaccagc accatcacca cttaccttgt
  5401 tgtcttcctt cctacaggga aaaccctggg tgctggagct ttcgggaagg ttgttgaggc
  5461 aactgcttat ggcttaatta agtcagatgc ggccatgact gtcgctgtaa agatgctcaa
  5521 gcgtaagttc ctgtatggta ctgcatgcgc ttgacatcag tttgccagtt gtgctttttg
  5581 ctaaaatgca tgtttccaat tttagcgagt gcccatttga cagaacggga agccctcatg
  5641 tctgaactca aagtcctgag ttaccttggt aatcacatga atattgtgaa tctacttgga
  5701 gcctgcacca ttggaggtaa agccgtgtcc aagctgcctt ttattgtctg tcaggttatc
  5761 aaaacatgac attttaatat gattttggca atgctagatt ataaactgct tggaagattt
  5821 ttttacccag actgttgttc tctcttgcta gattttgttt tcctcattgt tcttaagaat
  5881 atatgggatt gtattgggac taagtagtct gatccactga agctgaatat taatggccat
  5941 gaccaccctt gggtattttt atgggaggca gaattaatct atatatctca ccttctttct
  6001 aaccttttct tatgtgcttt tagggcccac cctggtcatt acagaatatt gttgctatgg
  6061 tgatcttttg aattttttga gaagaaaacg tgattcattt atttgttcaa agcaggaaga
  6121 tcatgcagaa gctgcacttt ataagaatct tctgcattca aaggagtctt cctggtaaga
  6181 ctgatttaca taaatagtta gctgttgaca ggcagttcat ggggaactct ttattcaaac
  6241 tttacatgac tttcctcaaa ttggtccagt ctattatgta gcaaagggga tgaggaggta
  6301 gagcatgacc catgagtgcc cttctacatg tcccacttga ttcagtcatg acttgtttca
  6361 tctctcccag cagcgatagt actaatgagt acatggacat gaaacctgga gtttcttatg
  6421 ttgtcccaac caaggccgac aaaaggagat ctgtgagaat aggtgagtac ctacctatca
  6481 agcaaccaag agtaacttta cagagagtat gtatatcatg ctaatgtgga atataacatc
  6541 attccagtag caatgatgca gaccagttct gctttatggt agcagtgcca atggtcaatg
  6601 gcagttaggg gcaagttcac attagttcat tcattaccag cctttggtat gtcattgcca
  6661 ctgtcttttc ctttcctgac ctttatggtt gtaattgcta agaaaaatcc tctcttcctc
  6721 acaggctcat acatagaaag agatgtgact cccgccatca tggaggatga cgagttggcc
  6781 ctagacttag aagacttgct gagcttttct taccaggtgg caaagggcat ggctttcctc
  6841 gcctccaaga atgtaagtgg gagtgattct ctaaagagtt ttgtgttttg tttttttgat
  6901 tttttttttt tttttttttt ttttgagaac agagcatttt agagccatag ttaaaagcag
  6961 aatgtcattt aaaacaaaag tattggattt tttataatat aagcaacact atagtattaa
  7021 aaagttagtt ttcactcttt acaagttaaa atgaatttaa atggttttct tttctcctcc
  7081 aacctaatag tgtattcaca gagacttggc agccagaaat atcctcctta ctcatggtcg
  7141 gatcacaaag atttgtgatt ttggtctagc cagagacatc aagaatgatt ctaattatgt
  7201 ggttaaagga aacgtgagta cccattctct gcttgacagt cctgcaaagg atttttagtt
  7261 tcaactttcg ataaaaattg tttccgtgac tttcataatg taaatcctgt ctagggatat
  7321 cacacatttt agcagtcaaa tgtatttcag aggtgattgg gatcatctga gttcatatag
  7381 gtaaaaggtt tttgtgagat ggtactcaag ttatcactcc acatttcagc aacagcagca
  7441 tctataagaa tatcttctgt tcaattttgt tgagcttctg aattaacatt attgactctg
  7501 ttgtgcttct attacaggct cgactacctg tgaagtggat ggcacctgaa agcattttca
  7561 acgtgtatac acgtttgaaa gtgacgtctg gtcctatcgg atttttcttt gggagctgtt
  7621 ctctttaggt aaaatgatcc ttgccaaaag acaacttcat tagactcaga gcatcttgaa
  7681 gtttcattgg tgtcctgctt ccttgtgatt aacactgctt tgcaaactgt gtctcaggaa
  7741 gcagccccta tcctggaatg ccggtcgatt ctaagttcta caagatgatc aaggaaggct
  7801 tccggatgct cagcctgaac acgcacctgc tgaaatgtaa gagccaaaaa atttttcctt
  7861 taggtcacgt tttccctttt atttttcttt ttagagacag aaacccagat gttgagggtt
  7921 ttcataacac agtttgaaat gtcacttgga ttctttatga cacactggtc aaatgtcatt
  7981 tctgtagttt attttcataa tctcttgtca ccaaaaatac agaaagtttc agtaatattt
  8041 catacatgca gtgttttatg ttatctatat gtcagtccat atgtccagtt gcatagccct
  8101 ggaattatta ctgaagttgc tggatgccca tacatttgaa aacaagctga gggcattgag
  8161 gagggatagt aaatggccct tgtcttgcag gtatgacata atgaagactt gctgggatgc
  8221 agatccccta aaaagaccaa cattcaagca aattgttcag ctaattgaga agcagatttc
  8281 agagagcacc aatcatgtga gtataccctg gccaggcata gaatccccct tctcccagtt
  8341 ccaggtgtgt cctcctcctc aggctttcag ggtgaggact aacctcccaa ccccttctct
  8401 cctaatctta ggttgcaaat tgggcttcag gtaggggaag taaagcaatg gaaactagtt
  8461 cttttaagag ttccatcagt tagttgtgat cttgacactg taagtatgcc ttttgttgct
  8521 atgttcgttg tagggactgc tgtattgact atgggcttgt tttctccaga tttactccaa
  8581 cttagcaaac tgcagcccca accgacagaa gcccgtggta gaccattctg tgcggatcaa
  8641 ttctgtcggc agcaccgctt cctcctccca gcctctgctt gtgcacgacg atgtctgagc
  8701 agaatcagtg tttgggtcac ccctccagga atgatctctt cttttggctt ccatgatggt
  8761 tattttcttt tctttcaact tgcatccaac tccaggatag tgggcacccc actgcaatcc
  8821 tgtctttctg agcacacttt agtggccgat gatttttgtc atcagccacc atcctattgc
  8881 aaaggttcca actgtatata ttcccaatag caacgtagct tctaccatga acagaaaaca
  8941 ttctgatttg gaaaaagaga gggaggtatg gactgggggc cagagtcctt tccaaggctt
  9001 ctccaattct gcccaaaaat atggttgata gtttacctga ataaatggta gtaatcacag
  9061 ttggccttca gaaccatcca tagtagtatg atgatacaag attagaagct gaaaacctaa
  9121 gtcctttatg tggaaaacag aacatcatta gaacaaagga cagagtatga acacctgggc
  9181 ttaagaaatc tagtatttca tgctgggaat gagacatagg ccatgaaaaa aatgatcccc
  9241 aagtgtgaac aaaagatgct cttctgtgga ccactgcatg agcttttata ctaccgacct
  9301 ggtttttaaa tagagtttgc tattagagca ttgaattgga gagaaggcct ccctagccag
  9361 cacttgtata tacgcatcta taaattgtcc gtgttcatac atttgagggg aaaacaccat
  9421 aaggtttcgt ttctgtatac aaccctggca ttatgtccac tgtgtataga agtagattaa
  9481 gagccatata agtttgaagg aaacagttaa taccattttt taaggaaaca atataaccac
  9541 aaagcacagt ttgaacaaaa tctcctcttt tagctgatga acttattctg tagattctgt
  9601 ggaacaagcc tatcagcttc agaatggcat tgtactcaat ggatttgatg ctgtttgaca
  9661 aagttactga ttcactgcat ggctcccaca ggagtgggaa aacactgcca tcttagtttg
  9721 gattcttatg tagcaggaaa taaagtatag gtttagcctc cttcgcaggc atgtcctgga
  9781 caccgggcca gtatctatat atgtgtatgt acgtttgtat gtgtgtagac aaatatttgg
  9841 aggggtattt ttgccctgag tccaagaggg tcctttagta cctgaaaagt aacttggctt
  9901 tcattattag tactgctctt gtttcttttc acatagctgt ctagagtagc ttaccagaag
  9961 cttccatagt ggtgcagagg aagtggaagg catcagtccc tatgtatttg cagttcacct
10021 gcacttaagg cactctgtta tttagactca tcttactgta cctgttcctt agaccttcca
10081 taatgctact gtctcactga aacatttaaa ttttaccctt tagactgtag cctggatatt
10141 attcttgtag tttacctctt taaaaacaaa acaaaacaaa acaaaaaact ccccttcctc
10201 actgcccaat ataaaaggca aatgtgtaca tggcagagtt tgtgtgttgt cttgaaagat
10261 tcaggtatgt tgcctttatg gtttccccct tctacatttc ttagactaca tttagagaac
10321 tgtggccgtt atctggaagt aaccatttgc actggagttc tatgctctcg cacctttcca
10381 aagttaacag attttggggt tgtgttgtca cccaagagat tgttgtttgc catactttgt
10441 ctgaaaaatt cctttgtgtt tctattgact tcaatgatag taagaaaagt ggttgttagt
10501 tatagatgtc taggtacttc aggggcactt cattgagagt tttgtcttgc catactttgt
10561 ctgaaaaatt cctttgtgtt tctattgact tcaatgatag taagaaaagt ggttgttagt
10621 tatagatgtc taggtacttc aggggcactt cattgagagt tttgtcttgg atattcttga
10681 aagtttatat ttttataatt ttttcttaca tcagatgttt ctttgcagtg gcttaatgtt
10741 tgaaattatt ttgtggcttt ttttgtaaat attgaaatgt agcaataatg tcttttgaat
10801 attcccaagc ccatgagtcc ttgaaaatat tttttatata tacagtaact ttatgtgtaa
10861 atacataagc ggcgtaagtt taaaggatgt tggtgttcca cgtgttttat tcctgtatgt
10921 tgtccaattg ttgacagttc tgaagaattc taataaaatg tacatatata aatcaa
```

An exemplary polypeptide sequence can be found at NCBI Accession No. NP_{—}000213:

```
    1 mrgargawdf lcvlllllrv qtgssqpsvs pgepsppsih pgksdlivrv gdeirllctd
  61 pgfvkwtfei ldetnenkqn ewitekaeat ntgkytctnk hglsnsiyvf vrdpaklflv
121 drslygkedn dtlvrcpltd pevtnyslkg cqgkplpkdl rfipdpkagi miksvkrayh
181 rlclhcsvdq egksvlsekf ilkvrpafka vpvvsvskas yllregeeft vtctikdvss
241 svystwkren sqtklqekyn swhhgdfnye rqatltissa rvndsgvfmc yanntfgsan
301 vtttlevvdk gfinifpmin ttvfvndgen vdliveyeaf pkpehqqwiy mnrtftdkwe
361 dypksenesn iryvselhlt rlkgteggty tflvsnsdvn aaiafnvyvn tkpeiltydr
421 lvngmlqcva agfpeptidw yfcpgtegrc sasvlpvdvq tlnssgppfg klvvqssids
481 safkhngtve ckayndvgkt sayfnfafkg nnkeqihpht lftplligfv ivagmmciiv
541 miltykylqk pmyevqwkvv eeingnnyvy idptqlpydh kwefprnrls fgktlgagaf
601 gkvveatayg liksdaamtv avkmlkpsah lterealmse lkvlsylgnh mnivnllgac
661 tiggptivit eyccygdlln flrrkrdsfi cskqedhaea alyknllhsk esscsdstne
721 ymdmkpgvsy vvptkadkrr svrigsyier dvtpaimedd elaldledll sfsyqvakgm
781 aflaskncih rdlaarnill thgritkicd fglardiknd snyvvkgnar lpvkwmapes
841 ifncvytfes dvwsygiflw elfslgsspy pgmpvdskfy kmikegfrml spehapaemy
901 dimktcwdad plkrptfkqi vgliekgise stnhiysnla ncspnrqkpv vdhsvrinsv
961 gstasssqpl lvhddv
```

As described in detail below, it has now been discovered that c-Kit is also involved in virus-induced respiratory disease and pathology (e.g., virus-induced acute asthma and its associated pathologies, e.g., airway inflammation and airway hyperreactivity) Inhibition of c-Kit reduces and/or prevents virus-induced airway hyperreactivity and inflammation. Accordingly, the present invention provides methods that are useful for treating and preventing virus-induced respiratory pathologies.

c-Kit Kinase Inhibitors

In general, the invention features c-Kit kinase inhibitors that inhibit or prevent virus-induced respiratory pathology. The c-Kit kinase inhibitors can be any agent (e.g., a small molecule chemical compound, antibody, nucleic acid molecule, or polypeptide) that reduces expression and/or biological activity c-Kit.

c-Kit kinase inhibitors, as well as methods for making such inhibitors, are well-known in the art. Exemplary inhibitors include, but are not limited to, those c-Kit inhibitors described in U.S. Patent Application Publication Nos. 20120053186; 20110312992; 20110293615; 20110281813; 20110230482; 20110223165; 20110201620; 20110183997; 20110166174; 20100234406; 20100190811; 20100184791; 20100143935; 20100081656; 20090281115; 20090264649; 20090170862; 20090136517; 20090105297; 20090076046; 20090062275; 20090053236; 20090012094; 20080274469; 20080255141; 20080221187; 20080221153; 20080176846; 20080167308; 20080146585; 20080139597; 20080139559; 20080096892; 20080004279; 20070253951; 20070232669; 20070225293; 20070149538; 20070032521; 20070032519; 20060189629; 20060166281; 20060116402; 20060058340; 20060058339; 20060035951; 20060035921; 20050288353; 20050239852; 20050192314; 20050165074; 20050130153; 20050054617; 20040266797; 20040266779; 20040253205; 20040242601; 20040241226; 20040002534; and 20020010203; and U.S. Pat. Nos. 8,106,068; 7,994,159; 7,947,708; 7,915,391; 7,893,075; 7,846,941; 7,767,673; 7,727,731; 7,678,792; 7,638,523; 7,592,349; 7,563,894; 7,521,448; 7,514,447; 7,498,342; 7,485,658; 7,442,709; 7,419,995; 7,303,893; 7,285,413; 7,211,600; 5,821,108; and 5,989,849, each of which is hereby incorporated by reference in its entirety.

Suitable c-Kit inhibitors include analogs and derivatives thereof. The analogs and derivatives do not destroy the biological activity of the c-Kit inhibitor, and preferably confers upon the c-Kit inhibitor advantageous properties in vivo, such as uptake, duration of action, or onset of action. Another example is a prodrug of a c-Kit inhibitor. The prodrug is itself biologically inactive, but is converted in vivo to the biologically active form of the c-Kit inhibitor.

c-Kit inhibitor derivatives include pharmaceutically acceptable salts of the c-Kit inhibitor. The c-Kit inhibitor derivative has equivalent or near equivalent physiological functionality to the c-Kit inhibitor. The derivative may confer upon the c-Kit inhibitor advantageous properties such as improved storage stability or enhanced solubility.

In another aspect, the c-Kit inhibitor is a compound represented by the following structure (I):

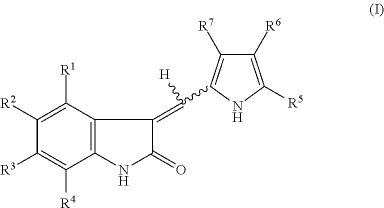

wherein:
R₁ is selected from the group consisting of hydrogen, halo, alkyl, cyclkoalkyl, aryl, heteroaryl, heteroalicyclic, hydroxy, alkoxy, —(CO)R₁₅, —NR₁₃R₁₄, —(CH₂)ᵣR₁₆ and —C(O)NR₈R₉;
R₂ is selected from the group consisting of hydrogen, halo, alkyl, trihalomethyl, hydroxy, alkoxy, cyano, —NR₁₃R₁₄, —NR₁₃C(O)R₁₄, —C(O)R₁₅, aryl, heteroaryl, —S(O)₂NR₁₃R₁₄ and —SO₂R₂₀ (wherein R₂₀ is alkyl, aryl, aralkyl, heteroaryl and heteroaralkyl);
R₃ is selected from the group consisting of hydrogen, halogen, alkyl, trihalomethyl, hydroxy, alkoxy, —(CO)R₁₅, —NR₁₃R₁₄, aryl, heteroaryl, —NR₁₃S(O)₂R₁₄, —S(O)₂R₁₃R₁₄, —NR₁₃C(O)R₁₄, —NR₁₃C(O)OR₁₄ and —SO₂R₂₀ (wherein R₂₀ is alkyl, aryl, aralkyl, heteroaryl and heteroaralkyl);
R₄ is selected from the group consisting of hydrogen, halogen, alkyl, hydroxy, alkoxy and —NR₁₃R₁₄;
R₅ is selected from the group consisting of hydrogen, alkyl and —C(O)R₁₀;
R₆ is selected from the group consisting of hydrogen, alkyl and —C(O)R₁₀;
R₇ is selected from the group consisting of hydrogen, alkyl, aryl, heteroaryl, —C(O)R₁₇ and —C(O)R₁₀; or
R₆ and R₇ may combine to form a group selected from the group consisting of —(CH₂)₄—, —(CH₂)₅₋and —(CH₂)₆₋; with the proviso that at least one of R₅, R₆ or R₇ must be —C(O)R₁₀;
R₈ and R₉ are independently selected from the group consisting of hydrogen, alkyl and aryl;
R₁₀ is selected from the group consisting of hydroxy, alkoxy, aryloxy, —N(R₁₁) (CH₂)ₙR₁₂, and —NR₁₃R₁₄;
R₁₁ is selected from the group consisting of hydrogen and alkyl;
R₁₂ is selected from the group consisting of —NR₁₃R₁₄, hydroxy, —C(O)R₁₅, aryl, heteroaryl, —N⁺(O)R₁₃ R₁₄,—N(OH)R₁₃and —NHC(O)Rₐ (wherein Rₐ is unsubstituted alkyl, haloalkyl, or aralkyl);
R₁₃ and R₁₄ are independently selected from the group consisting of hydrogen, alkyl, cyanoalkyl, cycloalkyl, aryl and heteroaryl; or
R₁₃ and R₁₄ may combine to form a heterocyclo group;
R₁₅ is selected from the group consisting of hydrogen, hydroxy, alkoxy and aryloxy;
R₁₆ is selected from the group consisting of hydroxy, —C(O)R₁₅, —NR₁₃R₁₄ and —C(O)NR₁₃R₁₄;
R₁₇ is selected from the group consisting of alkyl, cycloalkyl, aryl and heteroaryl;
R₂₀ is alkyl, aryl, aralkyl or heteroaryl; and
n and r are independently 1, 2, 3, or 4;
or a pharmaceutically acceptable salt thereof.

In another aspect, the c-Kit inhibitor is a compound represented by the following structure (II):

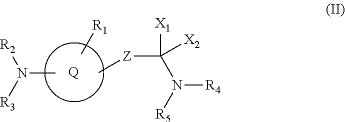

wherein:
Q is:
   (1) a 5-membered heteroaryl ring;
   (2) a 6-membered heteroaryl ring; or (3) an aryl ring; optionally substituted with one or more groups R₁;
   Z is:
      (1) a single bond;
      (2) —R₁₅C═CH—; or
      (3) —(CH₂)_{m -}, where m is 1 to 2;
   X₂ and X₂ are each hydrogen, or together form ═O or ═S;
   R₁ is:
      (1) hydrogen or R₆, where R₆ is alkyl, alkenyl, alkynyl, cycloalkyl, cycloalkylalkyl, cycloalkenyl, cycloalkenylalkyl, aryl, aralkyl, heterocyclo, or heterocycloalkyl, each of which is unsubstituted or substituted with Z₁, Z₂ and one or more (preferably, one or two) groups Z₃;
      (2) —OH or —OR₆;
      (3) —SH or —SR₆;
      (4) —C(O)₂H, —C(O)_{q}R₆, or —o—C(O)_{q}R₆, where q is 1 or 2;
      (5) —SO₃H or —S(O)_{q}R₆;
      (6) halo;
      (7) cyano;
      (8) nitro;
      (9) —Z₄—NR₇R₈;
      (10) —Z₄—N(R₉)—Z₅—NR₁₀R₁₁;
      (11) —Z₄^{—N(R}₁₂)—Z₅—R₆;
      (12) —P(O)(OR₆)₂;
   R₂ and R₃ are each independently:
      (1) hydrogen or R₆;
      (2) —Z₄—R₆; or
      (3) —Z₁₃—NR₇R₈;
   R₄ and R₅:
      (1) are each independently hydrogen or R₆;
      (2) —Z₄—N(R₉)—Z₅—NR₁₀R₁₁;
      (3) —N(R₉)Z₄R₆; or
      (4) together with the nitrogen atom to which they are attached complete a 3-to 8-membered saturated or unsaturated heterocyclic ring which is unsubstituted or substituted with Z₂, Z₂ and Z₃, which heterocyclic ring may optionally have fused to it a benzene ring itself unsubstituted or substituted with Z₁, Z₂ and Z₃ ;
   R₇, R₈, R₉, R₁₀, R₁₁, and R₁₂;
      (1) are each independently hydrogen or R₆;
      (2) R₇ and R₈ may together be alkylene, alkenylene or heteroalkyl, completing a 3-to 8-membered saturated or unsaturated ring with the nitrogen atom to which they are attached, which ring is unsubstituted or substituted with Z₁, Z₂ and Z₃; or
      (3) any two of R₉, R₁₀, and R₁₁ may together be alkylene or alkenylene completing a 3-to 8-membered saturated or unsaturated ring together with the nitrogen atoms to which they are attached, which ring is unsubstituted or substituted with Z₁, Z₂ and Z₃;
   R₁₃ is:
      (1) cyano;
      (2) nitro;
      (3) —NH₂;
      (4) —NHOalkyl;
      (5) —OH;
      (6) —NHOaryl;
      (7) —NHCOOalkyl;
      (8) —NHCOOaryl;
      (9) —NHSO₂alkyl;
      (10) —NHSO₂aryl;
      (11) aryl;
      (12) heteroaryl;
      (13) —Oalkyl; or
      (14) —Oaryl;
   R₁₄ is:
      (1) —NO₂;
      (2) —COOalkyl; or
      (3) —COOaryl;
   R₁₅ is:
      (1) hydrogen;
      (2) alkyl;
      (3) aryl;
      (4) arylalkyl; or
      (5) cycloalkyl;
   Z₁, Z₂ and Z₃ are each independently:
      (1) hydrogen or Z₆, where Z₆ is (i) alkyl, alkenyl, alkynyl, cycloalkyl, cycloalkylalkyl, cycloalkenyl, cycloalkenylalkyl, aryl, aralkyl, alkylaryl, cycloalkylaryl, heterocyclo, or heterocycloalkyl; (ii) a group (i) which is itself substituted by one or more of the same or different groups (i); or (iii) a group (i) or (ii) which is substituted by one or more of the following groups (2) to (16) of the definition of Z₁, Z₂ and Z₃;
      (2) —OH or —OZ₆;
      (3) —SH or —SZ₆;
      (4) —C(O)_{q}H, —C(O)_{q}Z₆, or —O—C(O)_{q}Z₆;
      (5) —SO₃H, —S(O)_{q}Z₆; or S(O)_{q}N(Z₉)Z₆;
      (6) halo;
      (7) cyano;
      (8) nitro;
      (9) —Z₄—NZ₇Z₈;
      (10) —Z₄—N(Z₉)—Z₅—NZ₇Z₈;
      (11) —Z₄—N(Z₁₀)—Z₅—Z₆;
      (12) —Z₄—N(Z₁₀)—Z₅—H;
      (13) oxo;
      (14) —O—C(O)—Z₆;
      (15) any two of Z₁, Z₂, and Z₃ may together be alkylene or alkenylene completing a 3-to 8-membered saturated or unsaturated ring together with the atoms to which they are attached; or
      (16) any two of Z₁, Z₂, and Z₃ may together be —O—(CH₂)ᵣ—O—, where r is 1 to 5, completing a 4-to 8-membered saturated or unsaturated ring together with the atoms to which they are attached;
   Z₄ and Z₅ are each independently:
      (1) a single bond;
      (2) —Z₁₁—S(O)_{q}—Z₁₂—;
      (3) —Z₁₁—C(O)—Z₁₂—;
      (4) —Z₁₁—C(S)—Z₁₂—;
      (5) —Z₁₁—O—Z₁₂—;
      (6) —Z₁₁—S—Z₁₂—;
      (7) —Z₁₁—O—C(O)—Z₁₂—; or
      (8) —Z₁₁—C(O)—O—Z₁₂—;
   Z₇, Z₈, Z₉ and Z₁₀:
      (1) are each independently hydrogen or Z₆;
      (2) Z₇ and Z₈, or Z₆ and Z₁₀, may together be alkylene or alkenylene, completing a 3-to 8-membered saturated or unsaturated ring together with the atoms to which they are attached, which ring is unsubstituted or substituted with Z₁, Z₂ and Z₃; or
      (3) Z₇ or Z₈, together with Z₉, may be alkylene or alkenylene completing a 3-to 8-membered saturated or unsaturated ring together with the nitrogen atoms to which they are attached, which ring is unsubstituted or substituted with Z₁, Z₂ and Z₃;
   Z₁₁ and Z₁₂ are each independently:
      (1) a single bond;
      (2) alkylene;
      (3) alkenylene; or
      (4) alkynylene; and
   Z₁₃ is:
      (1) a single bond;
      (2) —Z₁₁—S(O)_{q}—Z₁₂—;
      (3) —Z₁₁—C(O)—Z₁₂—;
      (4) —Z₁₁—C(S)—Z₁₂—;
      (5) —Z₁₁—O—Z₁₂—;
      (6) —Z₁₁—S—Z₁₂—;
      (7) —Z₁₁—O—C(O)—Z₁₂—;
      (8) —Z₁₁—C(O)—O—Z₁₂—;
      (9) —C(NR₁₃)—;
      (10) —C(CHR₁₄)—; or
      (11) —C(C(R₁₄)₂)—;
         or a pharmaceutically acceptable salt thereof.

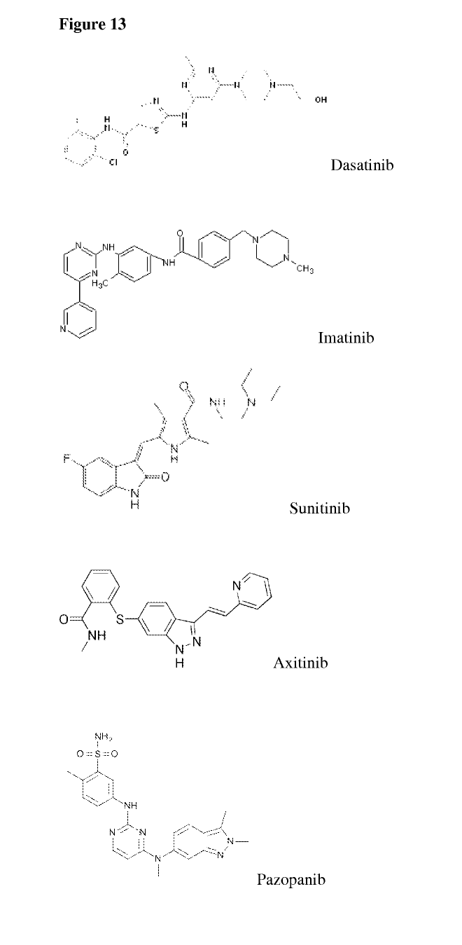

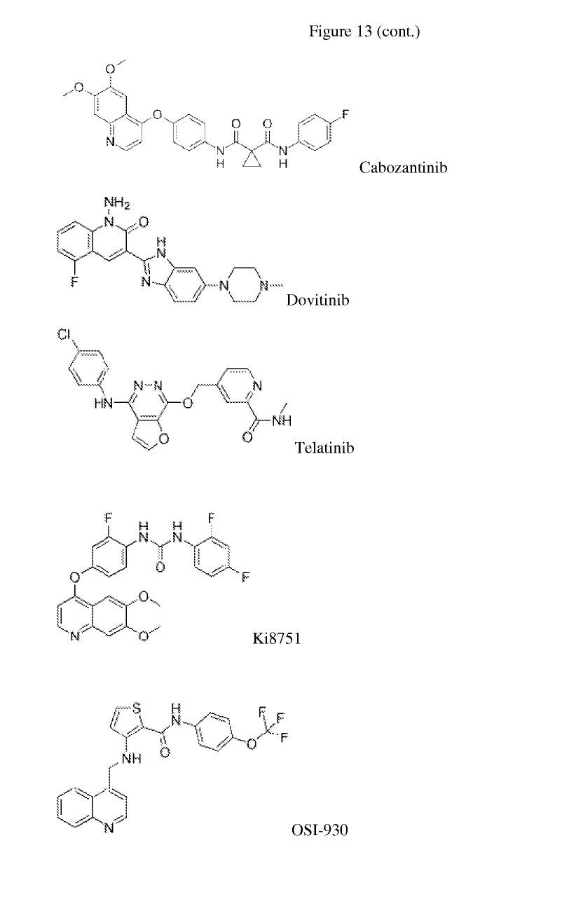

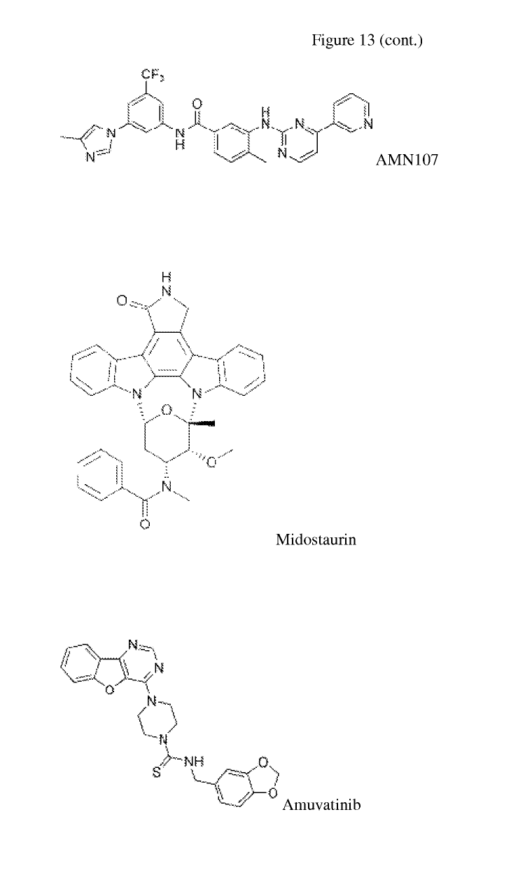

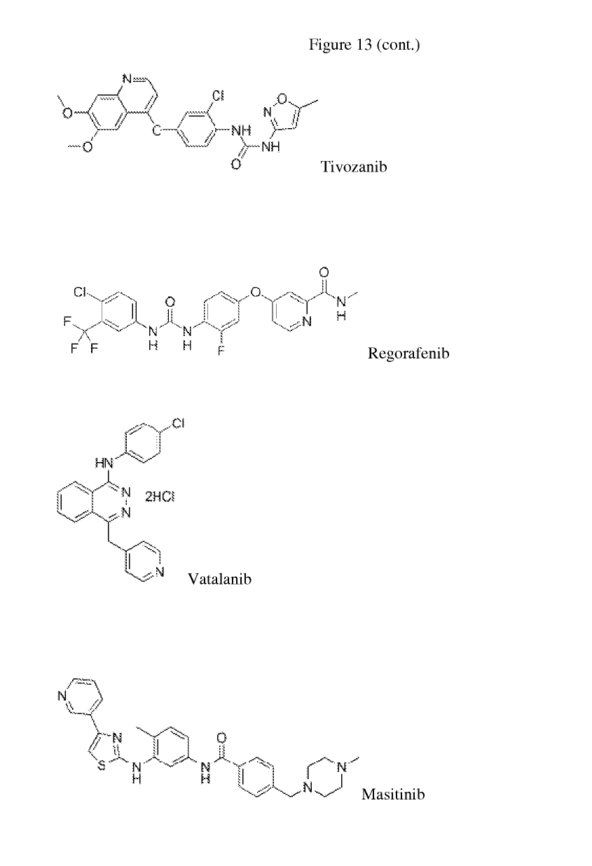

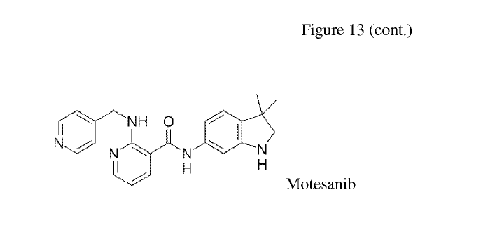

In aspects of the invention, the c-Kit inhibitor is dasatinib; imatinib; sunitinib; axitinib; pazopanib; cabozantinib; dovitinib; telatinib; Ki8751; OSI-930; AMN107; midostaurin; amuvatinib; tivozanib; regorafenib; vatalanib; masitinib; motesanib; or a salt, analog, or derivative thereof. The structures of these compounds are shown in FIG. 13. In embodiments, the c-Kit kinase inhibitor is imatinib; masitinib; or a salt, analog, or derivative thereof.

In another aspect of the invention, the agent is a nucleic acid molecule that reduces the expression and/or biological activity of c-Kit. Such oligonucleotides are well-known in the art and include single and double stranded nucleic acid molecules (e.g., DNA, RNA, and analogs thereof) that bind a nucleic acid molecule that encodes c-Kit (e.g., antisense molecules, siRNA, shRNA) as well as nucleic acid molecules that bind directly to c-Kit to modulate its biological activity (e.g., aptamers). See, e.g., U.S. Pat. No. 5,989,849, which is hereby incorporated by reference.

In yet another aspect of the invention, the agent is an antibody or antibody fragment that specifically binds to a molecule (e.g., c-Kit or a molecule that interacts with c-Kit) and reduces the expression and/or biological activity of c-Kit (e.g., binding of stem cell factor to activate nuocytes). Such antibodies and antibody fragments are well-known in the art. See, e.g., U.S. Pat. Nos. 7,915,391 and 5,545,533; and U.S. Patent Application Publication Nos. 20110223165 and 20070253951, each of which is hereby incorporated by reference. As described in detail below, methods for making and screening such antibodies and antibody fragments are also within the purview of the skilled artisan.

Ribozymes

Catalytic RNA molecules or ribozymes that include an antisense c-Kit sequence of the present invention can be used to inhibit expression of a c-Kit nucleic acid molecule in vivo. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591 (1988); Antisense and Ribozyme Methodology (Ian Gibson ed., 2008); Ribozyme Protocols (Philip C. Turner ed., 1997); Ribozymes and siRNA Protocols (Mouldy Sioud ed., 2004); siRNA and miRNA Gene Silencing (Mouldy Sioud ed., 2009); and Therapeutic Applications of Ribozymes (Kevin J. Scanlon ed., 1998), each of which is hereby incorporated by reference.

Accordingly, the invention also features a catalytic RNA molecule that includes, in the binding arm, an antisense RNA having between eight and nineteen consecutive nucleobases. In embodiments, the catalytic nucleic acid molecule is formed in a hammerhead or hairpin motif. Examples of such hammerhead motifs or hairpin motifs are described by Shelburne et al., Clin. Immunol. 93:46-58 (1999). These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Small hairpin RNAs consist of a stem-loop structure with optional 3′ UU-overhangs. While there may be variation, stems can range from 21 to 31 by (desirably 25 to 29 bp), and the loops can range from 4 to 30 by (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

siRNA

Short twenty-one to twenty-five nucleotide double-stranded RNAs are effective at down-regulating gene expression. (Zamore et al., Cell 101:25-33 (2000); and Elbashir et al., Nature 411:494-498 (2001)). The therapeutic effectiveness of an siRNA approach in mammals was demonstrated in vivo by McCaffrey et al. Nature 418:38-39 (2002).

Given the sequence of a target gene, siRNAs may be designed to inactivate that gene. Such siRNAs, for example, could be administered directly to an affected tissue, or administered systemically. The nucleic acid sequence of a c-Kit gene can be used to design small interfering RNAs (siRNAs). The 21 to 25 nucleotide siRNAs may be used, for example, as therapeutics to treat a nerve injury.

The inhibitory nucleic acid molecules of the present invention may be employed as double-stranded RNAs for RNA interference (RNAi)-mediated knock-down of c-Kit expression. In embodiments, c-Kit expression is reduced in a nuocyte. RNAi is a method for decreasing the cellular expression of specific proteins of interest (reviewed in Tuschl, Chembiochem. 2:239-245 (2001); Sharp, Genes & Devel. 15:485-490 (2000); Hutvagner and Zamore, Curr. Opin. Genet. Devel. 12:225-232 (2002); Hannon, Nature 418:244-251 (2002); Ribozymes and siRNA Protocols (Mouldy Sioud ed., 2004); and siRNA and miRNA Gene Silencing (Mouldy Sioud ed., 2009)). The introduction of siRNAs into cells either by transfection of dsRNAs or through expression of siRNAs using a plasmid-based expression system is increasingly being used to create loss-of-function phenotypes in mammalian cells.

In embodiments, double-stranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al., Science 296:550-553 (2002); Paddison et al., Genes & Devel. 16:948-958 (2002); Paul et al., Nature Biotechnol. 20:505-508 (2002); Sui et al., Proc. Natl. Acad. Sci. USA 99:5515-5520 (2002); Yu et al., Proc. Natl. Acad. Sci. USA 99:6047-6052 (2002); Miyagishi et al., Nature Biotechnol. 20:497-500 (2002); and Lee et al., Nature Biotechnol. 20:500-505 (2002), which are hereby incorporated by reference.

Small hairpin RNAs (shRNAs) comprise an RNA sequence having a stem-loop structure. A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The term “hairpin” is also used herein to refer to stem-loop structures. Such structures are well-known in the art and the term is used consistently with its known meaning in the art. As is known in the art, the secondary structure does not require exact base-pairing. Thus, the stem can include one or more base mismatches or bulges. Alternatively, the base-pairing can be exact, i.e., not include any mismatches. The multiple stem-loop structures can be linked to one another through a linker, such as, for example, a nucleic acid linker, a miRNA flanking sequence, other molecule, or some combination thereof.

As used herein, the term “small hairpin RNA” includes a conventional stem-loop shRNA, which forms a precursor miRNA (pre-miRNA). While there may be some variation in range, a conventional stem-loop shRNA can comprise a stem ranging from 19 to 29 bp, and a loop ranging from 4 to 30 bp. “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. In some instances the precursor miRNA molecule can include more than one stem-loop structure. MicroRNAs are endogenously encoded RNA molecules that are about 22-nucleotides long and generally expressed in a highly tissue-or developmental-stage-specific fashion and that post-transcriptionally regulate target genes. More than 200 distinct miRNAs have been identified in plants and animals. These small regulatory RNAs are believed to serve important biological functions by two prevailing modes of action: (1) by repressing the translation of target mRNAs, and (2) through RNA interference (RNAi), that is, cleavage and degradation of mRNAs. In the latter case, miRNAs function analogously to small interfering RNAs (siRNAs). Thus, one can design and express artificial miRNAs based on the features of existing miRNA genes.

In this regard, short hairpin RNAs can be designed to mimic endogenous miRNAs. Many miRNA intermediates can be used as models for shRNA or shRNAmir, including without limitation an miRNA comprising a backbone design of miR-15a, -16, -19b, -20, -23a, -27b, -29a, -30b, -30c, -104, -132s, -181, -191, -223 (see U.S. Publication No. 2005/0075492). In embodiments, shRNA molecules are designed based on the human miR-30 sequence, redesigned to allow expression of artificial shRNAs by substituting the stem sequences of the pri-miR-30 with unrelated base-paired sequences (see Siolas et al., Nat. Biotech. 23:227-231 (2005); Silva et al., Nat. Genet. 37:1281-1288 (2005); Zeng et al., Molec. Cell 9:1327-1333 (2002)). The natural stem sequence of the miR-30 can be replaced with a stem sequence from about 16 to about 29 nucleotides in length, in particular from about 19 to 29 nucleotides in length. The loop sequence can be altered such that the length is from about 4 to about 23 nucleotides. In embodiments, the stem of the shRNA molecule is about 22 nucleotides in length. In other embodiments, the stem is about 29 nucleotides in length. Thus, the invention can be practiced using shRNAs that are synthetically produced, as well as microRNA (miRNA) molecules that are found in nature and can be remodeled to function as synthetic silencing short hairpin RNAs.

shRNAs can be expressed from DNA vectors to provide sustained silencing and high yield delivery into almost any cell type. In embodiments, the vector is a viral vector. Exemplary viral vectors include retroviral, including lentiviral, adenoviral, baculoviral and avian viral vectors, and including such vectors allowing for stable, single-copy genomic integrations. Retroviruses from which the retroviral plasmid vectors can be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus. A retroviral plasmid vector can be employed to transduce packaging cell lines to form producer cell lines. Examples of packaging cells that can be transfected include, but are not limited to, the PE501, PA317, R-2, R-AM, PA12, T19-14x, VT-19-17-H2, RCRE, RCRIP, GP+E-86, GP+envAm12, and DAN cell lines as described in Miller, Human Gene Therapy 1:5-14 (1990), which is incorporated herein by reference. The vector can transduce the packaging cells through any means known in the art. A producer cell line generates infectious retroviral vector particles which include polynucleotide encoding a DNA replication protein. Such retroviral vector particles can then be employed to transduce eukaryotic cells either in vitro or in vivo. The transduced eukaryotic cells will express a DNA replication protein.

Essentially any method for introducing a nucleic acid construct into cells can be employed. Physical methods of introducing nucleic acids include injection of a solution containing the construct, bombardment by particles covered by the construct, soaking a cell, tissue sample or organism in a solution of the nucleic acid, or electroporation of cell membranes in the presence of the construct. A viral construct packaged into a viral particle can be used to accomplish both efficient introduction of an expression construct into the cell and transcription of the encoded shRNA. Other methods known in the art for introducing nucleic acids to cells can be used, such as lipid-mediated carrier transport, chemical mediated transport, such as calcium phosphate, and the like. Thus the shRNA-encoding nucleic acid construct can be introduced along with components that perform one or more of the following activities: enhance RNA uptake by the cell, promote annealing of the duplex strands, stabilize the annealed strands, or otherwise increase inhibition of the target gene.

For expression within cells, DNA vectors, for example plasmid vectors comprising either an RNA polymerase II or RNA polymerase III promoter can be employed. Expression of endogenous miRNAs is controlled by RNA polymerase II (Pol II) promoters and in some cases, shRNAs are most efficiently driven by Pol II promoters, as compared to RNA polymerase III promoters (Dickins et al., Nat. Genet. 39: 914-921 (2005)). In some embodiments, expression of the shRNA can be controlled by an inducible promoter or a conditional expression system, including, without limitation, RNA polymerase type II promoters. Examples of useful promoters in the context of the invention are tetracycline-inducible promoters (including TRE-tight), IPTG-inducible promoters, tetracycline transactivator systems, and reverse tetracycline transactivator (rtTA) systems. Constitutive promoters can also be used, as can cell-or tissue-specific promoters. Many promoters will be ubiquitous, such that they are expressed in all cell and tissue types. A certain embodiment uses tetracycline-responsive promoters, one of the most effective conditional gene expression systems in in vitro and in vivo studies. See International Patent Publication No. WO 2004/029219 A2 and Fewell et al., Drug Discovery Today 11:975-982 (2006) for a description of inducible shRNA.

Antibodies and Antibody Fragments

The antibody, or antibody fragment, can be any monoclonal or polyclonal antibody or antibody fragment that specifically binds to c-Kit and reduces the expression or biological activity of c-Kit. The antibody, or antibody fragment, can be any monoclonal or polyclonal antibody or antibody fragment that specifically binds to a protein that interacts with c-Kit. For example, the antibody or antibody fragment can bind to stem cell factor. The antibody or antibody fragment can bind any molecule that will result in reducing the expression or biological activity of c-Kit.

Methods for preparing polyclonal antibodies are well-known in the art. Polyclonal antibodies can be raised by immunizing an animal (e.g., a rabbit, rat, mouse, donkey, and the like) with multiple subcutaneous or intraperitoneal injections of the relevant antigen (a purified peptide fragment, full-length recombinant protein, fusion protein, and the like) optionally conjugated to keyhole limpet hemocyanin (KLH), serum albumin, and the like, diluted in sterile saline and combined with an adjuvant (e.g. Complete or Incomplete Freund' s Adjuvant) to form a stable emulsion. The polyclonal antibody is then recovered from blood, ascites, and the like, of an animal so immunized. Collected blood is clotted, and the serum decanted, clarified by centrifugation, and assayed for antibody titer. The polyclonal antibodies can be purified from serum or ascites according to standard methods in the art, including, but not limited to, affinity chromatography, ion-exchange chromatography, gel electrophoresis, dialysis.

Monoclonal antibodies can be prepared using hybridoma methods, such as those described by Kohler and Milstein, Nature 256:495 (1975). Using the hybridoma method, a mouse, hamster, or other appropriate host animal, is immunized as described above to elicit the production by lymphocytes of antibodies that will specifically bind to an immunizing antigen. Alternatively, lymphocytes can be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay such as radioimmunoassay (RIA) or enzyme-linked immunosorbent assay (ELISA) can then be propagated either in vitro culture using standard methods (see Goding, Monoclonal Antibodies: Principles and Practice, Academic Press, 1986) or in vivo as ascites tumors in an animal. The monoclonal antibodies can then be purified from the culture medium or ascites fluid as described for polyclonal antibodies above.

Alternatively monoclonal antibodies can also be made using recombinant DNA methods well-known in the art. See, e.g., U.S. Pat. No. 4,816,567. For example, polynucleotides encoding a monoclonal antibody can be isolated from mature B-cells, hybridoma cells, and the like, using RT-PCR oligonucleotide primers that specifically amplify the genes encoding the heavy and light chains of the antibody. The sequence is determined using conventional procedures, and the isolated polynucleotides encoding the heavy and light chains are then cloned into suitable expression vectors. Transfection of the expression vectors into host cells such as E. coli cells, simian COS cells, Chinese hamster ovary (CHO) cells, myeloma cells, and the like, results in generation of the monoclonal antibody of interest by the host cells. Also, recombinant monoclonal antibodies or fragments thereof of the desired species can be isolated from phage display libraries as described in McCafferty et al., Nature 348:552-554 (1990); Clackson et al., Nature 352:624-628 (1991); and Marks et al., J. Mol. Biol. 222:581-597 (1991).

The polynucleotide(s) encoding a monoclonal antibody can further be modified in a number of different manners using recombinant DNA technology to generate alternative antibodies. In embodiments, the constant domains of the light and heavy chains of, for example, a mouse monoclonal antibody can be substituted 1) for those regions of, for example, a human antibody to generate a chimeric antibody, or 2) for a non-immunoglobulin polypeptide to generate a fusion antibody. In embodiments, the constant regions are truncated or removed to generate the desired antibody fragment of a monoclonal antibody. Furthermore, site-directed or high-density mutagenesis of the variable region can be used to optimize specificity, affinity, etc. of a monoclonal antibody.

In embodiments, the monoclonal antibody against c-Kit is a humanized antibody. Humanized antibodies are antibodies that contain minimal sequences from non-human (e.g murine) antibodies within the variable regions. Such antibodies are used therapeutically to reduce antigenicity and HAMA (human anti-mouse antibody) responses when administered to a human subject. In practice, humanized antibodies are typically human antibodies with minimum to no non-human sequences. A human antibody is an antibody produced by a human or an antibody having an amino acid sequence corresponding to an antibody produced by a human.

Humanized antibodies can be produced using various techniques known in the art. An antibody can be humanized by substituting the CDR of a human antibody with that of a non-human antibody (e.g., mouse, rat, rabbit, hamster, and the like) having the desired specificity, affinity, and capability (see Jones et al., Nature 321:522-525 (1986); Riechmann et al., Nature 332:323-327 (1988); and Verhoeyen et al., Science 239:1534-1536 (1988)). The humanized antibody can be further modified by the substitution of additional residues either in the Fv framework region and/or within the replaced non-human residues to refine and optimize antibody specificity, affinity, and/or capability.

Human antibodies can be directly prepared using various techniques well-known in the art. Immortalized human B lymphocytes immunized in vitro or isolated from an immunized individual that produces an antibody directed against a target antigen can be generated (See, e.g., Cole et al., Monoclonal Antibodies and Cancer Therapy, Alan R. Liss, p. 77 (1985); Boemer et al., J. Immunol. 147:86-95 (1991); and U.S. Pat. No. 5,750,373). Also, the human antibody can be selected from a phage library that expresses human antibodies (see Vaughan et al., Nature Biotechnology 14:309-314 (1996); Sheets et al., PNAS 95:6157-6162 (1998); Hoogenboom and Winter, J. Mol. Biol. 227:381 (1991); and Marks et al., J. Mol. Biol. 222:581 (1991)). Human antibodies can also be made in transgenic mice containing human immunoglobulin loci that are capable upon immunization of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

It may further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

The invention further embraces variants and equivalents which are substantially homologous to the chimeric, humanized and human antibodies, or antibody fragments thereof, set forth herein. These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid, or one neutral amino acid by another neutral amino acid. What is intended by a conservative amino acid substitution is well known in the art.

### Methods of Treatment

Methods of the invention address a long felt need for effective treatment against virus-induced respiratory pathology. These methods treat or prevent virus-induced respiratory pathology (e.g., airway inflammation and airway hyperreactivity (AHR)) by administering a c-Kit kinase inhibitor. Not wishing to be bound by any theories, it is thought that the c-Kit kinase inhibitor prevents activation of nuocytes in the pulmonary tract, thereby reducing pulmonary inflammation.

Thus, in one aspect, the invention provides methods for preventing or treating virus-induced respiratory pathology in a subject. The methods involve administering an effective amount of a c-Kit kinase inhibitor to the subject.

In another aspect, the invention provides methods for preventing or treating virus-induced airway inflammation in a subject. In certain embodiments, the airway inflammation is associated with asthma. The methods involve administering an effective amount of a c-Kit kinase inhibitor to the subject.

In another aspect, the invention provides methods for preventing or treating virus-induced AHR in a subject. The methods involve administering an effective amount of a c-Kit kinase inhibitor to the subject.

In another aspect, the invention provides methods for preventing or treating respiratory pathology in a subject. The methods involve administering to a subject having or at risk of developing a viral infection a c-Kit kinase inhibitor, thereby preventing or treating respiratory pathology in the subject.

In another aspect, the invention provides methods for preventing or treating airway inflammation in a subject. The methods involve administering to a subject having or at risk of developing a viral infection a c-Kit kinase inhibitor, thereby preventing or treating airway inflammation in the subject.

In another aspect, the invention provides methods for preventing or treating AHR in a subject. The methods involve administering to a subject having or at risk of developing a viral infection a c-Kit kinase inhibitor, thereby preventing or treating AHR in the subject.

In another aspect, the invention provides methods for inhibiting nuocyte activation in a subject. The methods involve administering to a subject having or at risk of developing a viral infection a c-Kit kinase inhibitor, thereby inhibiting nuocyte activation in the subject.

In another aspect, the invention provides methods for inhibiting lymphokine production in a subject. The methods involve administering to a subject having or at risk of developing a viral infection a c-Kit kinase inhibitor, thereby inhibiting lymphokine production in the subject. In embodiments, the lymphokine is IL-13 or IL-5.

In another aspect, the invention provides methods for inhibiting nuocyte activation. The methods involve contacting a nuocyte with a c-Kit kinase inhibitor, thereby inhibiting nuocyte activation.

In another aspect, the invention provides methods for inhibiting lymphokine production in a cell (e.g., nuocyte). The methods involve contacting a cell (e.g., nuocyte) with a c-Kit kinase inhibitor, thereby inhibiting lymphokine production by the cell.

In embodiments, the virus is any virus that causes asthma symptoms (e.g., respiratory syncytial virus (RSV), influenza, rhinovirus, parainfluenza, adenovirus, coronavirus, metapneumovirus, bocavirus, and the like). In embodiments, the viral infection is infection by a virus that causes asthma symptoms (e.g., respiratory syncytial virus (RSV), influenza, rhinovirus, parainfluenza, adenovirus, coronavirus, metapneumovirus, or bocavirus).

In embodiments, the subject is a mammal, (e.g., human). In related embodiments, the subject is susceptible to viral infection (e.g., a pregnant female, a young child, an infant, an elderly subject, or a person having an underlying medical condition/pre-existing condition). In certain embodiments, the subject has asthma.

In embodiments, the viral infection is an acute viral infection. In related embodiments, the c-Kit kinase inhibitor is administered to the subject for 1-7 days, 1-5 days, or 1-3 days.

In embodiments, the subject does not respond to corticosteroid therapy.

In embodiments, a cell (e.g., a nuocyte) is contacted with the c-Kit kinase inhibitor for 1-7 days, 1-5 days, or 1-3 days.

In embodiments, the methods involve administering at least one additional anti-asthma medication to the subject.

In embodiments, the methods involve contacting a cell (e.g., a nuocyte) with at least one additional anti-asthma medication to the subject.

The additional anti-asthma medication can be any anti-asthma medication known in the art. For example, the additional anti-asthma medication can be at least one other c-kit inhibitor. The additional anti-asthma medication can also be a corticosteroid, a beta-agonist, a leukotriene modifier, a mast cell stabilizer, theophylline, an immunomodulator, an anti-IgE therapy (e.g., omalizumab), or an anti-cholinergic. The additional anti-asthma medication can be administered with the intention of immediate relief of respiratory symptoms, or as a (long term/continuous) control for respiratory symptoms.

Methods for evaluating the therapeutic efficacy of the methods of the invention are standard in the art. For example, efficacy of treatment can be evaluated by assessing NO levels, blood oxygen levels, patient symptoms (e.g., shortness of breath, chest tightness, coughing, or wheezing), and the like.

### Pharmaceutical Compositions

The invention provides for pharmaceutical compositions containing at least one agent that reduces the expression and/or biological activity of c-Kit. The agent can be any one of such agents described herein. In embodiments, the pharmaceutical compositions contain a pharmaceutically acceptable carrier, excipient, or diluent, which includes any pharmaceutical agent that does not itself induce the production of an immune response harmful to a subject receiving the composition, and which may be administered without undue toxicity. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful for treating and/or preventing virus-induced respiratory pathology (e.g., airway inflammation and/or airway hypersensitivity (AHR)).

A thorough discussion of pharmaceutically acceptable carriers, diluents, and other excipients is presented in Remington's Pharmaceutical Sciences (17th ed., Mack Publishing Company) and Remington: The Science and Practice of Pharmacy (21st ed., Lippincott Williams & Wilkins), which are hereby incorporated by reference. The formulation of the pharmaceutical composition should suit the mode of administration. In embodiments, the pharmaceutical composition is suitable for administration to humans, and can be sterile, non-particulate and/or non-pyrogenic.

Pharmaceutically acceptable carriers, excipients, or diluents include, but are not limited, to saline, buffered saline, dextrose, water, glycerol, ethanol, sterile isotonic aqueous buffer, and combinations thereof.

Wetting agents, emulsifiers and lubricants, such as sodium lauryl sulfate and magnesium stearate, as well as coloring agents, release agents, coating agents, sweetening, flavoring and perfuming agents, preservatives, and antioxidants can also be present in the compositions.

Examples of pharmaceutically-acceptable antioxidants include, but are not limited to: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In embodiments, the pharmaceutical composition is provided in a solid form, such as a lyophilized powder suitable for reconstitution, a liquid solution, suspension, emulsion, tablet, pill, capsule, sustained release formulation, or powder.

In embodiments, the pharmaceutical composition is supplied in liquid form, for example, in a sealed container indicating the quantity and concentration of the active ingredient in the pharmaceutical composition. In related embodiments, the liquid form of the pharmaceutical composition is supplied in a hermetically sealed container.

Methods for formulating the pharmaceutical compositions of the present invention are conventional and well-known in the art (see Remington and Remington's). One of skill in the art can readily formulate a pharmaceutical composition having the desired characteristics (e.g., route of administration, biosafety, and release profile).

Methods for preparing the pharmaceutical compositions include the step of bringing into association the active ingredient with a pharmaceutically acceptable carrier and, optionally, one or more accessory ingredients. The pharmaceutical compositions can be prepared by uniformly and intimately bringing into association the active ingredient with liquid carriers, or finely divided solid carriers, or both, and then, if necessary, shaping the product. Additional methodology for preparing the pharmaceutical compositions, including the preparation of multilayer dosage forms, are described in Ansel's Pharmaceutical Dosage Forms and Drug Delivery Systems (9th ed., Lippincott Williams & Wilkins), which is hereby incorporated by reference.

### Methods of Delivery

The pharmaceutical compositions of the invention can be administered to a subject by oral and non-oral means (e.g., topically, transdermally, or by injection). Such modes of administration and the methods for preparing an appropriate pharmaceutical composition for use therein are described in Gibaldi's Drug Delivery Systems in Pharmaceutical Care (1st ed., American Society of Health-System Pharmacists), which is hereby incorporated by reference.

In embodiments, the pharmaceutical compositions are administered orally in a solid form.

Pharmaceutical compositions suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound(s) described herein, a derivative thereof, or a pharmaceutically acceptable salt or prodrug thereof as the active ingredient(s). The active ingredient can also be administered as a bolus, electuary, or paste.

In solid dosage forms for oral administration (e.g., capsules, tablets, pills, dragees, powders, granules and the like), the active ingredient is mixed with one or more pharmaceutically acceptable carriers, excipients, or diluents, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets, and pills, the pharmaceutical compositions can also comprise buffering agents. Solid compositions of a similar type can also be prepared using fillers in soft and hard-filled gelatin capsules, and excipients such as lactose or milk sugars, as well as high molecular weight polyethylene glycols and the like.

A tablet can be made by compression or molding, optionally with one or more accessory ingredients. Compressed tablets can be prepared using binders (for example, gelatin or hydroxypropylmethyl cellulose), lubricants, inert diluents, preservatives, disintegrants (for example, sodium starch glycolate or cross-linked sodium carboxymethyl cellulose), surface-actives, and/ or dispersing agents. Molded tablets can be made by molding in a suitable machine a mixture of the powdered active ingredient moistened with an inert liquid diluent.

The tablets and other solid dosage forms, such as dragees, capsules, pills, and granules, can optionally be scored or prepared with coatings and shells, such as enteric coatings and other coatings well-known in the art.

The pharmaceutical compositions can also be formulated so as to provide slow, extended, or controlled release of the active ingredient therein using, for example, hydroxypropylmethyl cellulose in varying proportions to provide the desired release profile, other polymer matrices, liposomes and/or microspheres. The pharmaceutical compositions can also optionally contain opacifying agents and may be of a composition that releases the active ingredient(s) only, or preferentially, in a certain portion of the gastrointestinal tract, optionally, in a delayed manner. Examples of embedding compositions include polymeric substances and waxes. The active ingredient can also be in micro-encapsulated form, if appropriate, with one or more pharmaceutically acceptable carriers, excipients, or diluents well-known in the art (see, e.g., Remington and Remington's).

The pharmaceutical compositions can be sterilized by, for example, filtration through a bacteria-retaining filter, or by incorporating sterilizing agents in the form of sterile solid compositions which can be dissolved in sterile water, or some other sterile injectable medium immediately before use.

In embodiments, the pharmaceutical compositions are administered orally in a liquid form.

Liquid dosage forms for oral administration of an active ingredient include pharmaceutically acceptable emulsions, microemulsions, solutions, suspensions, syrups and elixirs. In addition to the active ingredient, the liquid dosage forms can contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. In addition to inert diluents, the liquid pharmaceutical compositions can include adjuvants such as wetting agents, emulsifying and suspending agents, sweetening, flavoring, coloring, perfuming and preservative agents, and the like.

Suspensions, in addition to the active ingredient(s) can contain suspending agents such as, but not limited to, ethoxylated isostearyl alcohols, polyoxyethylene sorbitol and sorbitan esters, microcrystalline cellulose, aluminum metahydroxide, bentonite, agar-agar and tragacanth, and mixtures thereof.

In embodiments, the pharmaceutical compositions are administered by non-oral means such as by topical application, transdermal application, injection, and the like. In related embodiments, the pharmaceutical compositions are administered parenterally by injection, infusion, or implantation (e.g., intravenous, intramuscular, intraarticular, subcutaneous, and the like).

Compositions for parenteral use can be presented in unit dosage forms, e.g. in ampoules or in vials containing several doses, and in which a suitable preservative can be added. Such compositions can be in form of a solution, a suspension, an emulsion, an infusion device, a delivery device for implantation, or it can be presented as a dry powder to be reconstituted with water or another suitable vehicle before use. One or more co-vehicles, such as ethanol, can also be employed. Apart from the active ingredient(s), the compositions can contain suitable parenterally acceptable carriers and/or excipients or the active ingredient(s) can be incorporated into microspheres, microcapsules, nanoparticles, liposomes, or the like for controlled release. Furthermore, the compositions can also contain suspending, solubilising, stabilising, pH-adjusting agents, and/or dispersing agents.

The pharmaceutical compositions can be in the form of sterile injections. To prepare such a composition, the active ingredient is dissolved or suspended in a parenterally acceptable liquid vehicle. Exemplary vehicles and solvents include, but are not limited to, water, water adjusted to a suitable pH by addition of an appropriate amount of hydrochloric acid, sodium hydroxide or a suitable buffer, 1,3-butanediol, Ringer's solution and isotonic sodium chloride solution. The pharmaceutical composition can also contain one or more preservatives, for example, methyl, ethyl or n-propyl p-hydroxybenzoate. To improve solubility, a dissolution enhancing or solubilising agent can be added or the solvent can contain 10-60% w/w of propylene glycol or the like.

The pharmaceutical compositions can contain one or more pharmaceutically acceptable sterile isotonic aqueous or nonaqueous solutions, dispersions, suspensions or emulsions, or sterile powders, which can be reconstituted into sterile injectable solutions or dispersions just prior to use. Such pharmaceutical compositions can contain antioxidants; buffers; bacteriostats; solutes, which render the formulation isotonic with the blood of the intended recipient; suspending agents; thickening agents; preservatives; and the like.

Examples of suitable aqueous and nonaqueous carriers, which can be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In some embodiments, in order to prolong the effect of an active ingredient, it is desirable to slow the absorption of the compound from subcutaneous or intramuscular injection. This can be accomplished by the use of a liquid suspension of crystalline or amorphous material having poor water solubility. The rate of absorption of the active ingredient then depends upon its rate of dissolution which, in turn, can depend upon crystal size and crystalline form. Alternatively, delayed absorption of a parenterally-administered active ingredient is accomplished by dissolving or suspending the compound in an oil vehicle. In addition, prolonged absorption of the injectable pharmaceutical form can be brought about by the inclusion of agents that delay absorption such as aluminum monostearate and gelatin.

Controlled release parenteral compositions can be in form of aqueous suspensions, microspheres, microcapsules, magnetic microspheres, oil solutions, oil suspensions, emulsions, or the active ingredient can be incorporated in biocompatible carrier(s), liposomes, nanoparticles, implants or infusion devices.

Materials for use in the preparation of microspheres and/or microcapsules include biodegradable/bioerodible polymers such as polyglactin, poly-(isobutyl cyanoacrylate), poly(2-hydroxyethyl-L-glutamine) and poly(lactic acid).

Biocompatible carriers which can be used when formulating a controlled release parenteral formulation include carbohydrates such as dextrans, proteins such as albumin, lipoproteins or antibodies.

Materials for use in implants can be non-biodegradable, e.g., polydimethylsiloxane, or biodegradable such as, e.g., poly(caprolactone), poly(lactic acid), poly(glycolic acid) or poly(ortho esters).

In embodiments, the active ingredient(s) are administered by aerosol. This is accomplished by preparing an aqueous aerosol, liposomal preparation, or solid particles containing the compound. A nonaqueous (e.g., fluorocarbon propellant) suspension can be used. The pharmaceutical composition can also be administered using a sonic nebulizer, which would minimize exposing the agent to shear, which can result in degradation of the compound.

Ordinarily, an aqueous aerosol is made by formulating an aqueous solution or suspension of the active ingredient(s) together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Dosage forms for topical or transdermal administration of an active ingredient(s) includes powders, sprays, ointments, pastes, creams, lotions, gels, solutions, patches and inhalants. The active ingredient(s) can be mixed under sterile conditions with a pharmaceutically acceptable carrier, and with any preservatives, buffers, or propellants as appropriate.

Transdermal patches suitable for use in the present invention are disclosed in Transdermal Drug Delivery: Developmental Issues and Research Initiatives (Marcel Dekker Inc., 1989) and U.S. Pat. Nos. 4,743,249, 4,906,169, 5,198,223, 4,816,540, 5,422,119, 5,023,084, which are hereby incorporated by reference. The transdermal patch can also be any transdermal patch well-known in the art, including transscrotal patches. Pharmaceutical compositions in such transdermal patches can contain one or more absorption enhancers or skin permeation enhancers well-known in the art (see, e.g., U.S. Pat. Nos. 4,379,454 and 4,973,468, which are hereby incorporated by reference). Transdermal therapeutic systems for use in the present invention can be based on iontophoresis, diffusion, or a combination of these two effects.

Transdermal patches have the added advantage of providing controlled delivery of active ingredient(s) to the body. Such dosage forms can be made by dissolving or dispersing the active ingredient(s) in a proper medium. Absorption enhancers can also be used to increase the flux of the active ingredient across the skin. The rate of such flux can be controlled by either providing a rate controlling membrane or dispersing the active ingredient(s) in a polymer matrix or gel.

Such pharmaceutical compositions can be in the form of creams, ointments, lotions, liniments, gels, hydrogels, solutions, suspensions, sticks, sprays, pastes, plasters and other kinds of transdermal drug delivery systems. The compositions can also include pharmaceutically acceptable carriers or excipients such as emulsifying agents, antioxidants, buffering agents, preservatives, humectants, penetration enhancers, chelating agents, gel-forming agents, ointment bases, perfumes, and skin protective agents.

Examples of emulsifying agents include, but are not limited to, naturally occurring gums, e.g. gum acacia or gum tragacanth, naturally occurring phosphatides, e.g. soybean lecithin and sorbitan monooleate derivatives.

Examples of antioxidants include, but are not limited to, butylated hydroxy anisole (BHA), ascorbic acid and derivatives thereof, tocopherol and derivatives thereof, and cysteine.

Examples of preservatives include, but are not limited to, parabens, such as methyl or propyl p-hydroxybenzoate and benzalkonium chloride.

Examples of humectants include, but are not limited to, glycerin, propylene glycol, sorbitol and urea.

Examples of penetration enhancers include, but are not limited to, propylene glycol, DMSO, triethanolamine, N,N-dimethylacetamide, N,N-dimethylformamide, 2-pyrrolidone and derivatives thereof, tetrahydrofurfuryl alcohol, propylene glycol, diethylene glycol monoethyl or monomethyl ether with propylene glycol monolaurate or methyl laurate, eucalyptol, lecithin, Transcutol®, and Azone®.

Examples of chelating agents include, but are not limited to, sodium EDTA, citric acid and phosphoric acid.

Examples of gel forming agents include, but are not limited to, Carbopol, cellulose derivatives, bentonite, alginates, gelatin and polyvinylpyrrolidone.

In addition to the active ingredient(s), the ointments, pastes, creams, and gels of the present invention can contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain excipients such as lactose, talc, silicic acid, aluminum hydroxide, calcium silicates and polyamide powder, or mixtures of these substances. Sprays can additionally contain customary propellants, such as chlorofluorohydrocarbons, and volatile unsubstituted hydrocarbons, such as butane and propane.

Injectable depot forms are made by forming microencapsule matrices of compound(s) of the invention in biodegradable polymers such as polylactide-polyglycolide. Depending on the ratio of compound to polymer, and the nature of the particular polymer employed, the rate of compound release can be controlled. Examples of other biodegradable polymers include poly(orthoesters) and poly(anhydrides). Depot injectable formulations are also prepared by entrapping the drug in liposomes or microemulsions which are compatible with body tissue.

Subcutaneous implants are well-known in the art and are suitable for use in the present invention. Subcutaneous implantation methods are preferably non-irritating and mechanically resilient. The implants can be of matrix type, of reservoir type, or hybrids thereof. In matrix type devices, the carrier material can be porous or non-porous, solid or semi-solid, and permeable or impermeable to the active compound or compounds. The carrier material can be biodegradable or may slowly erode after administration. In some instances, the matrix is non-degradable but instead relies on the diffusion of the active compound through the matrix for the carrier material to degrade. Alternative subcutaneous implant methods utilize reservoir devices where the active compound or compounds are surrounded by a rate controlling membrane, e.g., a membrane independent of component concentration (possessing zero-order kinetics). Devices consisting of a matrix surrounded by a rate controlling membrane also suitable for use.

Both reservoir and matrix type devices can contain materials such as polydimethylsiloxane, such as Silastic™, or other silicone rubbers. Matrix materials can be insoluble polypropylene, polyethylene, polyvinyl chloride, ethylvinyl acetate, polystyrene and polymethacrylate, as well as glycerol esters of the glycerol palmitostearate, glycerol stearate, and glycerol behenate type. Materials can be hydrophobic or hydrophilic polymers and optionally contain solubilising agents.

Subcutaneous implant devices can be slow-release capsules made with any suitable polymer, e.g., as described in U.S. Pat. Nos. 5,035,891 and 4,210,644, which are hereby incorporated by reference.

In general, at least four different approaches are applicable in order to provide rate control over the release and transdermal permeation of a drug compound. These approaches are: membrane-moderated systems, adhesive diffusion-controlled systems, matrix dispersion-type systems and microreservoir systems. It is appreciated that a controlled release percutaneous and/or topical composition can be obtained by using a suitable mixture of these approaches.

In a membrane-moderated system, the active ingredient is present in a reservoir which is totally encapsulated in a shallow compartment molded from a drug-impermeable laminate, such as a metallic plastic laminate, and a rate-controlling polymeric membrane such as a microporous or a non-porous polymeric membrane, e.g., ethylene-vinyl acetate copolymer. The active ingredient is released through the rate controlling polymeric membrane. In the drug reservoir, the active ingredient can either be dispersed in a solid polymer matrix or suspended in an unleachable, viscous liquid medium such as silicone fluid. On the external surface of the polymeric membrane, a thin layer of an adhesive polymer is applied to achieve an intimate contact of the transdermal system with the skin surface. The adhesive polymer is preferably a polymer which is hypoallergenic and compatible with the active drug substance.

In an adhesive diffusion-controlled system, a reservoir of the active ingredient is formed by directly dispersing the active ingredient in an adhesive polymer and then by, e.g., solvent casting, spreading the adhesive containing the active ingredient onto a flat sheet of substantially drug-impermeable metallic plastic backing to form a thin drug reservoir layer.

A matrix dispersion-type system is characterized in that a reservoir of the active ingredient is formed by substantially homogeneously dispersing the active ingredient in a hydrophilic or lipophilic polymer matrix. The drug-containing polymer is then molded into disc with a substantially well-defined surface area and controlled thickness. The adhesive polymer is spread along the circumference to form a strip of adhesive around the disc.

A microreservoir system can be considered as a combination of the reservoir and matrix dispersion type systems. In this case, the reservoir of the active substance is formed by first suspending the drug solids in an aqueous solution of water-soluble polymer and then dispersing the drug suspension in a lipophilic polymer to form a multiplicity of unleachable, microscopic spheres of drug reservoirs.

Any of the above-described controlled release, extended release, and sustained release compositions can be formulated to release the active ingredient in about 30 minutes to about 1 week, in about 30 minutes to about 72 hours, in about 30 minutes to 24 hours, in about 30 minutes to 12 hours, in about 30 minutes to 6 hours, in about 30 minutes to 4 hours, and in about 3 hours to 10 hours. In embodiments, an effective concentration of the active ingredient(s) is sustained in a subject for 4 hours, 6 hours, 8 hours, 10 hours, 12 hours, 16 hours, 24 hours, 48 hours, 72 hours, or more after administration of the pharmaceutical compositions to the subject.

### Dosages

When the agents described herein are administered as pharmaceuticals to humans and animals, they can be given per se or as a pharmaceutical composition containing active ingredient in combination with a pharmaceutically acceptable carrier, excipient, or diluent.

Actual dosage levels and time course of administration of the active ingredients in the pharmaceutical compositions of the invention can be varied so as to obtain an amount of the active ingredient which is effective to achieve the desired therapeutic response for a particular patient, composition, and mode of administration, without being toxic to the patient. Generally, agents or pharmaceutical compositions of the invention are administered in an amount sufficient to reduce or eliminate virus-induced respiratory pathology (e.g., airway inflammation and/or airway hyperreactivity (AHR)).

Exemplary dose ranges include 0.01 mg to 250 mg per day, 0.01 mg to 100 mg per day, 1 mg to 100 mg per day, 10 mg to 100 mg per day, 1 mg to 10 mg per day, and 0.01 mg to 10 mg per day. A preferred dose of an agent is the maximum that a patient can tolerate and not develop serious or unacceptable side effects. In embodiments, the agent is administered at a concentration of about 10 micrograms to about 100 mg per kilogram of body weight per day, about 0.1 to about 10 mg/kg per day, or about 1.0 mg to about 10 mg/kg of body weight per day.

In embodiments, the pharmaceutical composition comprises an agent in an amount ranging between 1 and 10 mg, such as 1, 2, 3, 4, 5, 6, 7, 8, 9, or 10 mg.

In embodiments, the therapeutically effective dosage produces a serum concentration of an agent of from about 0.1 ng/ml to about 50-100 μg/ml. The pharmaceutical compositions typically should provide a dosage of from about 0.001 mg to about 2000 mg of compound per kilogram of body weight per day. For example, dosages for systemic administration to a human patient can range from 1-10 μg/kg, 20-80 μg/kg, 5-50 μg/kg, 75-150 μg/kg, 100-500 μg/kg, 250-750 μg/kg, 500-1000 μg/kg, 1-10 mg/kg, 5-50 mg/kg, 25-75 mg/kg, 50-100 mg/kg, 100-250 mg/kg, 50-100 mg/kg, 250-500 mg/kg, 500-750 mg/kg, 750-1000 mg/kg, 1000-1500 mg/kg, 1500-2000 mg/kg, 5 mg/kg, 20 mg/kg, 50 mg/kg, 100 mg/kg, 500 mg/kg, 1000 mg/kg, 1500 mg/kg, or 2000 mg/kg. Pharmaceutical dosage unit forms are prepared to provide from about 1 mg to about 5000 mg, for example from about 100 to about 2500 mg of the compound or a combination of essential ingredients per dosage unit form.

In embodiments, about 50 nM to about 1 μM of an agent is administered to a subject. In related embodiments, about 50-100 nM, 50-250 nM, 100-500 nM, 250-500 nM, 250-750 nM, 500-750 nM, 500 nM to 1 μM, or 750 nM to 1 μM of an agent is administered to a subject.

Determination of an effective amount is well within the capability of those skilled in the art, especially in light of the detailed disclosure provided herein. Generally, an efficacious or effective amount of an agent is determined by first administering a low dose of the agent(s) and then incrementally increasing the administered dose or dosages until a desired effect (e.g., reduced respiratory pathology) is observed in the treated subject, with minimal or acceptable toxic side effects. Applicable methods for determining an appropriate dose and dosing schedule for administration of a pharmaceutical composition of the present invention are described, for example, in Goodman and Gilman's The Pharmacological Basis of Therapeutics, Goodman et al., eds., 11th Edition, McGraw-Hill 2005, and Remington: The Science and Practice of Pharmacy, 20th and 21st Editions, Gennaro and University of the Sciences in Philadelphia, Eds., Lippencott Williams & Wilkins (2003 and 2005), each of which is hereby incorporated by reference.

### Combination Therapies

The agents and pharmaceutical compositions described herein can also be administered in combination with another therapeutic molecule. The therapeutic molecule can be any compound used to treat asthma, airway inflammation, airway hyperreactivity (AHR), and the like. Examples of such compounds include, but are not limited to, additional c-Kit kinase inhibitors, corticosteroids, beta-agonists, leukotriene modifiers, mast cell stabilizers, theophylline, immunomodulator, anti-IgE therapy (e.g., omalizumab), and anti-cholinergics.

In aspects of the invention, the c-Kit kinase inhibitor is administered in combination with at least one corticosteroid.

The c-Kit kinase inhibitor can be administered before, during, or after administration of the additional therapeutic agent. In embodiments, the c-Kit kinase inhibitor is administered before the first administration of the additional therapeutic agent. In embodiments, the c-Kit kinase inhibitor is administered after the first administration of the additional therapeutic agent (e.g., 1, 2, 3, 4, 5, 6, 7, 8, 9, 10, 11, 12, 13, 14 days or more). In embodiments, the c-Kit kinase inhibitor is administered simultaneously with the first administration of the additional therapeutic agent.

The amount of therapeutic agent administered to a subject can readily be determined by the attending physician or veterinarian. Generally, an efficacious or effective amount of a c-Kit kinase inhibitor and an additional therapeutic is determined by first administering a low dose of one or both active agents and then incrementally increasing the administered dose or dosages until a desired effect is observed (e.g., reduced respiratory pathology), with minimal or no toxic side effects. Applicable methods for determining an appropriate dose and dosing schedule for administration of a combination of the present invention are described, for example, in Goodman and Gilman's The Pharmacological Basis of Therapeutics, 11th Edition., supra, and in Remington: The Science and Practice of Pharmacy, 20th and 21st Editions, supra.

### Kits

The invention provides for kits for preventing or treating virus-induced respiratory pathology; preventing or treating virus-induced airway inflammation; preventing or treating virus-induced airway hyperreactivity (AHR); inhibiting nuocyte activation; as well as inhibiting lymphokine production. In embodiments, the kit contains one or more agents or pharmaceutical compositions described herein. In embodiments, the kit provides instructions for use. The instructions for use can pertain to any of the methods described herein. In related embodiments, the instructions pertain to using the agent(s) or pharmaceutical composition(s) for treating or preventing virus-induced respiratory pathology (e.g., airway inflammation and/or AHR). Kits according to this aspect of the invention may comprise a carrier means, such as a box, carton, tube or the like, having in close confinement therein one or more container means, such as vials, tubes, ampules, bottles and the like. In embodiments, the kit provides a notice in the form prescribed by a governmental agency regulating the manufacture, use, or sale of pharmaceuticals or biological products, which notice reflects approval by the agency of manufacture, use or sale of the kit and the components therein for human administration.

### EXAMPLES

It should be appreciated that the invention should not be construed to be limited to the examples that are now described; rather, the invention should be construed to include any and all applications provided herein and all equivalent variations within the skill of the ordinary artisan.

### Example 1

### Imatinib Inhibits H3N1 Infection-Caused AHR and Inflammation

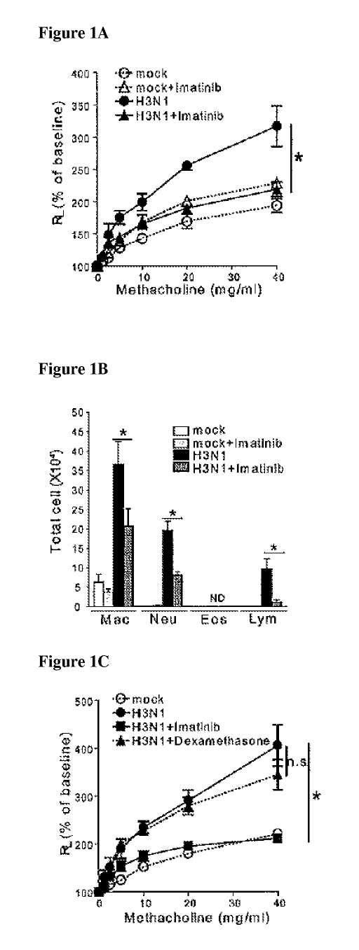

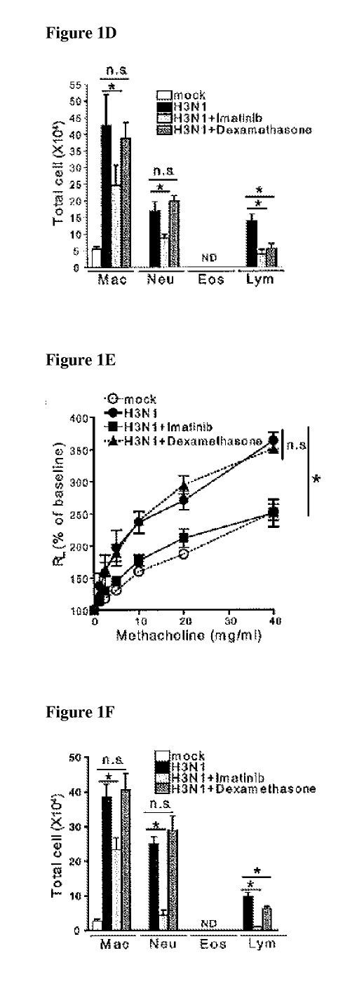

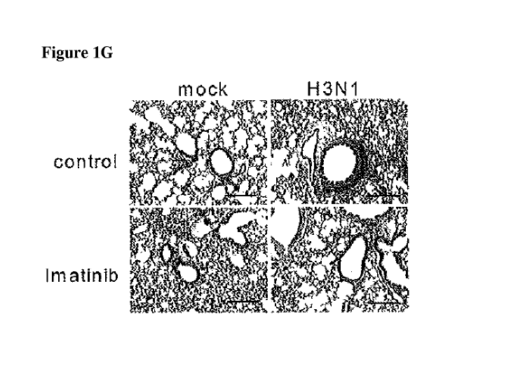

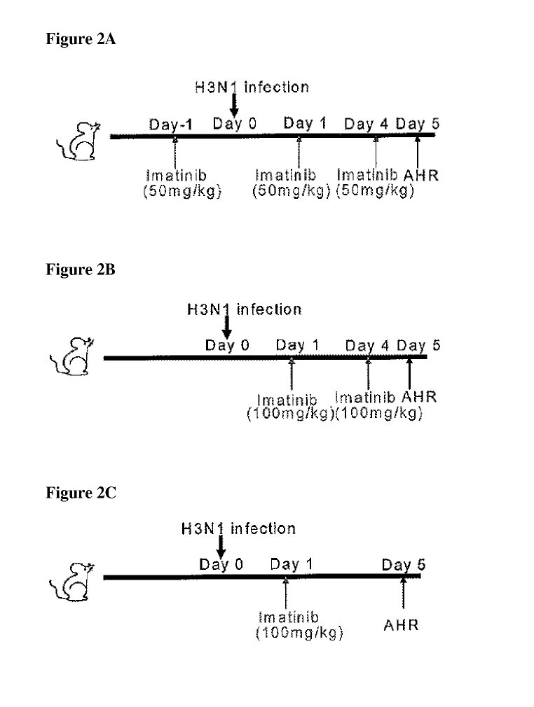

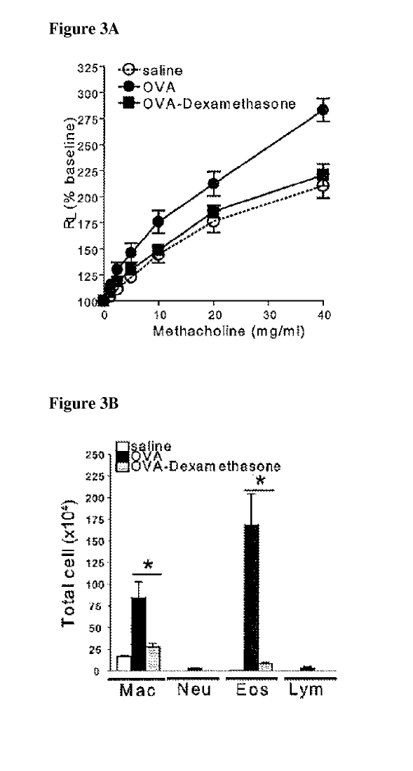

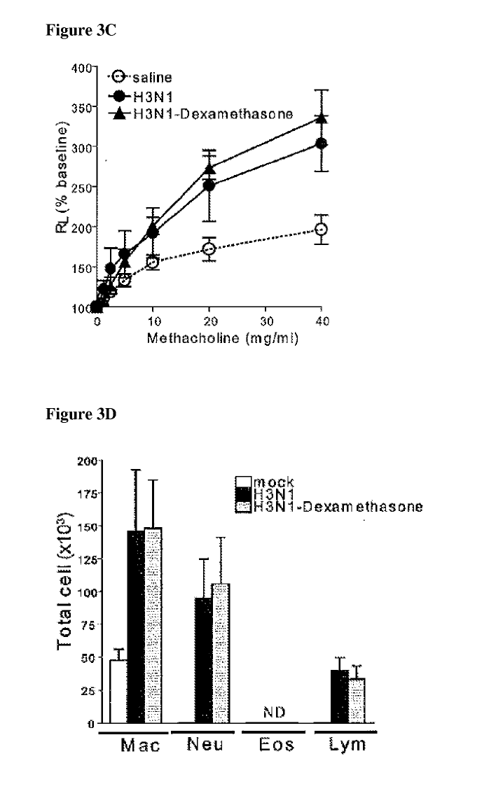

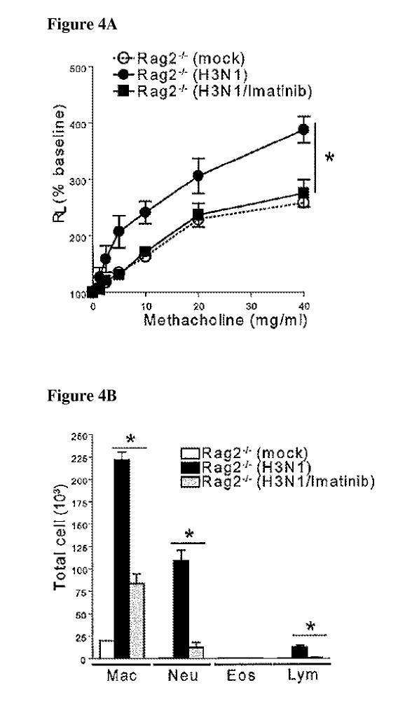

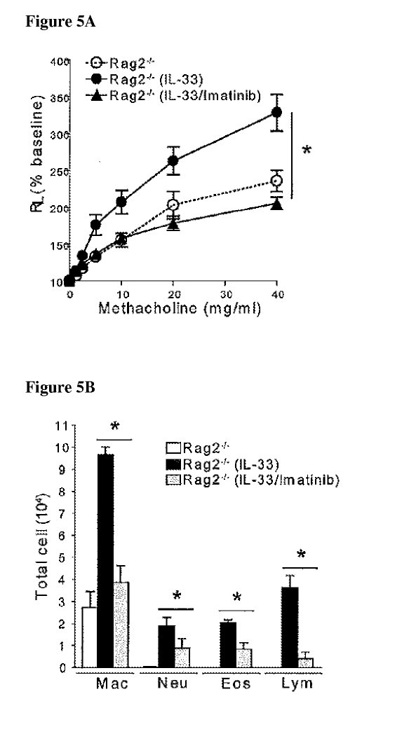

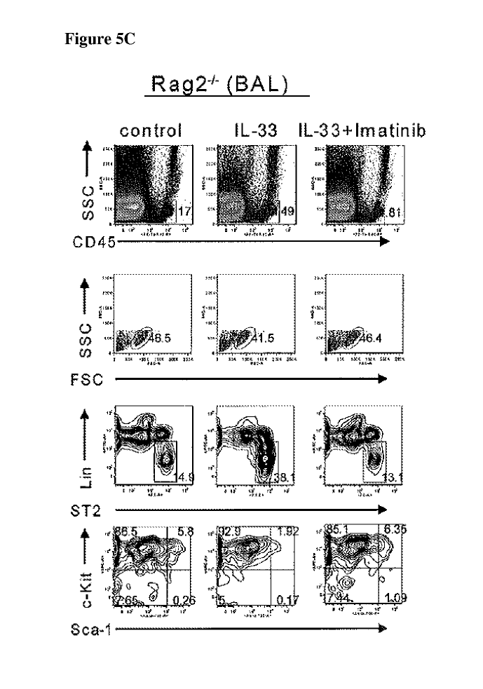

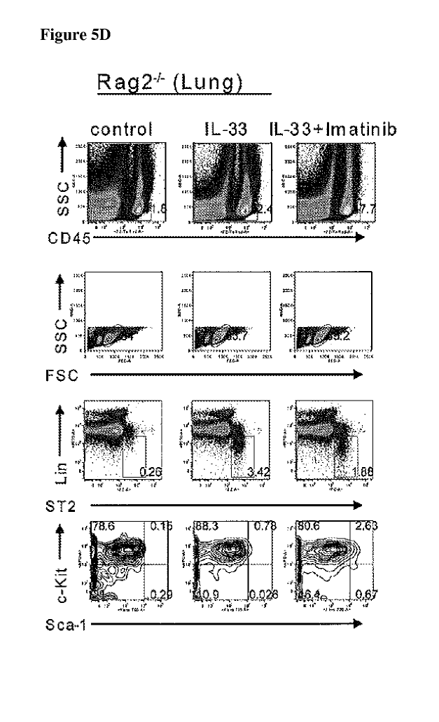

Infection with influenza A virus results in rapid development of AHR, peaking on day 5, driven by a newly defined innate lymphoid cell type called nuocytes, independent of adaptive immunity (Chang, Y. J. et al., Nature Immunol. 12:631-638 (2011)). To develop a therapeutic approach for influenza virus triggered acute asthma, it was determined whether nuocyte function could be blocked by using imatinib (Gleevec; 50 mg/kg). Nuocytes express c-Kit, and imatinib is an inhibitor of c-Kit kinase. The inventors have surprisingly discovered that treatment before and during influenza infection abolished the development of AHR and lung inflammation (FIGS. 1A, 1B, and 2). Furthermore, treatment with imatinib (100 mg/kg, twice) even after the initiation of H3N1 infection abolished AHR and inflammation (FIGS. 1C and 1D). Even a single dose on day 1 post-infection blocked H3N1-induced AHR (FIGS. 1E-1G). In contrast, treatment with corticosteroids, which has been shown to suppress allergen-induced asthma, failed to prevent influenza-induced AHR (FIGS. 1C, 1E, and 3). Suppression of AHR by imatinib did not depend on T cells or B cells since imatinib blocked influenza-induced AHR in Rag2^{-/-} mice (FIG. 4). It is unlikely that imatinib abolished AHR by reducing influenza-induced IL-33 production, since imatinib could block influenza-induced AHR and inflammation that was induced with recombinant IL-33 (rIL-33) in Rag2^{-/-} mice (FIGS. 5A and 5B). These results indicate that imatinib blocks influenza-induced AHR by targeting nuocytes, and that nuocyte function is relatively resistant to corticosteroids.

### Example 2

### Imatinib Reduces the Number of Lung Nuocytes

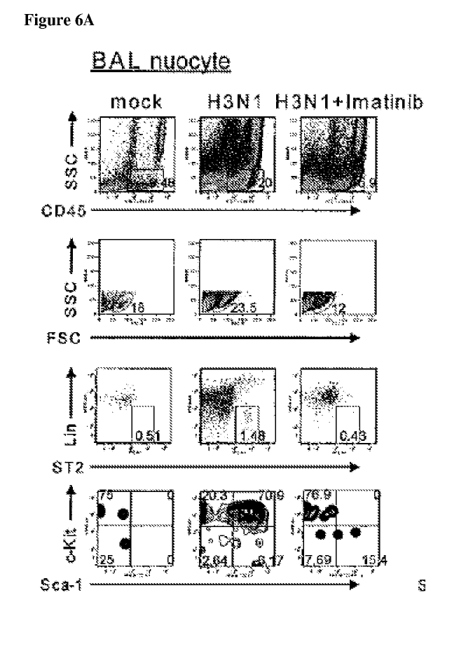

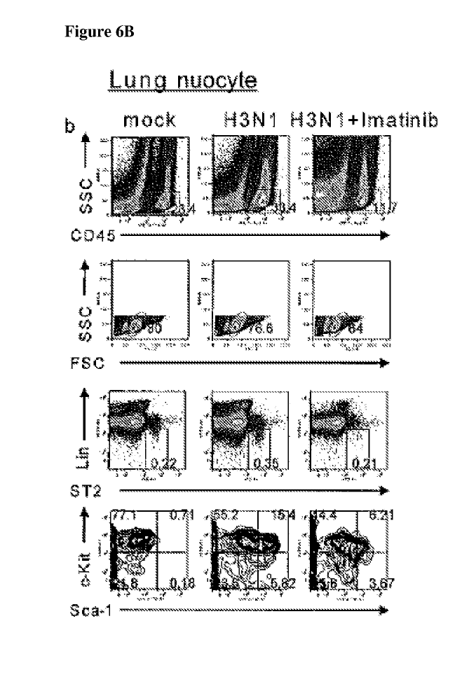

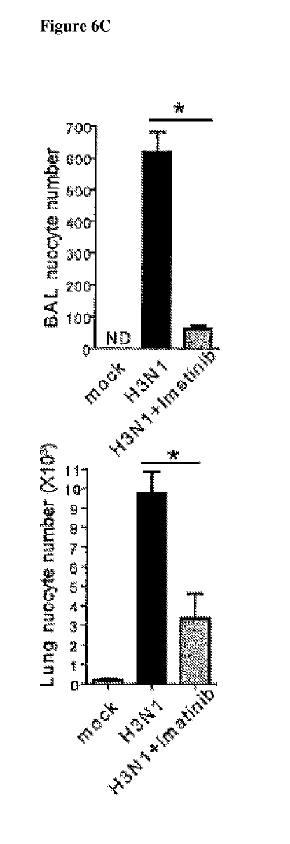

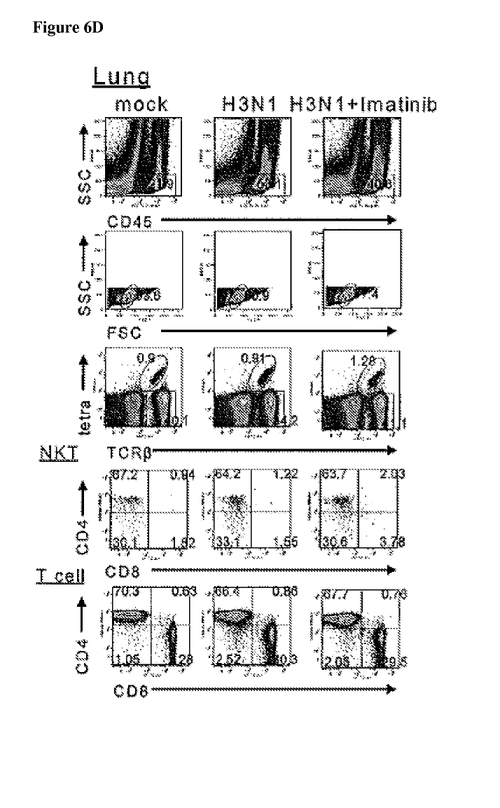

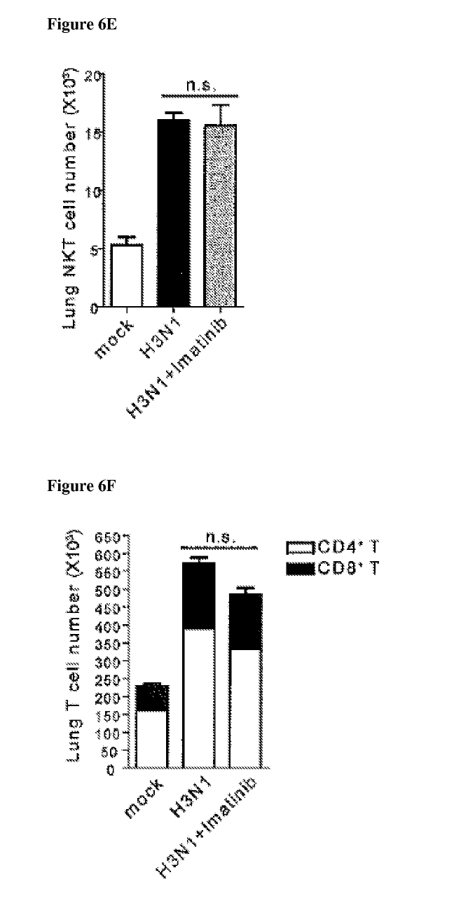

To further examine the effects of imatinib on nuocytes, the frequency of nuocytes (CD45⁺Lin⁻ST2⁺cKit⁺Sca1⁺) in bronchoalveolar lavage (BAL) fluid and lungs of mice after infection were assessed. Treatment of mice with imatinib (100 mg/kg) on day 1 reduced the frequency and absolute number of nuocytes in BAL fluid (FIG. 6A) and in the lungs on day 5 (FIGS. 6B and 6C). In contrast, treatment with imatinib had no effect on the total number of NKT cells and conventional T cells in the lung on day 5 (FIGS. 6D-6F), although the frequency of NKT cells in BAL fluid was slightly reduced as was the total number of BAL fluid cells after imatinib treatment (data not shown). Imatinib did not reduce the number of lung nuocytes by affecting IL-33 production, since imatinib treatment also reduced the number of BAL and lung nuocytes when airway nuocytophilia was induced in Rag2^{-/-} mice with rIL-33 (FIGS. 5C and 5D). These results indicate that imatinib inhibited the proliferation and recruitment of nuocytes but not T cells to the lungs during influenza infection and after the administration of rIL-33.

### Example 3

### Imatinib Inhibits Nuocyte Proliferation and Cytokine Productions in vitro

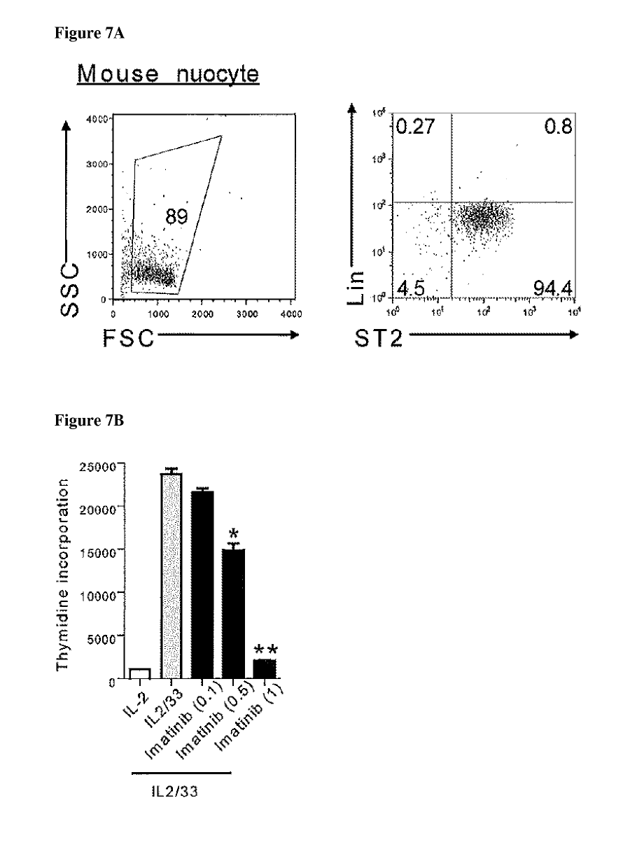

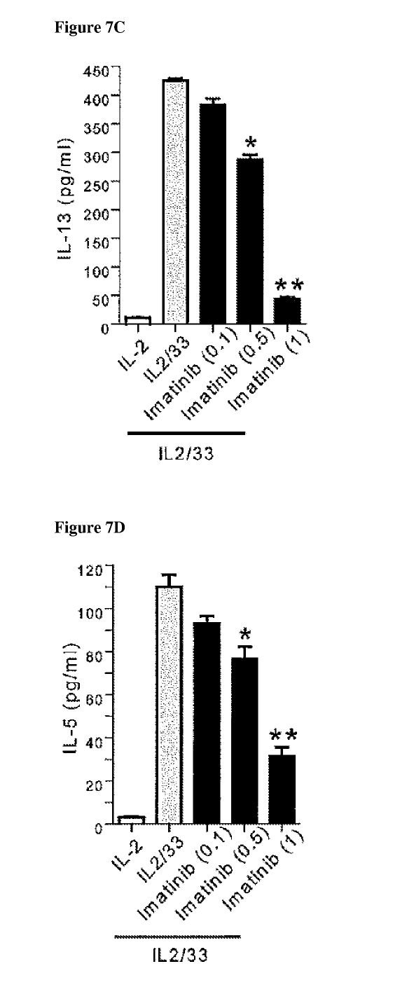

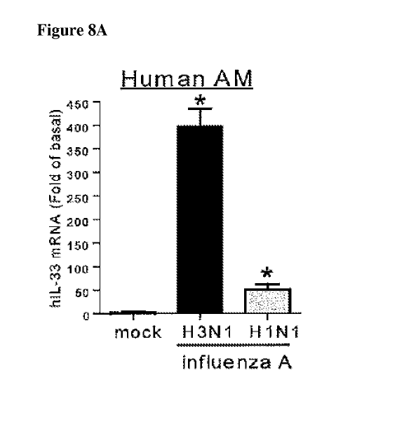

To determine whether imatinib directly affected nuocyte function, nuocytes (Lin⁻ST2⁺ cells) expanded in Rag2^{-/-} mice by in vivo treatment with rIL-33 (1 μg, i.n.) were isolated (gating shown in FIG. 7A). The nuocytes were then cultured in vitro with IL-2 (50 ng/ml), or IL-2 plus rIL-33 (100 ng/ml) for 24hrs in the presence or absence of imatinib (0.1-1 μM). FIG. 7B shows that imatinib blocked the proliferation of mouse lung nuocytes (as shown by a reduction in ³II-thymidine incorporation) in a dose-dependent manner. Furthermore, secretion of IL-13 and IL-5 by nuocytes was reduced by imatinib (FIGS. 7C and 7D), indicating that in vivo treatment with imatinib directly inhibits the proliferation, expansion and cytokine production of lung nuocytes during influenza infection.

### Example 4

### Imatinib Inhibits Human Nuocyte IL-13 Secretion in Vitro

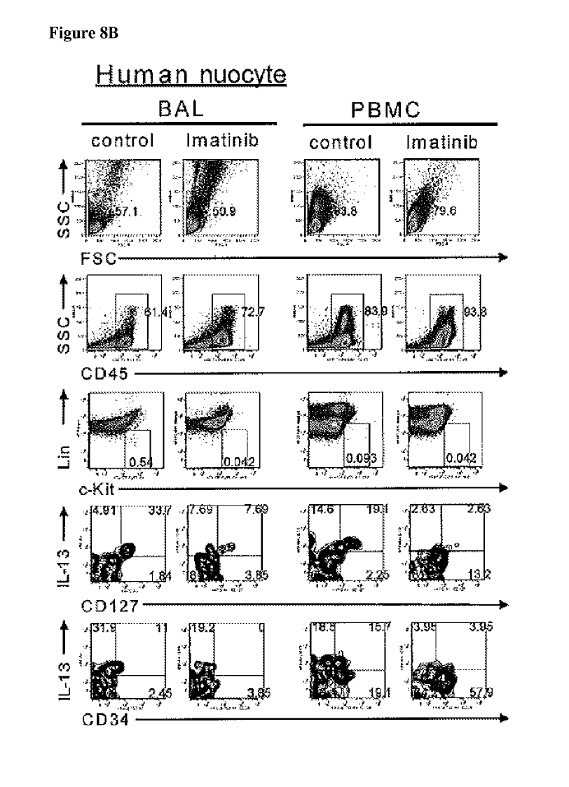

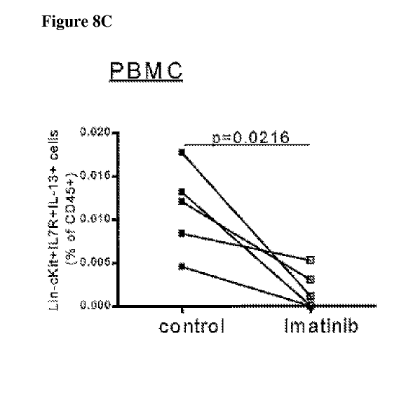

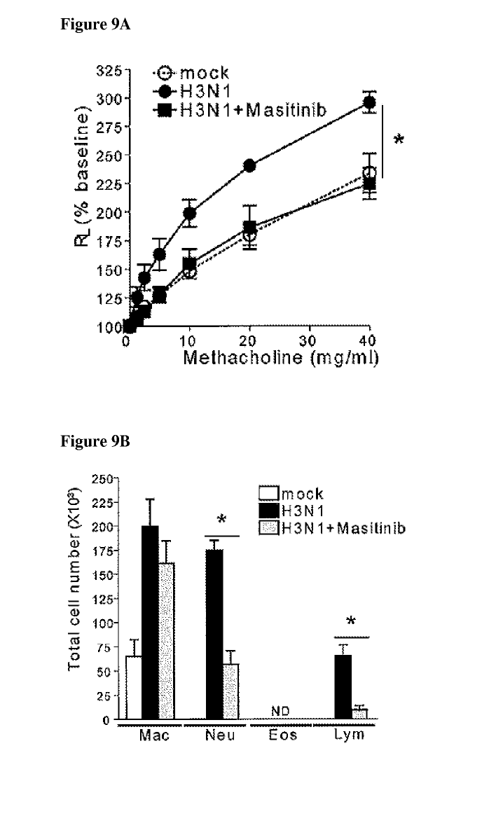

It was next determined whether imatinib is also effective in humans infected with influenza A virus. First it was shown that infection of human alveolar macrophages (AM) with influenza A induced production of IL-33, as it does in mice, which then drives the activation and expansion of nuocytes. Thus, human AMs, isolated from BAL fluid of patients with asthma, expressed significant increases in IL-33 mRNA when infected with influenza A strain H3N1 (M.O.I═5) or H1N1 (M.O.I═1) for 24 hr (FIG. 8A). Furthermore, it was shown that imatinib (1 μM) blocked the production of IL-13 by human nuocytes isolated from the BAL fluid of patients with asthma (FIG. 4B, left panels) stimulated with human rIL-2 (50 ng/ml) plus rIL-33 (100 ng/ml) for 24 hrs. Imatinib (1 μM) also blocked the expansion of IL-13 producing nuocytes in cultures of peripheral blood mononuclear cells (PBMC) from healthy donors (with rIL-2+rIL-33), as seen by a lower percentage of CD45⁺Lin⁻cKit⁺CD127⁺IL-13 producing nuocytes present in the cultures (FIG. 8C, p═0.0216). Since in vivo serum trough imatinib concentrations of 2-4 μM of imatinib are routinely achieved in the blood of patients with chronic myelogenous leukemia (von Mehren, M. et al., Cancer Treat Rev. 37:291-299 (2011)), these results indicate that imatinib therapy can reduce the function of human as well as murine nuocytes.

### Example 5

### Masitinib or Anti-c-Kit mAb Abolishes H3N1-Induced AHR and Inflammation

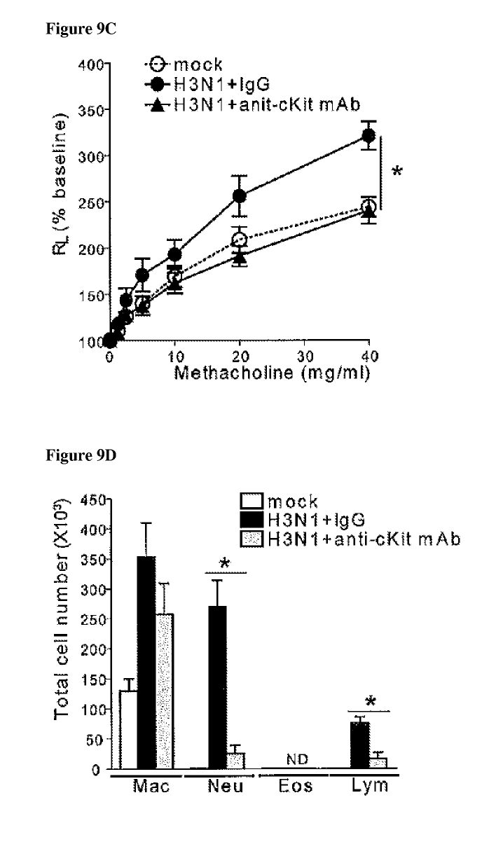

Because imatinib affects several other kinases, it was next examined whether blockade specifically of c-Kit kinase was required to block influenza-induced AHR. FIG. 9 shows that treatment with mastinib (500 μg/mouse on day 1), a c-Kit kinase inhibitor with greater specificity for c-Kit than imatinib, abolished H3N1-induced AIIR and inflammation (FIGS. 9C and 9D). These results indicate that blockade of c-Kit signaling can indeed prevent H3N1-induced AHR and inflammation.

### Example 6

### Imatinib Does Not Affect Viral Clearance or Suppress Bone Marrow Function

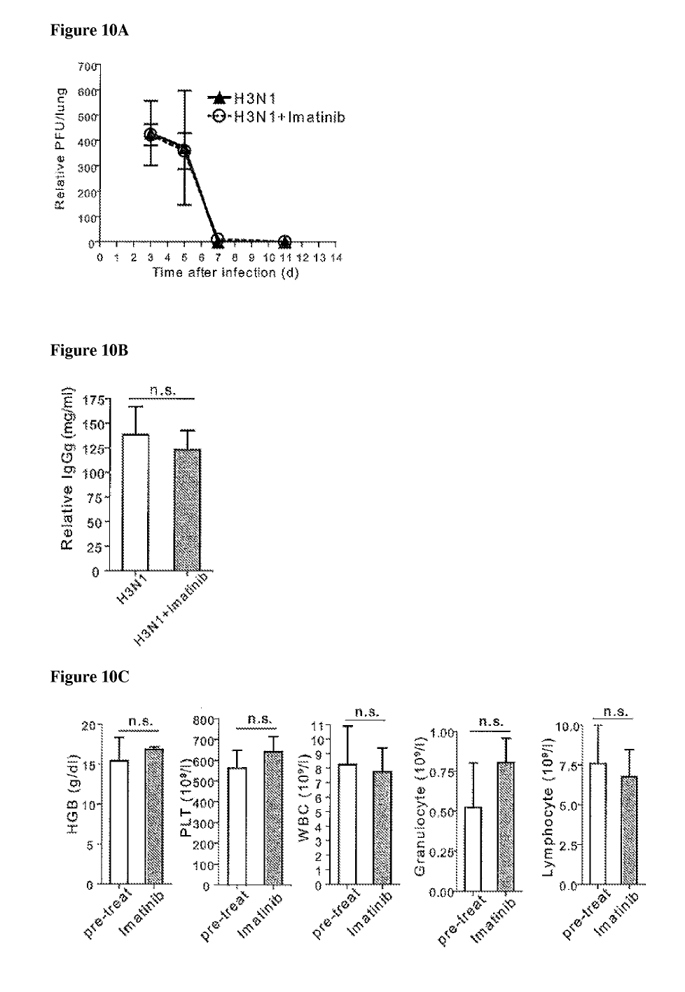

As a potential therapy for acute asthma, it is important to determine if imatinib treatment affects the clearance of influenza A virus or reduces the peripheral white blood cell, red blood cell or platelet count (bone marrow stem cells express c-Kit). Examination of the viral loads in lungs and anti-viral antibody titers showed that imatinib treated mice cleared influenza virus as rapidly as did untreated mice, and that there was no significant reduction in anti-influenza IgG production measured on day 11 (FIGS. 10A and 10B). In addition, treatment with imatinib had no effect on peripheral blood hemoglobin levels, platelet, or on white blood cell, granulocyte or lymphocyte counts (FIG. 10C). These results indicate that short-term treatment with imatinib had no major effects on the anti-viral responses or on gross bone marrow function.

### Example 7

### Imatinib Treatment Abolishes RSV-Induced AHR

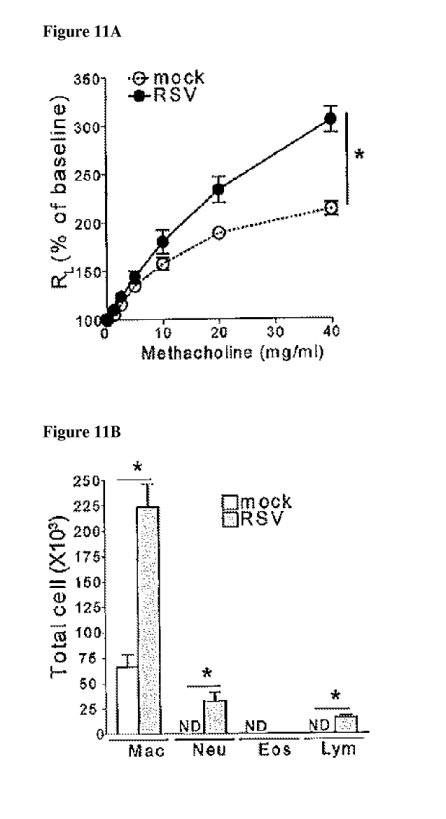

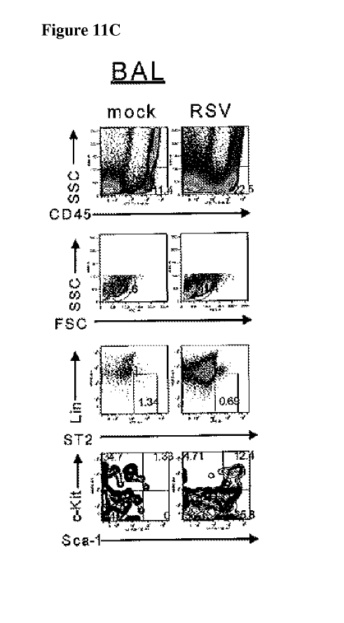

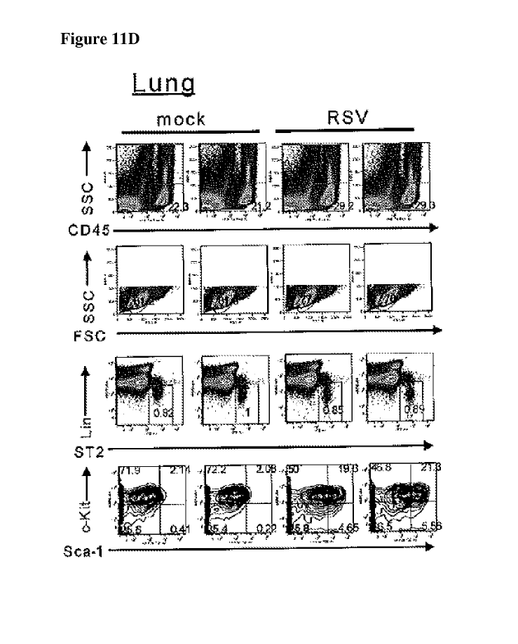

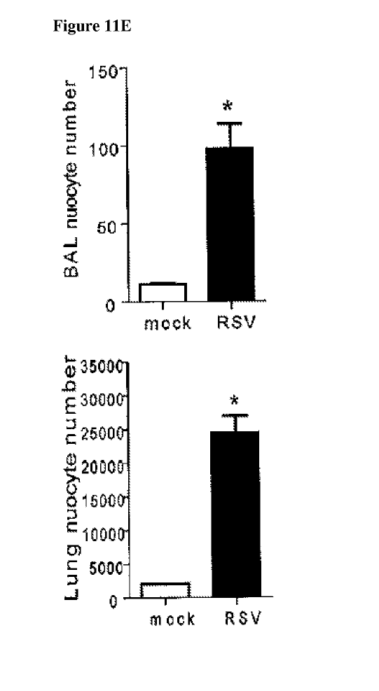

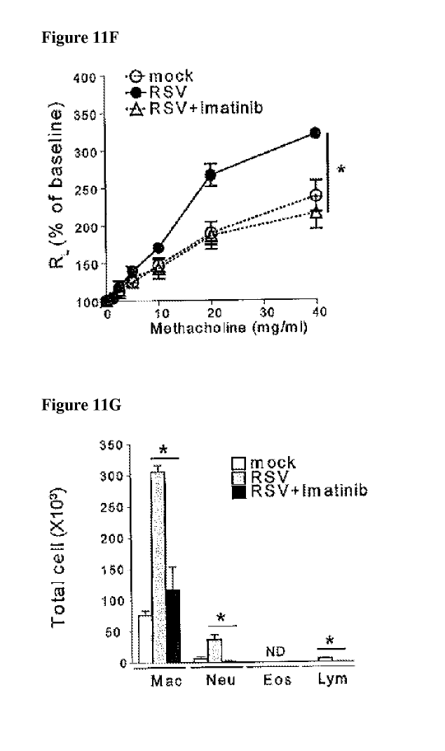

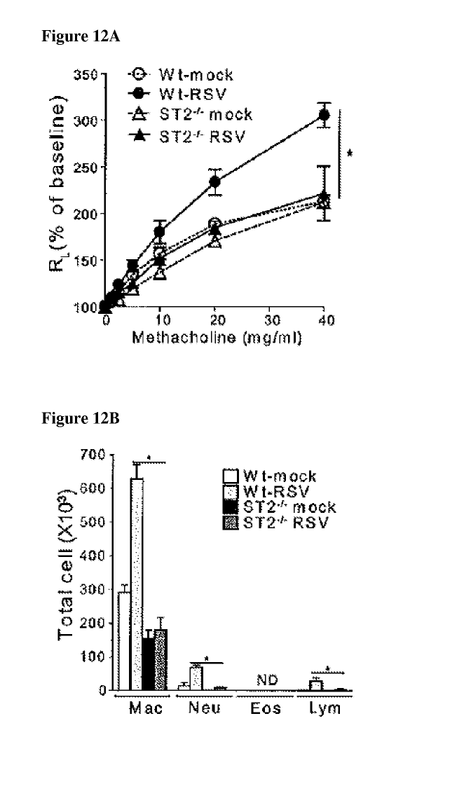

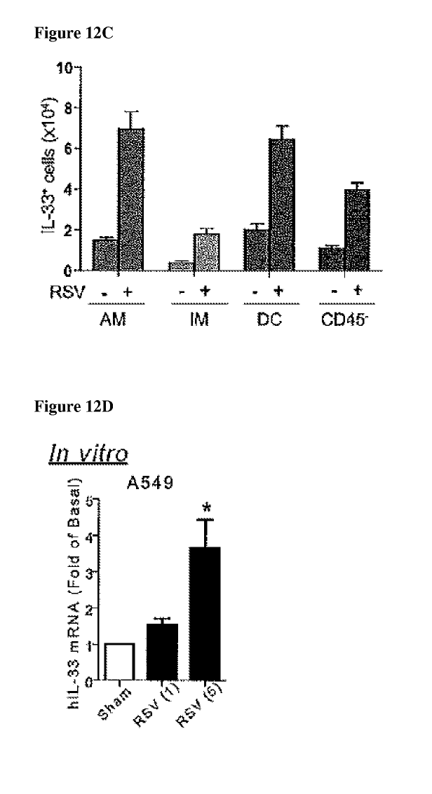

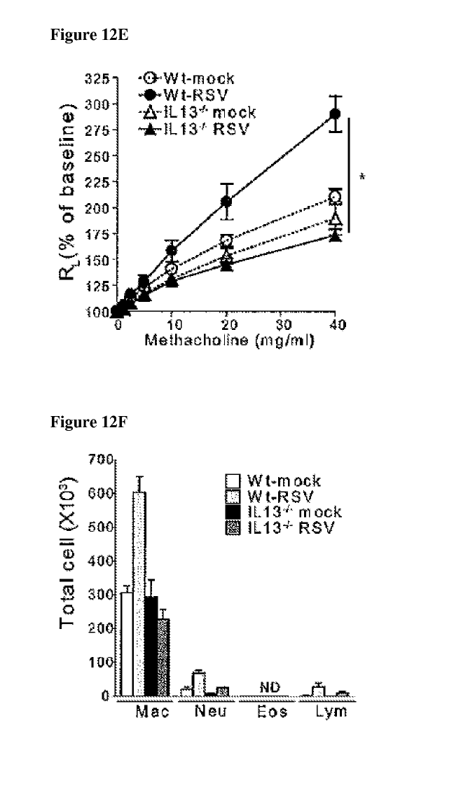

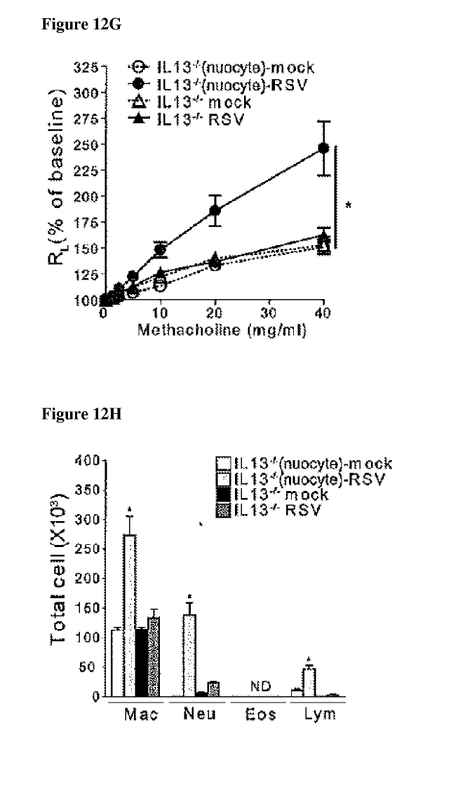

It was next determined whether imatinib was effective in limiting airways disease caused by another respiratory virus, e.g., respiratory syncytial virus (RSV). RSV infection, like influenza, can cause acute asthma symptoms, although the precise mechanisms of RSV induced wheezing is not fully defined. Nevertheless, in a mouse model that replicates many of the features seen in humans with RSV infection, it was shown that RSV infection acutely (within 6 days) induced severe AHR associated with airway neutrophilia (FIGS. 11A and 11B), and with an increase in the number of nuocytes in the airways (FIGS. 11C-11E). Surprisingly, treatment with imatinib on day 1 post infection blocked the development of AHR and inflammation (FIGS. 11F and 11G). An IL-33-ST2-nuocyte axis was required for RSV-induced AIIR, since it did not occur in ST2^{-/-} mice (FIGS. 12A and 12B). Moreover, RSV induced the production of IL-33 in alveolar macrophages, airway DCs, and airway epithelial cells (FIGS. 12C and 12D), which presumably drove the expansion of lung nuocytes. IL-13 producing nuocytes were required for the development of AHR, since RSV infection failed to induce AHR and inflammation in IL-13^{-/-} mice (FIGS. 12E and 12F), and adoptive transfer of IL-13 competent nuocytes into IL-13^{-/-} mice fully restored RSV-induced AHR (FIGS. 12G and 12H). These results evidence that imatinib treatment can block RSV-induced AIIR and further support an important role for nuocytes in viral-induced AIIR.

As demonstrated in the above examples, therapies that target nuocytes can effectively prevent the development of AHR and airway inflammation. It was shown that treatment with c-Kit kinase inhibitors or with anti-c-Kit mAb, blocked the activation of nuocytes, which are required for the development of influenza-induced AHR, and successfully prevented the development of influenza-induced respiratory pathology. Moreover, the inhibition of AHR was equally effective whether therapy was started before or after the initiation of the infection, presumably because the c-Kit kinase inhibitors affected effector cells (nuocytes) rather than infected cells. Since viral infection commonly causes significant respiratory pathology and is a frequent cause of hospitalization in patients with asthma, the results described herein indicate that c-Kit kinase inhibitors will be effective as treatment for patients with virus-associated asthma.

Currently, the mainstay of therapy for asthma relies heavily on inhaled and systemic corticosteroids, which are very effective for allergic asthma, mediated by Th2 cells and eosinophils, but corticosteroids are much less effective for asthma associated with viral infections including influenza. In contrast to allergic asthma, virus-associated asthma involves innate immunity, e.g., nuocytes, alveolar macrophages and neutrophils, which appear to be relatively resistant to corticosteroid treatment. Therefore, anti-c-Kit kinase inhibitors that target nuocytes may preferentially benefit patients with virus-associated asthma but not with allergic asthma, since nuocytes appear to be associated with viral infection and not allergic asthma (Chang, Y. J. et al., Nature Immunol. 12:631-638 (2011)). However, it is likely that a combination of both corticosteroids and c-Kit kinase inhibitors may be more effective than each alone, particularly when asthma exacerbations develop following exposure to both allergen and influenza.

As shown above, imatinib blocking of virus-induced AHR was not due to blockade of other kinases affected by imatinib, such as c-Abl. Treatment with masitinib, a c-Kit kinase inhibitor with greater specificity for c-Kit than imatinib had effects on virus-induced AHR were identical to that of imatinib. Moreover, treatment with anti-c-Kit mAb, which is specific for the c-Kit receptor, also had identical effects as imatinib on virus-induced AHR. These results therefore evidence that the effects of imatinib and masitinib were due to their effects on c-Kit and not on other kinases.

c-Kit kinase is expressed not only by nuocytes, but also by mast cells, eosinophils, some dendritic cells (DCs), and some natural killer (NK) cells (Ray, P. et al., Ann. NY Acad. Sci. 1183:104-122 (2010)). Therefore it is possible that the effects of imatinib or masitinib could be due to their effects on multiple cell types. However, eosinophils are not required in the model of virus-induced AIIR used in the above examples, making it unlikely that imatinib mediated its effects on virus-induced AHR by affecting this non-nuocyte cell type. Furthermore, the above results show that imatinib prevented an increase in the number of nuocytes following viral infection and reduced IL-13 and IL-5 production in mouse and human nuocytes, demonstrating the profound effect of imatinib on nuocytes. Nevertheless, it is possible that imatinib inhibition of mast cells and other cell types contributes to its inhibitory effects on influenza-induced AHR.

The presence of nuocytes in the lungs of humans was suggested by a previous study showing that IL-13 and IL-5 producing non-T non-B cells were present in the sputum of patients with asthma (Allakhverdi, Z. et al., J. Allergy Clin. Immunol. 123:472-478 (2009)), by their presence in human nasal polyp tissue (Mjosberg, J. M. et al., Drug Saf 32:1001-1015 (2009)), and by their presence in healthy human lungs (Monticelli, L.A. et al., Nat. Immunol. 12:1045-1054 (2011)). Viral infection of human alveolar macrophages in vitro induces the production of IL-33 (see supra and Chang, Y. J. et al., Nature Immunol. 12:631-638 (2011)), which has been shown to activate human nuocytes (Moro, K. et al., Nature 463:540-544 (2010); Neill, D. R. et al., Nature 464:1367-1370 (2010); and Saenz, S. A. et al., Nature 464:1371-1376 (2010)). As described herein, imatinib effectively blocks nuocyte activation, e.g., imatinib blocks IL-13 production in human nuocytes activated with IL-33. The potent inhibitory effects of c-Kit kinase inhibitors as observed in the above model of virus-induced AHR indicate that imatinib will be effective in the treatment of patients with asthma triggered by viral infection.

RSV, like influenza, is a leading cause of serious respiratory illness and wheezing in young children and adults (Hall, C. B. et al., N. Engl. J. Med. 360:588-598 (2009); Westerly, B. D. et al., Immunol. Allergy Clin. North Am. 30:523-539, vi-vii (2010); and Falsey, A. R. et al., N. Engl. J. Med. 352:1749-1759 (2005)). Although prophylactic therapy for RSV infection is available for use in high-risk premature infants (anti-RSV mAb), an effective vaccine has not yet been developed for RSV, and hospitalization rates for RSV in children are three times that for influenza (Hall, C. B. et al., N. Engl. J. Med. 360:588-598 (2009)). Surprisingly, treatment of mice with imatinib blocked the development of RSV-induced AHR and inflammation. Like with influenza, RSV-induced AHR required an IL-33-ST2 axis, as well as IL-13 producing nuocytes, although RSV but not influenza infection greatly augmented IL-33 production in airway epithelial cells. Previously, an important role for ST2 in RSV infection was demonstrated, although in that model, Th2 cells were thought to primarily express ST2 and respond to the ST2 ligand (Walzl, G. et al., J. Exp. Med. 193:785-792 (2001)). Nevertheless, the results described herein demonstrate an important role for nuocytes in the pathogenesis viral respiratory disease, and indicate a therapeutic role for c-Kit kinase inhibitors.

Finally, use of c-Kit kinase inhibitors (e.g., imatinib, masitinib, c-Kit antibodies, and the like) as therapy for virus-induced asthma is made more beneficial by the fact that prolonged use of these agents is associated with relatively few side effects (Wolf, D. Et al., Drug Saf 32:1001-1015(2009); Breccia, M. et al., Curr. Cancer Drug Targets (2012); and Breccia, M. et al., Crit. Rev. Oncol. Hematol. (2012)). Treatment for acute viral infection might involve only a few doses of imatinib or masitinib, making side effects even less likely. In mice, short term treatment with imatinib did not reduce the peripheral blood neutrophils, lymphocytes, monocytes and RBC, cell types that are all derived from c-Kit expressing bone marrow stem cells, while it greatly reduced the number of nuocytes in the lungs of infected mice. Moreover, unlike corticosteroids, c-Kit kinase inhibitors target nuocytes, rather than T cells and B cells (or macrophages), and should not limit the development of anti-viral immunity adaptive immunity. Importantly, c-Kit kinase inhibitors do not appear to affect influenza-induced IL-33 production, and should not affect T cell function, in contrast to anti-IL-33 mAb or anti-ST2 mAb therapies, which might reduce anti-viral T cell responses, since IL-33 enhances anti-viral CD8 effectorcell development (Bonilla, W. V. et al., Science 335:984-989 (2012)). Nuocytes have been suggested to enhance the repair of viral induced airway injury by producing amphiregulain (Monticelli, L. A. et al., Nat. Immunol. 12:1045-1054 (2011)). Not wishing to be bound by any theory, it is possible that nuocyte-targeted therapy that prevents airway inflammation and preserves anti-viral adaptive immunity may lessen the need for the nuocyte post-infection repair function.

The findings described herein establish that c-Kit kinase inhibitors targeting nuocytes are extremely effective in treating and preventing virus-associated asthma. Viral infection (e.g., influenza and RSV infection) still causes significant morbidity and mortality in asthma patients, and there is a significant unmet need for an effective therapy for treating virus-associated asthma. The above results clearly demonstrate that use of c-Kit kinase inhibitors provides a solution to this unmet need. Therapy with c-Kit kinase inhibitors is therefore a significant advance in the treatment of patients suffering from virus-induced respiratory disease (e.g., virus-induced asthma).

The results reported herein were obtained using the following methods and materials.

### Animals and Reagents

Wild-type BALB/c ByJ and Rag2^{-/-} mice on the BALB/c background were purchased from The Jackson Laboratory. IL-13−/− and ST2−/− mice were provided by Andrew McKenzie, Cambridge, UK (Townsend, M. J. et al., J. Exp. Med. 191:1069-1076 (2000)). The Animal Care and Use Committee at Children's Hospital Boston approved all animal protocols. Imatinib and masitinib were obtained from LC Laboratories (Woburn, Mass.).

Influenza Virus Infection in vivo

6 to 8-week-old adult female mice were anesthetized with 3% isoflurane and inoculated intranasally (i.n.) with influenza A virus (strain Mem/71 (H3N1)) in 50 μl PBS for adult mice. The virus was a reassortant influenza virus strain carrying the hemagglutinin of A/Memphis/1/71 (II3) and the neuraminidase of A/Bellamy/42 (N1). The virus was grown and harvested from 10-d embryonated chicken eggs as described (Baumgarth, N. et al., J. Virol. 68:7575-7581 (1994)). The dose of virus used (1.2×10⁴ PFU/mouse) causes nonlethal pneumonia of adult mice with complete virus clearance around day 7 after infection. Control (mock infected) mice were treated with i.n. allantoic fluid (A.F) diluted 1:500 in PBS. Viral titers in the lung were assessed by removing the lungs at specified times after initiation of infection. The lungs were homogenized in 1 ml PBS and frozen until processed and analyzed by qRT-PCR. To assess influenza virus loads in lungs of infected mice, total RNA was extracted from aliquots of frozen lung homogenates at indicated times after influenza virus infection using standard RNA isolation kits (Qiagen, Carlsbad, Calif.). Viral positive-stranded mRNA for nuclear protein, i.e., indicating the presence of replicating virus, was amplified and quantified via single-round qRT-PCR using an ABI 7900 real-time PCR system. RNA from virus-containing allantoic fluid of embryonated hen eggs, containing known plaque-forming units (PFU) of influenza virus, determined by Marbin-Darby Canine Kidney plaque assay, was co-purified and served as standard. Relative PFU of lung samples were determined by comparisons to a standard curve established with amplification of a dilutions series of the positive control.

### Influenza Virus Infection of Human Cells in Vitro

Human alveolar macrophages were isolated from the bronchoalveolar lavage fluid of adult asthmatic donors. Alveolar macrophages (AM) (5×10⁵ cells/well in 24 well plates) were stimulated with live influenza virus (Mem71; H3N1) in 0.1% BSA/PBS. 1 hr after stimulation, cells were washed thoroughly and incubated in complete media for 24 hr, after which the cells were harvested for analysis. The human subjects institutional review boards at the relevant institution approved all applicable protocols.

### In Vitro Culture of Human Nuocytes

Human peripheral blood mononuclear cells (PBMCs) from normal volunteers or nuocytes in BAL fluid from patients with asthma were isolated and cultured with IL-2 (50 ng/ml) and IL-33 (100 ng/ml) for 24 hrs, in the presence or absence of imatinib (1 μM). Culture supernatants and cells were assessed for production of IL-33 and IL-13, respectively.

### Measurement of Airway Responsiveness (AHR) in the Influenza Virus Model

5 days after H3N1 virus or mock infection, mice were anesthetized with 50 mg/kg pentobarbital and instrumented for the measurement of pulmonary mechanics (BUXCO Electronics, Wilmington, N.C.). Mice were tracheostomized, intubated, and mechanically ventilated at a tidal volume of 0.2 ml and a frequency of 150 breath/min, as previously described (Akbari, O. et al., Nature Medicine 9:582-588 (2003)). Baseline lung resistance (RL) and responses to aerosolized saline (0.9% NaCl) were measured first, followed by responses to increasing doses (0.125 to 40 mg/ml) of aerosolized acetyl-β-methylcholine chloride methacholine (Sigma-Aldrich, St. Louis, Mo.). The three highest values of RL obtained after each dose of methacholine were averaged to obtain the final values for each dose.

### RSV-Induced AHR Model

Human RSV strain A2 (American Type Culture Collection) was propagated in Hep-2 cells maintained in Eagle's MEM containing 5% heat-inactivated FBS. The infected cells were harvested after 3-4 days, cells were lysed sonically, and the virus was separated from cellular debris under endotoxin-free conditions by centrifugation as described previously (Bera, M. M. et all., J. Immunol. 187:4245-4255 (2011)). Titers were determined by standard plaque assay on Hep-2 cells (Prince, G. A. et al., Am. J. Pathol. 93:771-791 (1978)). Mice were inoculated under light anesthesia (isofluorane) by intranasal instillation of 10⁶ PFU of purified virus in 75 μl endotoxin-free PBS. Sham-infected animals were inoculated with lysed HEp2 cells under identical conditions.

### OVA-Induced AHR Model

To induce AHR, BALB/c mice were sensitized with 100 μg of OVA (Sigma-Aldrich, St. Louis, Mo.) in alum administered i.p (d. 0). After sensitization, mice were exposed to intranasal antigen (50 μg OVA/d) or normal saline for 1 day (d. 7; single dose challenge protocol), or for 3 consecutive days (days 7, 8, 9). AHR was assessed on the day after last OVA-challenge. Control mice received i.p. injection of PBS and intranasal administrations of normal saline.

### RSV Induced AHR

Human RSV strain A2 (American Type Culture Collection) was propagated in Hep-2 cells maintained in Eagle's MEM containing 5% heat-inactivated FBS. The infected cells were harvested after 3-4 days, lysed sonically, and the virus was separated from cellular debris under endotoxin-free conditions by centrifugation as described previously (Bera, 2011 #2665). Titers were determined by standard plaque assay on Hep-2 cells (Prince, 1978 #2666). Mice were inoculated under light anesthesia (isofluorane) by intranasal instillation of 10⁶ PFU of purified virus in 75 μl endotoxin-free PBS. Sham-infected animals were inoculated with lysed HEp2 cells under identical conditions.

### Collection and Analysis of Bronchoalveolar Lavage (BAL) Fluid

Immediately after the AHR measurement, mice were euthanized and the lungs were lavaged twice with 0.5 ml of PBS, and the fluid was pooled. Cells in BAL fluid were counted and analyzed, as previously described (Hansen, G. et al., J. Clin. Invest. 103:175-183 (1999)). The relative number of different types of leukocytes was determined from slide preparations of BAL fluid stained with Diff-Quik solution (Dade Behring, Tarrytown, N.Y.).

### Lung Cell Isolation

Whole lungs were flushed with PBS injected into the right ventricle, removed, and rinsed in PBS. The lungs were then diced on a wax board before incubating in 9.6 ml of RPMI 1640 medium with 0.1% DNase I (fraction IX; Sigma-Aldrich) and 1.6 mg/ml collagenase (CSL4; Worthington Biochemicals, Lakewood, N.J.) at 37° C. on an orbital shaker for 30 min. The digest was passed multiple times through an 18-gauge needle and allowed to incubate for another 30 min before filtered. RBCs were removed by 4-min incubation in lysis buffer (Sigma-Aldrich, St. Louis, Mo.) at room temperature. Single-cell suspensions of spleen lymphocytes were obtained by mechanical disruption and RBC lysis.

### Histopathologic Analysis

The lungs were taken from mice, infused with 10% formalin and embedded in paraffin. Lung sections were cut (5 μm thick) and stained with hematoxylin/eosin (HE) for optic microscopy examination

### Flow Cytometry

Cells were preincubated with anti-Fcy blocking mAb (2.4G2) and washed before staining. Cells were stained with anti-mouse PE-Texas red-conjugated CD45, FITC-conjugated CD3, CD19, CD11b, CD11c, CD49b, F4/80, Fc□R1; APC conjugated cKit,; PerCP conjugated cKit, Alexa Fluor 700-conjugated Sca1; PE conjugated biotinylated anti-ST2 antibodies. CD1d tetramers loaded with PBS57 (□-GalCer analog) (unloaded CD1d tetramers as control in all experiments) from the NIH tetramer facility, Emory University (Atlanta, Ga.) were also used. For intracellular staining, after permeabilization (Cytofix/Cytoperm kit; BD Biosciences, Franklin Lakes, N.J.), cells were incubated with A647-conjugated IL-13 or the respective isotype control antibodies (eBioscience, San Diego, Calif.). Cells were analyzed on a BDCanto flow cytometer (BD Biosciences, Franklin Lakes, N.J.) using FlowJo 8.3.3 software (Tree Star, Inc., Ashland, Oreg.).

### Statistical Tests

Differences between groups with parametric distributions were analyzed by Student's 2 tailed t test; otherwise, the Mann-Whitney U test was used. Data represent mean±SEM. Significance for all statistical tests was shown in figures for P≦0.05 (*), P≦0.01 (**) and P≦0.001 (***).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

### INCORPORATION BY REFERENCE

All patents and publications mentioned in this specification are herein incorporated by reference to the same extent as if each independent patent and publication was specifically and individually indicated to be incorporated by reference.

1. A method for preventing or treating respiratory pathology, airway inflammation, airway hyperreactivity (AHR) or acute asthma in a subject, wherein the method comprises administering an effective amount of a c-Kit kinase inhibitor to the subject.

2-4. (canceled)

5. The method of claim 1, wherein the subject has, is susceptible to, or is at risk of developing a viral infection.

6. The method of claim 5, wherein administration of the c-Kit kinase inhibitor prevents or treats virus-induced respiratory pathology, virus-induced airway inflammation, or virus-induced AHR in the subject.

7. A method selected from the group consisting of:
a method for inhibiting nuocyte activation or lymphokine production in a subject, wherein the method comprises administering to a subject having or at risk of developing a viral infection a c-Kit kinase inhibitor, thereby inhibiting nuocyte activation or lymphokine production in the subject;
a method for inhibiting lymphokine production, wherein the method comprises contacting a cell with a c-Kit kinase inhibitor, thereby inhibiting lymphokine production by the cell;
a method for inhibiting nuocyte activation, wherein the method comprises contacting a nuocyte with a c-Kit kinase inhibitor, thereby inhibiting nuocyte activation;
a method for preventing or treating influenza-induced airway hyperreactivity (AHR) or airway inflammation in a subject, wherein the method comprises administering an effective amount of a c-Kit kinase inhibitor to the subject;
a method for preventing or treating respiratory syncytial virus (RSV)-induced airway hyperreactivity (AHR) or airway inflammation in a subject, wherein the method comprises administering an effective amount of a c-Kit kinase inhibitor to the subject;
a method for treating acute influenza infection in a subject, wherein the method comprises administering an effective amount of a c-Kit kinase inhibitor to the subject to reduce airway hyperreactivity (AHR) or airway inflammation in the subject;
a method for treating acute respiratory syncytial virus (RSV) infection in a subject, wherein the method comprises administering an effective amount of a c-Kit kinase inhibitor to the subject to reduce airway hyperreactivity (AHR) or airway inflammation in the subject; and
a method for treating acute asthma in a subject, wherein the method comprises administering an effective amount of a c-Kit kinase inhibitor to the subject to reduce airway hyperreactivity (AHR) or airway inflammation in the subject.

8. (canceled)

9. The method of claim 7, wherein the lymphokine is IL-13 or IL-5.

10. The method of claim 5, wherein the viral infection is of a virus that causes asthma symptoms.

11. The method of claim 10, wherein the virus is respiratory syncytial virus (RSV), influenza, rhinovirus, parainfluenza, adenovirus, coronavirus, metapneumovirus, or bocavirus.

12. The method of claim 1, wherein the subject is a mammal.

13. The method of claim 1, wherein the subject is a human.

14. (canceled)

15. The method of claim 1, wherein the subject is selected from the group consisting of a pregnant female, a young subject or an infant subject, a subject less than 10 years of age, an elderly subject, a subject at least 65 years old and a subject having an underlying medical condition.

16-20. (canceled)

21. The method of claim 15, wherein the underlying medical condition is a neurological condition, a heart condition, or a respiratory condition.

22. The method of claim 15, wherein the underlying condition is asthma.

23. The method of claim 5, wherein the viral infection is an acute viral infection.

24. The method of claim 1, wherein the c-Kit kinase inhibitor is administered to the subject for 1-3, 1-5, or 1-7 days.

25. (canceled)

26. The method of claim 1, wherein the subject does not respond to corticosteroid therapy or the method reduces wheezing, shortness of breath, chest tightness, and coughing in the subject.

27-29. (canceled)

30. The method of claim 7, wherein;
the cell is a nuocyte;
the c-Kit kinase inhibitor is contacted with the nuocyte for 1-3, 1-5, or 1-7 days;
the c-Kit kinase inhibitor is a compound of Formula I, a compound of Formula II, dasatinib; imatinib; sunitinib; axitinib; pazopanib; cabozantinib; dovitinib; telatinib; Ki8751; OSI-930; AMN107; midostaurin; amuvatinib; tivozanib; regorafenib; vatalanib; masitinib; motesanib; or a salt, analog, or derivative thereof;
the c-Kit kinase inhibitor is imatinib; masitinib; or a salt, analog, or derivative thereof;
the c-Kit kinase inhibitor inhibits c-Kit kinase activity;
the c-Kit kinase inhibitor is an antibody or antibody fragment;
the c-Kit kinase inhibitor is an antibody or antibody fragment that selectively binds c-Kit;
the c-Kit kinase inhibitor is a polyclonal antibody;
the c-Kit kinase inhibitor is a monoclonal antibody;
the c-Kit kinase inhibitor is a humanized antibody or antibody fragment;
the c-Kit kinase inhibitor is an inhibitory nucleic acid molecule;
the c-Kit kinase inhibitor is an siRNA, shRNA or antisense nucleic acid molecule that reduces expression of c-Kit;
the method further comprises administering at least one additional anti-asthma medication;
the method further comprises administering at least one additional anti-asthma medication selected from the group consisting essentially of a corticosteroid, a beta-agonist, a leukotriene modifier, a mast cell stabilizer, theophylline, an immunomodulator, an anti-IgE therapy, and an anti-cholinergic;
the method further comprises administering a corticosteroid;
the acute asthma is caused by influenza or respiratory syncytial virus (RSV) infection;
the c-Kit kinase inhibitor is an antibody or antibody fragment that selectively binds c-Kit, wherein binding of the antibody or antibody fragment to c-Kit inhibits c-Kit kinase activity; or
the method reduces wheezing, shortness of breath, chest tightness, and coughing in the subject.

31-55. (canceled)

56. A pharmaceutical composition comprising a c-Kit kinase inhibitor for use in a method of claim 1.

57. The pharmaceutical composition of claim 56, wherein the pharmaceutical composition further comprises a pharmaceutically acceptable carrier, diluent, or excipient.

58. A kit selected from the group consisting of:
a kit for preventing or treating virus-induced respiratory pathology, the kit comprising a c-Kit kinase inhibitor;
a kit for preventing or treating virus-induced airway inflammation, the kit comprising a c-Kit kinase inhibitor;
a kit for preventing or treating virus-induced airway hyperreactivity (AHR), the kit comprising a c-Kit kinase inhibitor;
a kit for preventing or treating acute asthma, the kit comprising a c-Kit kinase inhibitor;
a kit for inhibiting nuocyte activation, the kit comprising a c-Kit kinase inhibitor; and
a kit for inhibiting lymphokine production, the kit comprising a c-Kit kinase inhibitor.

59-63. (canceled)

64. The kit of claim 58, wherein:
the kit further comprises directions for using the c-Kit kinase inhibitor in the method of claim 1;
the c-Kit kinase inhibitor is dasatinib; imatinib; sunitinib; axitinib; pazopanib; cabozantinib; dovitinib; telatinib; Ki8751; OSI-930; AMN107; midostaurin; amuvatinib; tivozanib; regorafenib; vatalanib; masitinib; motesanib; or a salt, analog, or derivative thereof;
the c-Kit kinase inhibitor is imatinib; masitinib; or a salt, analog, or derivative thereof;
the c-Kit kinase inhibitor is an antibody or antibody fragment that inhibits c-Kit kinase activity;
the c-Kit kinase inhibitor is an the antibody or antibody fragment that selectively binds c-Kit;
the c-Kit kinase inhibitor is a polyclonal antibody;
the c-Kit kinase inhibitor is a monoclonal antibody;
the c-Kit kinase inhibitor is a humanized antibody or antibody fragment;
the c-Kit kinase inhibitor is an inhibitory nucleic acid molecule;
the c-Kit kinase inhibitor is an siRNA, shRNA or antisense nucleic acid molecule that reduces expression of c-Kit;
the kit further comprises at least one additional anti-asthma medication;
the kit further comprises at least one additional anti-asthma medication selected from the group consisting essentially of a corticosteroid, a beta-agonist, a leukotriene modifier, a mast cell stabilizer, theophylline, an immunomodulator, an anti-IgE therapy, and an anti-cholinergic; or
the kit further comprises a corticosteroid.

65-75. (canceled)
